(19) United States
(12) Patent Application Publication
(10) Pub. No.: US 2019/0241879 A1
(43) Pub. Date: Aug. 8, 2019

(54) METHODS AND COMPOUNDS FOR GENE INSERTION INTO REPEATED CHROMOSOME REGIONS FOR MULTI-LOCUS ASSORTMENT AND DAISYFIELD DRIVES

(71) Applicants: Massachusetts Institute of Technology, Cambridge, MA (US); President and Fellows of Harvard College, Cambridge, MA (US)

(72) Inventors: Kevin ESVELT, Newton, MA (US); Jianghong MIN, Boston, MA (US); Charleston NOBLE, Cambridge, MA (US)

(73) Assignees: Massachusetts Institute of Technology, Cambridge, MA (US); President and Fellows of Harvard College, Cambridge, MA (US)

(21) Appl. No.: 16/331,772

(22) PCT Filed: Sep. 9, 2017
(86) PCT No.: PCT/US17/50857

§ 371 Date: Mar. 8, 2019

Related U.S. Application Data
(60) Provisional application No. 62/385,679, filed on Sep. 9, 2016.
(60) Provisional application No. 62/423,752, filed on Nov. 17, 2016.

(51) Int. Cl. C12N 9/22; C12N 15/10; A01K 67/027; A01K 67/033

(52) U.S. Cl. CPC C12N 9/22; C12N 15/10; A01K 67/0275; A01K 67/0339; A01K 2267/01; C12N 2800/80; A01K 2217/15; A01K 2227/105; A01K 2227/706; C12N 2310/20

(57) ABSTRACT

The invention relates, in part, to methods to design and construct gene drives such as daisy chain gene drives, suppression gene drives, and other types of gene drives that may be included in cell lines and organisms.

### RELATED APPLICATIONS

This application claims benefit under 35 U.S.C. § 119(e) of U.S. Provisional application Ser. No. 62/385,679 filed Sep. 9, 2016, and U.S. Provisional application Ser. No. 62/423,752 filed Nov. 17, 2016 the disclosure of each of which is incorporated by reference herein in its entirety.

### FIELD OF THE INVENTION

The invention relates, in part, to methods of designing and constructing gene drive systems and daisyfield gene drive systems and their inclusion and use in cell lines and organisms.

### BACKGROUND OF THE INVENTION

To date, gene drive elements based on Cas9 have been demonstrated in yeast (DiCarlo, J. E. et al., Nat Biotechnol. 2015 December; 33(12):1250-1255), fruit flies (Gantz, V. & Bier, E. 2015 Science 24 April: Vol. 348, Issue 6233, pp. 442-444), and two species of mosquitoes (Gantz, V. et al., 2015 PNAS Vol. 112 no. 49 E6736-E6743, doi: 10.1073/pnas.1521077112, Hammond, A. et al., Nat Biotechnol. 2015 Dec. 7; doi:10.1038/nbt.3439). Although functional gene drives have been prepared, few are suitable for efficient and safe inclusion in wild populations of organisms.

### SUMMARY OF THE INVENTION

According to an aspect of the invention, methods of preparing an engineered organism are provided, the methods including: inserting one or more DNA cassettes each comprising an independently preselected DNA sequence into a plurality of repeated regions in the genome of an organism of a strain to prepare a first engineered organism, wherein a means of inserting the preselected DNA sequence comprises at least one of: a) using transposons to pseudo-randomly incorporate a plurality of copies of the preselected DNA sequence into the genome of the organism; and b) using a nuclease-class enzyme to cut one or more strands of a predetermined natural sequence repeat in the genome of the organism and inducing homologous recombination of the preselected DNA sequence with the predetermined natural sequence. In some embodiments, the preselected DNA cassette is a site DNA cassette comprising DNA of one or more small recombinase sites. In some embodiments, the preselected DNA cassette is an insertion DNA cassette, and the method further comprises inserting one or more of the insertion DNA cassettes into the site DNA cassettes harboring the one or more small recombinase sites using one or more of an appropriate recombinase enzyme. In some embodiments, the preselected DNA cassette is an insertion DNA cassette and comprises a DNA sequence encoding a desired organism trait, and the method further comprises: c) preparing a plurality of the engineered organism comprising a plurality of one or more inserted DNA sequences conferring the desired organism trait; d) releasing the plurality of the prepared engineered organisms into the wild, wherein the release introduces the desired trait into a local population of the organism. In some embodiments, the preselected DNA cassette is an insertion DNA cassette, and two or more of the insertion DNA cassettes are inserted, wherein: (i) a first insertion DNA cassette comprises one or more CRISPR components and a plurality of the first insertion DNA cassettes is inserted into the plurality of repeated regions in the genome of the organism; (ii) a second insertion DNA cassette is inserted into a single site in the genome of the organism; wherein the second insertion DNA cassette comprises DNA encoding: (a) one or more cargo genes, and optionally encoding (b) an independently selected CRISPR component that differs from that in the first insertion DNA cassette. In some embodiments, in some embodiments, the CRISPR components comprise a nuclease and the method further comprises the nuclease inducing conversion of one or more germline cells that are heterozygous for the second insertion DNA cassette into homozygotes by nuclease-mediated cutting and repair by homologous recombination, thereby copying the second insertion DNA cassette. In some embodiments, the first insertion cassette comprises a DNA encoding one or more guide RNAs and a plurality of the first insertion cassette is inserted throughout the genome of the organism, and the second insertion cassette comprises a DNA encoding the nuclease gene(s) and one or more cargo genes, and the second insertion cassette is copied in the presence of at least one guide RNA cassette. In some embodiments, the first insertion cassette comprises a DNA encoding the nuclease gene and a plurality of the first insertion cassette is inserted throughout the genome of the organism, and the second insertion cassette comprises one or more guide RNAs and one or more cargo genes, and the second insertion cassette is copied in the presence of at least one copy of the nuclease gene. In some embodiments, the first insertion cassette comprises a DNA encoding one or more guide RNAs and one or more corresponding nuclease enzymes and a plurality of the first insertion cassette is inserted throughout the genome of the organism, and the second insertion cassette comprises a DNA encoding one or more cargo genes, and the second insertion cassette is copied in the presence of at least one copy of the first insertion cassette. In some embodiments, the method also includes generating a transgenic strain of the engineered organism wherein the genome of the organism comprises a plurality of copies of first insertion cassette comprising the CRISPR components and one copy of the second insertion DNA cassette comprising one or more cargo genes. In some embodiments, the method also includes releasing a plurality of organisms of the transgenic strain into the wild, wherein the release efficiently introduces copies of the second insertion DNA cassette into the local population. In some embodiments, the nuclease-class enzyme is a nickase or a nuclease. In some embodiments, a plurality is: 2 or more, 3 or more, 4, or more, 5 or more, or 6 or more. In some aspects, the invention includes preparing and releasing a plurality of the prepared engineered organisms into the wild.

According to an aspect of the invention, methods of generating a threshold-dependent gene drive system by engineered underdominance in a population of organisms are provided, the methods including in one or more organisms in the population, positions of a first haploinsufficient gene on a first chromosome in a cell of the organism with a second haploinsufficient gene in an unlinked locus, such as on a second chromosome in the cell of the organism. In some embodiments, the first and second haploinsufficient genes are ribosomal genes. In some embodiments, neither the first nor the second haploinsufficient genes are ribosomal genes. In some embodiments, only one of the first and the second haploinsufficient genes is a ribosomal gene. In some embodiments, the method also includes preparing for the exchange of the first and second haploinsufficient genes by: (a) selecting a first candidate haploinsufficient gene; (b) inserting into at least one cell a first and a second independently selected recombinase site into the chromosome comprising the first candidate haploinsufficient gene, wherein the inserted first and second independently selected recombinase sites flank the candidate haploinsufficient gene and associated expression signals; (c) selecting a second candidate haploinsufficient gene, wherein the second candidate haploinsufficient gene is positioned in an unlinked locus, such as on a different chromosome, relative to the first candidate haploinsufficient gene; and (d) inserting into the at least one cell a third and a fourth independently selected recombinase site into the chromosome comprising the second candidate haploinsufficient gene, wherein the inserted third and fourth independently selected recombinase sites flank the second candidate haploinsufficient gene and associated expression signals. In some embodiments, the method also includes assessing presence and position of each of the first, second, third, and fourth recombinase sites. In some embodiments, assessing comprises amplification and sequencing methods, and optionally wherein the amplification method comprises a polymerase chain reaction method. In some embodiments, the method also includes contacting each of the first, second, third, and fourth independently selected inserted recombinase sites with a recombinase specific for the first, second, third, and fourth recombinase site, respectively, under suitable conditions for recombination activity at the contacted recombinase sites, such that the two haploinsufficient genes exchange positions. In some embodiments, the method also includes determining the presence and positions of the first and second haploinsufficient genes. In some embodiments, the determining comprises amplification and sequencing methods, and optionally the amplification method comprises a polymerase chain reaction method. In some embodiments, the at least one cell is in an organism, wherein the cell is optionally a germline cell. In some embodiments, the at least one cell is one or more of: a zygote, a gamete, and a cell that can give rise to a gamete. In some embodiments, the method also includes crossing the organism comprising the inserted recombinase sites with a wild-type of the organism; and assessing the outcome of the recombinase activity and putative underdominance in the organism. In some embodiments, the assessing comprises one or more of: quantifying the number of offspring from the cross, the presence and position of one or more of the first and second candidate haploinsufficient genes in the offspring. In some embodiments, the assessing comprises one or more of amplification methods, hybridization methods, and the use of detectable labels or marker genes inserted adjacent to one or more of the haploinsufficient genes. In some embodiments, two of the independently selected recombinase sites are mutually compatible, and optionally, the four independently selected recombinase sites comprise two of one type of recombinase site and two of another type of recombinase site. According to an aspect of the invention, one or more of an organism or population of organisms comprising a threshold-dependent gene drive system is provided. In some embodiments, the organism or a plurality of the organism is released into the wild.

According to an aspect of the invention methods of generating a toxin-antitoxin gene drive system are provided, the methods including (a) inserting into a genome of an organism one or more DNA cassettes encoding a toxin in the form of one or more preselected CRISPR nuclease genes, one or more corresponding guide RNAs, and appropriate expression signals, wherein when expressed, the preselected CRISPR genes cut and disrupt a target gene required for viability or fertility of the organism, and (b) inserting into the genome of the organism one or more DNA cassettes encoding one or more cargo genes and an antitoxin comprising at least one copy of one or more recoded versions of the target gene, wherein the recoded versions of the target gene comprise one or more sequence modifications in the nucleic acid sequence of the target gene wherein the one or more modifications prevent cutting of the recoded gene by the nuclease and do not alter the amino acid sequence of the expressed recoded target gene from that of the expressed target gene, and wherein expressing the one or more recoded target genes is sufficient to rescue viability or fertility. In some embodiments, the toxin-antitoxin system comprises a 2-locus threshold-dependent underdominance gene drive system, wherein a first DNA cassette comprises sequences encoding a toxin A and an antitoxin B and optionally one or more cargo genes, and a second DNA cassette comprises sequences encoding a toxin B and an antitoxin A and optionally one or more cargo genes, and wherein an offspring of an organism comprising the toxin-antitoxin drive system survives only if it inherits a copy of each of antitoxin A and antitoxin B. In some embodiments, (a) the first DNA cassette is inserted into the genome and comprises sequences encoding: (i) optionally one or more cargo genes, (ii) an antitoxin in the form of a copy of a recoded target gene B that when expressed functions to rescue the embryo's reproductive potential in the absence of other functional copies of target gene B, and (iii) a toxin in the form of a CRISPR nuclease and one or more guide RNAs that when expressed in the organism cut and disrupt target gene B, which is a gene required for one or both of viability and fertility of the organism as needed so the organism is able to reproduce, and (b) the second DNA cassette is inserted into an unlinked locus in the genome and comprises sequences encoding (i) optionally one or more cargo genes, (ii) an antitoxin in the form of a copy of a recoded target gene A that when expressed functions to rescue the embryo's reproductive potential, and (iii) a toxin in the form of a CRISPR nuclease and one or more guide RNAs that when expressed in the organism cut and disrupt the target gene B, which is a gene required for organism to reproduce, wherein only an offspring of the organism comprising the toxin-antitoxin drive system that inherits a copy of each antitoxin a and antitoxin B is able to reproduce. In some embodiments, the first DNA cassette and not the second DNA cassette comprises a sequence encoding a CRISPR nuclease, and wherein the second DNA cassette comprises DNA encoding one or more guide RNAs that function with the CRISPR nuclease. In some embodiments, the target genes A and B are required for the embryo to become a fertile adult organism. In some embodiments, the target genes A and B are required for the embryo to be viable. In some embodiments, the target genes A and B are required for the organism to be fertile. In some embodiments, the toxin-antitoxin system comprises a killer-rescue gene drive system, wherein a first DNA cassette inserted into the genome encodes a recoded copy of target gene A sufficient to rescue organism viability or fertility, and a second DNA cassette inserted into the genome encodes a CRISPR nuclease and one or more guide RNAs expressed so as to cut and disrupt wild-type copies of target gene A. In some embodiments, the toxin-antitoxin system comprises a threshold-dependent Medea gene drive system, wherein a first DNA cassette inserted into the genome comprises a sequence encoding a recoded copy of a target gene A sufficient to rescue embryo viability in the absence of other functional copies and further comprises a sequence encoding a CRISPR nuclease and one or more guide RNAs that when expressed function in the embryo organism and cut and disrupt all wild-type copies of target gene A. In some embodiments, at least one functional copy of the target genes A and B is required for the embryo to become a fertile adult organism. In some embodiments, at least one functional copy of the target genes A and B is required for the embryo to be viable. In some embodiments, at least one functional copy of the target genes A and B is required for the organism to be fertile. According to an aspect of the invention, one or more of an organism or population of organisms comprising a toxin-antitoxin gene drive system is provided. In some embodiments, the organism or a plurality of the organism is released into the wild.

According to an aspect of the invention, methods of constructing a gene drive system that combines nuclease-induced copying with threshold-dependence are provided, the methods including, the method comprising: (a) inserting into a genome one or more first DNA cassettes, wherein the first DNA cassettes comprises sequences encoding one or more components of a threshold-dependent gene drive system, and (b) inserting into the genome one or more second DNA cassettes, wherein the second DNA cassettes comprises sequences encoding one or more components of a nuclease-based gene drive system, wherein the nuclease-based drive system is designed to cut one or more target DNA sequences in at least one germline cell of a heterozygote organism resulting in copying of the one or more first DNA cassettes, and wherein the first DNA cassettes optionally further comprise sequences encoding one or more cargo genes. In some embodiments, the method also comprises including a recombinase and exchanging genes. In some embodiments, none of the components of the nuclease-based gene drive system are copied by the action of the nuclease-based gene drive system. In some embodiments, all components of the nuclease-based gene drive system are copied by the action of the nuclease-based gene drive system. In some embodiments, some but fewer than all components of the nuclease-based gene drive system are copied by the action of the nuclease-based gene drive system. In some embodiments, at least one nuclease involved in the nuclease-based gene drive system is an RNA-guided DNA-binding protein nuclease, and optionally is a CRISPR nuclease. In some embodiments, the nuclease-based drive system components comprise a daisy-chain gene drive. In some embodiments, the nuclease-based drive system components comprise a daisyfield gene drive. In some embodiments, none of the components of the nuclease-based drive system are affected by the action of the threshold-dependent gene drive system. In some embodiments, one or more of the components of the nuclease-based drive system are affected by the action of the threshold-dependent gene drive system. In some embodiments, one or more nuclease genes are affected by the action of the threshold-dependent gene drive system. In some embodiments, nuclease genes are not affected by the action of the threshold-dependent gene drive system. In some embodiments, the threshold-dependent gene drive system is a toxin-antitoxin system. In some embodiments, the toxin-antitoxin system is based on an RNAi toxin. In some embodiments, the toxin-antitoxin system is based on a CRISPR toxin. In some embodiments, the threshold-dependent gene drive system is a Medea system. In some embodiments, the threshold-dependent gene drive system is the result of a chromosomal translocation generated as a consequence of the DNA cassette insertion. In some embodiments, the threshold-dependent gene drive system comprises two or more haploinsufficient or nearly haploinsufficient genes that have exchanged places. In some embodiments, the nuclease-based gene drive system cuts the wild-type haploinsufficient genes, and the haploinsufficient genes that have exchanged places have been recoded so as to not be cut, wherein the recoding comprises changing the bases of the gene without changing the resulting protein, and cutting results in copying each of the recoded haploinsufficient genes. In some embodiments, the method also includes: (f) sampling a target population of the wild-type organism strain and estimating the number of organisms; (g) releasing a number of the complete combined gene drive strain organisms of step (e) at least sufficient to edit a portion of the genome of at least a portion of the target population; (h) sampling strains of organisms collected from the target population following the release and confirming that a suitable fraction of the target population has been edited; (i) releasing additional daisy drive or wild-type organisms to adjust the boundaries of the edited population as desired; and optionally (j) releasing organisms encoding one or more suppressor elements into the target population, wherein the suppressor elements will spread through and reduce the fertility of organisms that were edited by the release in step (g), but not wild-type organisms. In some embodiments, the suppressor element(s) disrupts one or more recessive viability, fertility, sex-specific fertility, or female-specific fertility genes in germline cells of affected organisms. In some embodiments, the suppressor element(s) distort the sex ratio. In some embodiments, the nuclease-based gene drive system is present in the genome of an organism and comprises a CRISPR multiplex system, wherein at least one of the second DNA cassettes comprises one or more guide RNAs of self-processing CRISPR system and at least one other of the second DNA cassettes comprises one or more guide RNAs of a non-self-processing CRISPR system, and wherein a nuclease from the self-processing CRISPR system and a nuclease from the non-self-processing CRISPR system are each expressed in the organism. In some embodiments, the self-processing CRISPR system is a Cpf1 system and the non-self-processing CRISPR system is a Cas9 system. According to an aspect of the invention, one or more of an organism or population of organisms comprising a gene drive system that combines nuclease-induced copying with threshold-dependence is provided. In some embodiments, the organism or a plurality of the organism is released into the wild.

According to an aspect of the invention, methods of generating engineered underdominance in a population of organisms are provided, the methods including exchanging in one or more organisms in the population, the positions of a first haploinsufficient gene on a first chromosome in a cell of the one or more organisms with a second haploinsufficient gene on a second chromosome in the cell of the one or more organisms. In some embodiments, the first and second haploinsufficient genes are ribosomal genes. In some embodiments, neither the first nor the second haploinsufficient genes are ribosomal genes. In some embodiments, wherein only one of the first and the second haploinsufficient genes is a ribosomal gene. In some embodiments, the cell is a zygote. In some embodiments, the cell is a gamete. In some embodiments, the cell can give rise to a gamete.

According to an aspect of the invention, methods of generating engineered toxin-antitoxin underdominance in a population of organisms are provided, the methods including: preparing an active CRISPR system that targets and disrupts one or more essential or haploinsufficient genes and provides an antidote in the form of one or more recoded copies of the haploinsufficient, wherein only offspring that inherit a copy of each of the one or more antidotes survive and including the active CRISPR system in at least one cell of one or more organisms in a population. In some embodiments, the at least one cell is a zygote. In some embodiments, the active CRISPR system targets and disrupts 1, 2, 3, 4, 5, 6, 7, 8, or more independently selected haploinsufficient genes. In some embodiments, the target gene comprises at least one of: a large ribosomal subunit gene and a small ribosomal subunit gene. In some embodiments, two or more haploinsufficient genes are disrupted and equivalent functional copies of the antidote are encoded with the cargo element. In some embodiments, the engineered toxin-antitoxin gene drive system is an engineered Medea-class toxin-antitoxin gene drive system. In some embodiments, a means of disrupting comprises encoding a nuclease that is expressed in the germline of the organism wherein the nuclease cuts the haploinsufficient target gene(s) at one or more locations and wherein the functional copies encoded with the cargo element are recoded by changing one or a plurality of nucleic acid bases in the gene such that the gene is not cut by the nuclease without changing the amino acid sequence of the resulting protein. In some embodiments, a means of disrupting further comprises including a new 3′UTR in one or more cargo elements. In some embodiments, a nuclease used for one or both of disruption and encoding is an RNA-guided DNA-binding protein nuclease. In some embodiments, the target gene is a haploinsufficient gene. In some embodiments, the target gene is not a haploinsufficient gene.

According to another aspect of the invention, methods of preparing a precision underdominant daisy chain gene drive system are provided, the methods including (a) selecting a gene drive system; (b) identifying one or more target haploinsufficient genes of an organism in which the gene drive system components and cargo elements will be included; (c) constructing the gene drive system by: (i.) recoding one or more of the identified target genes, wherein the recoding comprises changing the bases of the gene without changing the resulting protein with or without including a new 3′UTR for each identified target gene in a gene drive cassette; (ii.) swapping the positions of the haploinsufficient genes in the daisy drive cargo elements such that all offspring of an organism that includes the precision underdominant daisy chain gene drive inherit one copy of the recoded version of each haploinsufficient gene only when the gene drive is active, wherein underdominance results in progeny of the organism that only inherit the daisy drive cargo elements without any other daisy drive elements; (d) preparing one or more organism strains each comprising one of the constructed precision underdominant daisy chain gene drive systems of step (c); and (e) crossing a prepared organism strain of (d) with an N−1 daisy chain gene drive strain of the organism and homozygosing offspring of the crossing, wherein offspring of the crossing are complete (N) daisy chain gene drive strain organisms. In some embodiments, the method also includes (f) sampling a target population of the wild-type organism strain and estimating the number of organisms; (g) releasing a number of the complete (N) daisy chain gene drive strain organisms of step (e) at least sufficient to recode a portion of the genome of at least a portion of the target population; (h) sampling strains of organisms collected from the target population following the release and confirming that a suitable fraction of the target population has been recoded; (i) releasing additional daisy drive or wild-type organisms to adjust the boundaries of the recoded population as desired; and optionally (j) releasing organisms of suppressor daisy chain gene drive strain of prepared in step (f) into the target population, wherein the daisy chain gene drive will spread through and suppress the organisms of the population that were recoded by the release in step (g), but not the wild-type organisms. In some embodiments, the gene drive system is based on an RNA-guided DNA-binding protein nuclease. In some embodiments, a penultimate daisy drive element in the gene drive system comprises a nuclease and each cargo element of the gene drive system has one or more guide RNAs targeting the wild-type haploinsufficient gene in the other locus. In some embodiments, each cargo element in the gene drive system has one or more guide RNAs targeting its own locus. In some embodiments, wherein the gene drive system comprises one or more additional daisy drive elements each comprising a guide RNA targeting the next locus in the daisy drive chain such that the final of the additional daisy drive elements targets the penultimate daisy drive element that encodes the nuclease. In some embodiments, the daisy drive elements optionally encode a recoded haploinsufficient gene in which one or a plurality of bases have been changed such that they are not cut by the nuclease without changing the amino acid sequence of the resulting protein, and with or without including a new 3′UTR for each identified target gene in a gene drive cassette.

According to an aspect of the invention, methods of preparing a precision toxin-antitoxin daisy chain gene drive system are provided, the methods including: (a) selecting a gene drive system; (b) identifying one or more target essential or haploinsufficient genes of an organism in which the gene drive system will be included; (c) constructing a daisy chain drive in the gene drive system, wherein the daisy chain drive comprises one of an RNAi-based toxin-antitoxin locus incorporated in a cargo element of the daisy chain drive and other of the RNAi-based toxin-antitoxin locus incorporated into another element of the daisy chain drive, wherein the toxin disrupts the target essential or haploinsufficient gene; (d) preparing one or more organism strains each comprising the constructed daisy chain gene drive systems of step (c); and (e) crossing a prepared organism strain of (d) with an N−1 daisy chain gene drive strain of the organism and homozygosing offspring of the crossing, wherein offspring of the crossing are complete (N) daisy chain gene drive strain organisms, wherein as long as the constructed daisy chain gene drive is active, underdominance will not occur in offspring from the daisy chain gene drive strain organism and when the constructed daisy chain gene drive is not active, underdominance will occur. In some embodiments, the one of the RNAi-based toxin-antitoxin locus is the toxin locus and the other of the RNAi-based toxin-antitoxin locus is the antitoxin locus. In some embodiments, the toxin is a zygotically active version of CRISPR that disrupts an essential or haploinsufficient gene and the antitoxin consists of one or more recoded copies of the toxin locus. In some embodiments, the constructed daisy chain gene drive comprises two cargo elements carrying a zygotically active CRISPR nuclease and guide RNAs targeting a haploinsufficient gene as well as a recoded copy of the targeted gene, wherein only offspring that inherit a copy of each of the two cargo elements survive. In some embodiments, the constructed daisy chain gene drive comprises a plurality of cargo elements and encodes a zygotically active CRISPR nuclease that targets a identified target haploinsufficient gene and also encodes a recoded version of one or more identified target genes wherein only offspring that inherit a copy of each of the plurality of cargo elements survive. In some embodiments, the toxin is a zygotically active form of RNAi and the antidote consists of one or more recoded copies of at least one of the identified target genes. In some embodiments, the toxin is a maternally active form of RNAi and the antidote consists of one or more recoded copies of at least one of the identified target genes.

According to another aspect of the invention, methods of preparing an engineered organism are provided, the methods including: inserting a preselected DNA sequence into a plurality of repeated regions in the genome of an organism of a strain to prepare a first engineered organism, wherein a means of inserting the preselected DNA sequence comprises: (a) delivering into a cell of the organism, a gene cassette encoding one or more guide RNAs; (b) inserting the gene cassette into a plurality of repeated regions in the genome of the organism to prepare an engineered organism; and (c) expressing the one or more guide RNAs, wherein in the presence of an RNA-guided protein nuclease in the cell the expressed guide RNAs direct cutting of a target DNA sequence on a chromosome of the organism. In some embodiments, the method also includes repeating the insertion of the preselected DNA sequence into a plurality of repeated regions in the genome in a plurality of organisms of the strain to prepare a plurality of the first engineered organisms. In some embodiments, the method also includes releasing one or a plurality of the first engineered organisms into a population comprising one or more non-engineered organisms of the strain. In some embodiments, the population is a wild population. In some embodiments, the cutting of the target DNA sequence stimulates copying of a genetic element on a sister chromosome of the chromosome in place of the cut target sequence. In some embodiments, the copied genetic element encodes the RNA-guided protein nuclease. In some embodiments, a means for inserting the gene cassette comprises sequence-directed nuclease insertion or recombinase insertion. In some embodiments, a means for inserting the gene cassette comprises CRISPR-based methods. In some embodiments, the means for inserting the gene cassette comprises use of one or more: transposons or retrotransposons. In some embodiments, the cell is one or more of: a zygote, a gamete, and a cell that gives rise to a gamete. In some embodiments, wherein the cassette also includes a promoter/enhancer/3′UTR sequence. In some embodiments, the cassette also includes a sequence encoding an RNA-guided DNA nuclease positioned downstream of the 3′UTR. In some embodiments, the promoter is a: U6, H1, 7SK, Pol II, or Pol III promoter. In some embodiments, the target DNA sequence comprises at least a portion of a ribosomal gene. In some embodiments, the target DNA sequence comprises at least a portion of a neutral gene. In some embodiments, the organism is a vertebrate. In some embodiments, the vertebrate is a rodent. In some embodiments, the organism is an invertebrate. In some embodiments, the organism is a strain of a: Rattus rattus, Aedes aegypti, Culex quinquefasciatus, or Anopheles gambiae. In some embodiments, the method also includes (d) sampling a target population of the organism strain that is not the engineered organism strain and estimating the number of organisms; (e) releasing a number of the engineered organisms at least sufficient to recode a portion of the genome of at least a portion of the target population; (f) sampling strains of organisms collected from the target population following the release of step (e) and confirming that a suitable fraction of the target population has been recoded; and (g) releasing additional of the engineered organisms to adjust the boundaries of the recoded population as desired. In some embodiments, the method also includes crossing the engineered organism with another strain of the organism. In some embodiments, the gene cassette additionally encodes an RNA-guided DNA nuclease downstream of the 3′UTR in gene cassette. In some embodiments, one or more of the guide RNAs comprises alternating Cas9 sgRNAs with CPf1 crRNAs.

According to another aspect of the invention, engineered organisms are provided, wherein the engineered organisms include a preselected DNA sequence inserted into a plurality of repeated regions in the organism's genome. In some embodiments, the organism comprises one or more CRISPR system components. In some embodiments, the preselected DNA sequence insertion comprises CRISPR-based methods. In some embodiments, (a) one or more cells of the organism comprise a gene cassette encoding one or more preselected guide RNAs; (b) the gene cassette is present in a plurality of repeated regions in the genome of the engineered organism; and (c) preselection of the one or more guide RNAs comprises selecting one or more guide RNAs that when expressed in a cell of the organism and in the presence of an RNA-guided protein nuclease in the cell, the one or more guide RNAs direct cutting of a target DNA sequence on a chromosome of the organism. In some embodiments, the cutting of the target DNA sequence stimulates copying of a genetic element on a sister chromosome of the chromosome in place of the cut target sequence. In some embodiments, the copied genetic element encodes the RNA-guided protein nuclease. In some embodiments, the cell is one or more of: a zygote, a gamete, and a cell that gives rise to a gamete. In some embodiments, in some embodiments, the cassette further comprises a promoter/enhancer/3′UTR sequence. In some embodiments, wherein the cassette also includes a sequence encoding an RNA-guided DNA nuclease positioned downstream of the 3′UTR. In some embodiments, the promoter is a: U6, H1, 7SK, Pol II, or Pol III promoter. In some embodiments, the target DNA sequence comprises at least a portion of a ribosomal gene. In some embodiments, the target DNA sequence comprises at least a portion of a neutral gene. In some embodiments, the organism is a vertebrate. In some embodiments, the vertebrate is a rodent. In some embodiments, the organism is an invertebrate. In some embodiments, the organism is a strain of a: Rattus rattus, Aedes aegypti, Culex quinquefasciatus, or Anopheles gambiae. In some embodiments, one or more of the preselected guide RNA comprises alternating Cas9 sgRNAs and CPf1 crRNAs.

According to another aspect of the invention, methods of preparing a gene-drive engineered organism are provided, the methods including: (a) selecting a gene drive system based on an RNA-guided DNA-binding protein nuclease; (b) delivering to a cell in an organism two or more independently selected gene cassette elements, wherein at least one of the gene cassette elements is an effector element; at least one of the gene cassette elements is a driving element, at least one of the driving elements drives the effector element, and each driving element gene cassette encodes one or more independently selected guide RNAs; (c) inserting the driving gene cassette element that drives the effector element into a plurality of repeated regions in the genome of the organism to prepare a gene-drive engineered organism; and (d) expressing the one or more guide RNAs of the driving gene cassette element, wherein in the presence of an RNA-guided protein nuclease in the cell the expressed driving gene cassette element guide RNAs direct cutting of a target DNA sequence on a chromosome of the organism, and the expressed driving gene cassette element drives the effector element to be copied in place of the target sequence. In some embodiments, the effector element encodes the RNA-guided protein nuclease. In some embodiments, the cutting of the target DNA sequence stimulates copying of a genetic element on a sister chromosome of the chromosome in place of the cut target sequence. In some embodiments, each of the gene cassettes elements comprises an independently selected sequence encoding a promoter/enhancer/3′UTR and one or more independently selected guide RNA sequences. In some embodiments, one or more of the gene cassette elements further comprises a sequence encoding an RNA-guided DNA nuclease positioned downstream of the 3′UTR sequence. In some embodiments, selecting the gene drive system comprises selecting a target gene of the driving gene cassette element. In some embodiments, the gene drive system is a CRISPR gene drive system. In some embodiments, the copied genetic element encodes the RNA-guided protein nuclease. In some embodiments, a means for inserting the driving gene cassette comprises sequence-directed nuclease insertion or recombinase insertion. In some embodiments, the sequence-directed nuclease insertion means or recombinase insertion means comprise one or more of: transposons, retrotransposons, or other broken elements. In some embodiments, a means for inserting the driving gene cassette comprises CRISPR-based methods. In some embodiments, the cell is one or more of: a zygote, a gamete, and a cell that can give rise to a gamete. In some embodiments, the promoter is a: U6, H1, 7SK, Pol II, or Pol III promoter. In some embodiments, the target DNA sequence comprises at least a portion of a ribosomal gene. In some embodiments, the target DNA sequence comprises at least a portion of a neutral gene. In some embodiments, the organism is a vertebrate. In some embodiments, the vertebrate is a rodent. In some embodiments, the organism is an invertebrate. In some embodiments, the organism is a strain of a: Rattus rattus, Aedes aegypti, Culex quinquefasciatus, or Anopheles gambiae. In some embodiments, the method includes preparing a plurality of the engineered organisms. In some embodiments, the method also includes releasing the one or plurality of the prepared engineered organism into a population comprising organisms of an un-engineered strain of the engineered organism. In some embodiments, the method also includes (e) sampling a target population of the wild, non-engineered organism strain and estimating the number of organisms; (f) releasing a number of the engineered organisms at least sufficient to recode a portion of the genome of at least a portion of the target population; (g) sampling strains of organisms collected from the target population following the release of step (f) and confirming that a suitable fraction of the target population has been recoded; and (h) releasing additional of the engineered organisms to adjust the boundaries of the recoded population as desired. In some embodiments, the method also includes crossing the engineered organism with another strain of the organism. In some embodiments, one or more of the independently selected guide RNA comprises alternating Cas9 sgRNAs and CPf1 crRNAs.

According to another aspect of the invention, compositions are provided that include a gene system capable of directing CRISPR complexes method of directing CRISPR complexes to multiple target sequences by expressing two or more genes encoding different CRISPR nucleases, at least one of which is capable of processing its own associated CRISPR RNA (crRNA) array, and also expressing one or more CRISPR array composed of guide RNAs for the two or more CRISPR nucleases arranged in an alternating sequence. In some embodiments, one or more of the encoded CRISPR nucleases comprises a Cpf1-class enzyme. In some embodiments, one or more of the encoded CRISPR nucleases comprises a Cas9-class enzyme. In some embodiments, the CRISPR RNA array is produced by a DNA cassette comprising one or more instances of: (i) an independently selected promoter sequence, (ii) an encoded array of guide RNAs that correspond to each of the two or more nucleases, wherein the encoded promoter sequences are positioned in the DNA cassettes upstream of the encoded guide RNA array, and wherein the guide RNAs are arranged in array such that processing of a CRISPR RNA (crRNA) by its corresponding nuclease results in the liberation of individual or pairs of guide RNAs from the array in a manner that under appropriate conditions, permits each guide RNA to bind its appropriate nuclease and form an active CRISPR complex. In some embodiments, the guide RNAs and the nucleases are not encoded in the same DNA cassettes. In some embodiments, the composition is in a cell. In some embodiments, the cell is one or more of: a zygote, a gamete, and a cell that gives rise to a gamete. In some embodiments, the cassette further comprises a promoter/enhancer/3′UTR sequence. In some embodiments, the promoter of the CRISPR RNA array is a: U6, H1, 7SK, Pol II, or Pol III promoter. In some embodiments, the array is positioned within an intron or a 5′ or 3′ untranslated region (UTR) of a gene. In some embodiments, the cell is in an organism. In some embodiments, the organism is a vertebrate. In some embodiments, the vertebrate is a rodent. In some embodiments, the organism is an invertebrate. In some embodiments, the organism is a strain of a: Rattus rattus, Aedes aegypti, Culex quinquefasciatus, or Anopheles gambiae.

According to another aspect of the invention, methods of preparing a quorum organism that exhibits genetic underdominance are provided, the methods including (a) selecting a first candidate haploinsufficient gene; (b) inserting into at least one cell a first and a second independently selected recombinase site into the chromosome comprising the first candidate haploinsufficient gene, wherein the inserted first and second independently selected recombinase sites flank the candidate haploinsufficient gene and associated expression signals; (c) selecting a second candidate haploinsufficient gene, wherein the second candidate haploinsufficient gene is positioned in an unlinked locus, such as on a different chromosome, relative to the first candidate haploinsufficient gene; (d) inserting into the at least one cell a third and a fourth independently selected recombinase site into the chromosome locus comprising the second candidate haploinsufficient gene, wherein the inserted third and fourth independently selected recombinase sites flank the second candidate haploinsufficient gene and associated expression signals. In some embodiments, the method also includes assessing presence and position of each of the first, second, third, and fourth recombinase sites. In some embodiments, assessing comprises amplification and sequencing methods, and optionally wherein the amplification method comprises a polymerase chain reaction method. In some embodiments, the method also includes contacting each of the first, second, third, and fourth independently selected inserted recombinase sites with a recombinase specific for the first, second, third, and fourth recombinase site, respectively, under suitable conditions for recombination activity at the contacted recombinase sites, such that the two haploinsufficient genes exchange positions. In some embodiments, the method also includes determining the presence and positions of the first and second haploinsufficient genes. In some embodiments, the determining comprises amplification and sequencing methods, and optionally the amplification method comprises a polymerase chain reaction method. In some embodiments, the cell is in an organism. In some embodiments, the method also includes (a) crossing the organism comprising the inserted recombinase sites with a wild-type of the organism; and (b) assessing the status of the recombinase activity and underdominance in the organism. In some embodiments, the assessing comprises one or more of: quantifying the number of offspring from the cross, the presence and position of one or more of the first and second candidate haploinsufficient genes in the offspring. In some embodiments, the assessing comprises one or more of amplification methods, hybridization methods, and the use of detectable labels or marker gen3es inserted adjacent to one or more of the haploinsufficient genes. In some embodiments, two of the independently selected recombinase sites are mutually compatible, and optionally, the four independently selected recombinase sites comprise two of one type of recombinase site and two of another type of recombinase site.

According to another aspect of the invention, methods of preparing a quorum system that exhibits genetic underdominance are provided the methods including: (a) selecting a first candidate haploinsufficient gene positioned in a first chromosome; (b) inserting a first and a second independently selected recombinase site into the first chromosome, wherein the inserted first and second independently selected recombinase sites flank the first candidate haploinsufficient gene and relevant expression signals, and the chromosome is in a cell of a first organism; (c) selecting a second candidate haploinsufficient gene positioned in an unlinked locus such as on a second chromosome; (d) inserting a third and a fourth independently selected recombinase site into the second locus, wherein the inserted third and fourth independently selected recombinase sites flank the second candidate haploinsufficient gene and relevant expression signals, and the chromosome is in a cell of a second organism; (e) crossing the first organism with the second organism to prepare an engineered organism; and (f) contacting in an engineered strain organism each of the first, second, third, and fourth independently selected inserted recombinase sites with a recombinase specific for the first, second, third, and fourth recombinase site, respectively, under suitable conditions for recombination activity at the contacted recombinase sites that will exchange the positions of the first and second candidate haploinsufficient genes. In some embodiments, the method also includes assessing the status of the recombinase activity and underdominance in the engineered organism. In some embodiments, the assessing comprises one or more of: quantifying the number of offspring from the cross, the presence and position of one or more of the first and second candidate haploinsufficient genes in the offspring of the cross. In some embodiments, the method also includes (a) crossing an engineered organism with a wild type of the organism and (b) assessing the engineered organism strain for underdominance. In some embodiments, the assessing comprises one or more of: offspring viability determination methods, amplification methods, hybridization methods, and the use of detectable labels such as one or more marker genes inserted adjacent to one or more of the candidate haploinsufficient genes. In some embodiments, two of the independently selected recombinase sites are the same mutually compatible for recombination in the presence of the appropriate recombinase, and optionally, the four independently selected recombinase sites comprise two of one type of recombinase site and two of another type of recombinase site. In some embodiments, preparing the recombinase site insertion comprises CRISPR-based methods. In some embodiments, (a) one or more cells of the first and second organisms comprise a gene cassette encoding one or more preselected guide RNAs; (b) the gene cassette is present in a plurality of repeated regions in the genome of the engineered organism; and (c) preselection of the one or more guide RNAs comprises selecting one or more guide RNAs that when expressed in a cell of the engineered organism and in the presence of an RNA-guided protein nuclease in the cell, the one or more guide RNAs direct cutting of a target DNA sequence on a chromosome of the engineered organism. In some embodiments, the cutting of the target DNA sequence stimulates copying of a genetic element on a sister chromosome of the chromosome in place of the cut target sequence. In some embodiments, the copied genetic element encodes the RNA-guided protein nuclease. In some embodiments, the cell is one or more of: a zygote, a gamete, and a cell that gives rise to a gamete. In some embodiments, the cassette also includes a promoter/enhancer/3′UTR sequence. In some embodiments, the cassette also includes a sequence encoding an RNA-guided nuclease positioned downstream of the 3′UTR. In some embodiments, the promoter is a: U6, H1, 7SK, Pol II, or Pol III promoter. In some embodiments, the target DNA sequence comprises at least a portion of a ribosomal gene. In some embodiments, the target DNA sequence comprises at least a portion of a neutral gene. In some embodiments, the organism is a vertebrate. In some embodiments, the vertebrate is a rodent. In some embodiments, the organism is an invertebrate. In some embodiments, the organism is a strain of a: Rattus rattus, Aedes aegypti, Culex quinquefasciatus, or Anopheles gambiae.

According to another aspect of the invention method of preparing an engineered cell are provided, the method including: one or more of: (a) delivering into a cell the composition of any one of claims L1-L8; and expressing at least two of the two or more DNA cassettes, wherein one of the expressed DNA cassettes is the cassette comprising the gene that when expressed processes its associated CRISPR RNA (crRNA) array, and wherein expressing the DNA cassettes directs two or more CRISPR proteins to one or more of: (i) binding and (ii) cleaving multiple target DNA sequences, and (b) delivering into a cell the expressed product of at least two of the two or more DNA cassettes, wherein one of the expressed DNA cassettes is the cassette comprising the gene that when expressed processes its associated CRISPR RNA (crRNA) array, and wherein expressing the DNA cassettes directs two or more CRISPR proteins to one or more of: (i) binding and (ii) cleaving multiple target DNA sequences. In some embodiments, the method also includes delivering the composition into the cell and inserting the gene cassette into a plurality of repeated regions in the genome of the cell. In some embodiments, the cell is in an organism and the insertion of the gene cassette comprise insertion in to a plurality of repeated regions in the genome of the organism. In some embodiments, the organism is a vertebrate. In some embodiments, the vertebrate is a rodent. In some embodiments, the organism is an invertebrate. In some embodiments, the organism is a strain of a: Rattus rattus, Aedes aegypti, Culex quinquefasciatus, or Anopheles gambiae. In some embodiments, the gene cassette additionally encodes an RNA-guided DNA nuclease downstream of the 3′UTR in gene cassette. In some embodiments, one or more of the guide RNAs comprises alternating Cas9 sgRNAs with Cpf1 crRNAs.

According to another aspect of the invention, methods of constructing a gene drive system are provided, the methods including one or more embodiments of any of the aforementioned aspects of the invention.

According to another aspect of the invention, methods of constructing a daisy field gene drive system are provided, the methods including one or more embodiments of any of the aforementioned aspects of the invention.

According to another aspect of the invention, cells and organisms that include one or more of any of the aforementioned embodiments of gene drive components such as, but not limited to: DNA cassettes and combinations of gene drive components as set forth above are provided.

According to another aspect of the invention, methods of constructing a gene drive system are provided, the methods including one or more embodiments of any of the aforementioned aspects of the invention.

According to another aspect of the invention, a gene drive strain is provided that includes one or more embodiments of any of the aforementioned compositions of the invention.

According to another aspect of the invention, an organism is provided that includes one or more embodiments of any of the aforementioned compositions, gene drives, and/or gene drive components of the invention. In some embodiments, a plurality of the organism is released into the wild.

### Brief Description of Certain of the Sequences

SEQ ID NO: 1 is an amino acid sequence of an S.
pyogenes Cas9 protein sequence et al., [Deltcheva
et al., Nature 471, 602-607 (2011)]:
MDKKYSIGLDIGTNSVGWAVITDEYKVPSKKFKVLGNTDRHSIKKNLIG
ALLFDSGETAEATRLKRTARRRYTRRICNRICYLQEIFSNEMAKVDDSF
FHRLEESFLVEEDKKHERHPIFGNIVDEVAYHEKYPTIYHLRKKLVDST
DKADLRLIYLALAHMIKFRGHFLIEGDLNPDNSDVDKLFIQLVQTYNQL
FEENPINASGVDAKAILSARLSKSRRLENLIAQLPGEKKNGLFGNLIAL
SLGLTPNFKSNFDLAEDAKLQLSKDTYDDDLDNLLAQIGDQYADLFLAA
KNLSDAILLSDILRVNTEITKAPLSASMIKRYDEHHQDLTLLKALVRQQ
LPEKYKEIFFDQSICNGYAGYIDGGASQEEFYKFIKPILEKMDGTEELL
VKLNREDLLRKQRTFDNGSIPHQIHLGELHAILRRQEDFYPFLKDNREK
IEKILTFRIPYYVGPLARGNSRFAWMTRKSEETITPWNFEEVVDKGASA
QSFIERMTNFDKNLPNEKVLPKHSLLYEYFTVYNELTKVKYVTEGMRKP
AFLSGEQKKAWDLLFKINRKVTVKQLKEDYFKKIECFDSVEISGVEDRF
NASLGTYHDLLKIIKDKDFLDNEENEDILEDIVLTLTLFEDREMIEERL
KTYAHLFDDKVMKQLKRRRYTGWGRLSRKLINGIRDKQSGKTILDFLKS
DGFANRNFMQLIHDDSLTFKEDIQKAQVSGQGDSLHEHIANLAGSPAIK
KGILQTVKVVDELVKVMGRHKPENIVIEMARENQTTQKGQKNSRERMKR
IEEGIKELGSQILKEHPVENTQLQNEKLYLYYLQNGRDMYVDQELDINR
LSDYDVDHIVPQSFLKDDSIDNKVLTRSDKNRGKSDNVPSEEVVKKMKN
YWRQLLNAKLITQRKFDNLTKAERGGLSELDKAGFIKRQLVETRQITKH
VAQILDSRMNTKYDENDKLIREVKVITLKSKLVSDFRKDFQFYKVREIN
NYHHAHDAYLNAVVGTALIKKYPKLESEFVYGDYKVYDVRKMIAKSEQE
IGKATAKYFFYSNIMNFFKTEITLANGEIRKRPLIETNGETGEIVWDKG
RDFATVRKVLSMPQVNIVKKTEVQTGGFSKESILPKRNSDKLIARKKDW
DPKKYGGFDSPTVAYSVLVVAKVEKGKSKKLKSVKELLGITIMERSSFE
KNPIDFLEAKGYKEVKKDLIIKLPKYSLFELENGRKRMLASAGELQKGN
ELALPSKYVNFLYLASHYEKLKGSPEDNEQKQLFVEQHKHYLDEIIEQI
SEFSKRVILADANLDKVLSAYNKHRDKPIREQAENIIHLFTLTNLGAPA
AFKYFDTTIDRKRYTSTKEVLDATLIHQSITGLYETRIDLSQLGGD.

### BRIEF DESCRIPTION OF THE DRAWINGS

FIG. 1 is a schematic diagram illustrating how CRISPR gene drives distort inheritance in a self-sustaining manner by converting heterozygotes into homozygotes in the germline.

FIG. 2A-B provides two schematic diagrams illustrating an embodiment of a daisy drive system. FIG. 2A illustrates that a daisy drive system consists of linear daisy chains of serially dependent drive elements. FIG. 2B illustrates that elements at the base of the daisy chain cannot drive and are successively lost over generations, limiting overall spread.

FIG. 3 is a schematic diagram showing the family tree of a C→B→A embodiment of a daisy drive over four generations if all organisms mate with wild-type. 14/16 F4 descendants will inherit A, versus 1/16 for a non-drive, 8/16 for a B→A split drive, and 16/16 for a global CRISPR gene drive.

FIG. 4A-C provides schematic diagrams showing family tree analysis. FIG. 4A shows results of analysis of a B→A split drive and FIG. 4B shows results of analysis of a C→B→A daisy drive. FIG. 4C is a graphical depiction of total alleles per generation for B→A through D→C→B→A daisy drives.

FIG. 5 is a schematic diagram illustrating that recombination events that move a guide RNA from one element to another could create a “daisy necklace” capable of self-sustaining global drive.

FIG. 6 provides a list of sequence-divergent guide RNAs that were designed, constructed, and assayed using the transcriptional activation reporter. The sequences shown are, from top to bottom, SEQ ID NOs: 3-34.

FIG. 7 is a schematic diagram illustrating that ensuring that NHEJ events that repair drive-induced double-strand breaks impair ability to progress through gametogenesis, which will be compensated for by other cells that are not so impaired, can select against potential drive-resistant alleles while reducing or eliminating the fitness cost of doing so because total gamete production should be nearly or completely equivalent to wild-type, or at least an organism with the same drive system components that does not target important genes.

FIG. 8 is a schematic diagram showing a daisy drive system consisting of a number of serially-dependent elements in which each element in the daisy chain causes the next element to drive. The daisy chain can be of any desired length so long as the total fitness cost is not prohibitive.

FIG. 9 is a schematic diagram showing a family tree depicting inheritance of a simple 3-element daisy drive system.

FIG. 10 provides graphs illustrating that by adding more elements to a daisy drive fewer organisms are required to be released in order for the terminal A element to reach fixation in a wild population.

FIG. 11A-C provides graphs indicating that the dynamics of a C→B→A embodiment of a daisy drive alleles depends on the seeding frequency and fitness costs. FIG. 11A shows that a daisy drive with 2% fitness cost per upstream element and 10% fitness cost for the final element, seeded at 1%, never approaches fixation. FIG. 11B shows that the same drive seeded at 5% would rapidly fix in a non-deterministic model. FIG. 11C shows that if the upstream elements cost 10% each, more organisms would need to be released.

FIG. 12A-B provides graphs of modelling data illustrating that the A element attains higher frequencies as daisy-chain length increases across a range of fitness costs per upstream element, assuming the final element has a fitness cost of 10%. FIG. 12A shows that when a population was seeded at a level of 5%, three element chains were sufficient for the A element to reach 99% frequency if the upstream elements have a low fitness cost (2%, left). As the cost increases to 5% (middle), four elements were required, and 10% cost precluded spread above roughly 80%. FIG. 12B illustrates that daisy drives with more elements require fewer organisms to be released in order for the A element to reach a frequency of 99%. Each homing event is assumed to occur with 95% efficiency.

FIG. 13A-B provides graphs illustrating that releasing new organisms in each generation enables faster spread and requires fewer organisms per release. FIG. 13A shows results indicting that three- four- or five-element daisy drives can spread constructs with upstream elements having fitness costs of 2% (left) or 5% (middle) to 99% frequency. Four- or five-element drives are sufficient when the upstream elements have higher (10%) fitness costs. FIG. 13B shows results indicating that repeated release at very low frequency (0.1%) is sufficient for spread of the final element to 99% frequency for upstream elements having fitness costs of 2% (left) or 5% (middle), while >1% repeated release is required for higher cost (10%) elements.

FIG. 14A-B provides a sequence and a graph of results identifying highly active sequence-divergent guide RNAs for SPCas9. FIG. 14A shows a ‘Wild-type’ sgRNA sequence (SEQ ID NO: 2) that was the template sequence used to generate candidate gRNAs. FIG. 14B shows results of activity assays illustrating relative activities of guide RNAs based on a dCas9-VPR transcriptional activator screen using a tdTomato reporter.

FIG. 15 is a schematic diagram showing a potential family tree of a C→B→A embodiment of a genetic load daisy drive for which the payload in the A element disrupts a female fertility gene. The C element is male-linked, ensuring that it does not suffer a fitness cost from the loss of female fertility. Mating events between two parents carrying the A element (boxed) often produce sterile female offspring that will suppress the population.

FIG. 16 is a schematic diagram showing a male daisy-drive lineage whose daughters are always sterile, which permits dominant population suppression by titrating the number of males released.

FIG. 17A-J provides schematic diagrams illustrating embodiments of underdominance, CRISPR-based killer rescue systems of the invention. FIG. 17A illustrates that underdominance is achieved by swapping the locations of two haploinsufficient genes, such as ribosomal genes. Half the offspring of heterozygotes will perish due to failure to inherit two copies of each gene. FIG. 17B illustrates a version of underdominance that is created by a daisy drive system, which encodes the germline-expressed nuclease in the B element and swaps haploinsufficient (ribosomal) genes located in the A and U elements. FIG. 17C illustrates a CRISPR-based killer-rescue system, also referred to as: a toxin-antitoxin system, generated by inserting a copy of a haploinsufficient gene next to the payload and disrupting the wild-type copy elsewhere in the genome. FIG. 17D illustrates a killer-rescue system generated by a daisy drive system, which encodes the germline-expressed nuclease in the B element, a recoded copy of the haploinsufficient gene along with the payload in the A element, and guide RNAs that disrupt the wild-type copy in the U locus. FIG. 17E illustrates a more powerful killer-rescue system for which heterozygotes produce fewer progeny that is generated by encoding two different copies of a haploinsufficient gene next to the payload and disrupting the wild-type copy. FIG. 17F illustrates that a stronger killer-rescue system can also be generated by a daisy drive system so that it manifests after the drive halts. FIG. 17G-I provides diagrams of family trees demonstrating the underdominance effect and possible limited spread caused by the killer-rescue/toxin-antitoxin system. FIG. 17J illustrates a CRISPR-based toxin-antitoxin system that generates a Medea effect: any offspring that do not inherit the Medea element perish due to lack of a haploinsufficient gene.

FIG. 18A-C provides schematic diagrams showing embodiments of daisy drive systems for local and temporary population editing (TA1). Daisy drives exhibit controlled geographic and spatial spread due to the serial loss of daisy elements over generations in the face of Mendelian inheritance and natural selection. FIG. 18A illustrates a C→B→A drive, in which B and A can drive but C does not. FIG. 18B illustrates an embodiment in which loss of C causes B to cease driving; its subsequent loss prevents the payload element A from driving and eventually be lost. FIG. 18C provides an example family tree.

FIG. 19A-B provides schematic diagrams illustrating that daisy immunizing reversal drives can enable perfect genetic remediation of unauthorized global drives (TA2+3). FIG. 19A illustrates an example of how a daisy drive platform is adapted to eliminate any global drive that uses an orthogonal CRISPR nuclease then restore wild-type genetics. FIG. 19A, illustrates an embodiment in which a daisy platform with an immunizing reversal payload is crossed to the global drive, and the daisy overwrites it without losing elements because the payload directs the global drive's nuclease to copy all daisy elements. FIG. 19B illustrates a situation that when crossed to wild-type, the daisy immunizes it against the global drive, but loses elements and so cannot spread indefinitely (not shown). Once the global drive is eliminated, the daisy stops. The subsequent spread of a costly or suppression payload will eliminate all daisy elements, restoring wild-type genetics. Inclusion of daisy underdominance (see FIG. 21) ensures restoration.

FIG. 20 provides a schematic diagram showing an embodiment of a daisyfield drive system. A parallel version of daisy drive involves adding many copies of “B” throughout the genome, which ensures “A” exhibits drive for longer while requiring fewer editing events.

FIG. 21A-B provides schematic diagrams and graphs illustrating underdominance and daisy drive. FIG. 21A illustrates that swapping the positions of two haploinsufficient genes results in underdominance: half the offspring fail to inherit one of each and die. FIG. 21B illustrates that a daisy drive system can spread this swap or equivalents through the population by ensuring that offspring inherit one of each copy. When it runs out of daisy elements, underdominance prevents engineered genes from mixing into wild populations.

FIG. 22A-B provides schematic diagrams illustrating an embodiment of experimentally determining daisy drive stability and metapopulation dynamics. FIG. 22A illustrates how a linear group of huge nematode cultures, each with hundreds of millions of worms and adjacent transfer each generation, can be used to test drive stability and dynamics in what may be the only organism with populations large and fast reproducing enough to predict stability and behavior in the wild. FIG. 22B shows an embodiment in which, for better resolution, a liquid-handling robot that performs transfers between adjacent nematode populations at arbitrary amounts and frequencies and is used to experimentally test arbitrarily complex models of linked populations. Embodiments of liquid-handling tools are also used to demonstrate underdominance-based control and immunizing reversal and genetic remediation of unwanted global drives.

FIG. 23 provides a schematic diagram of an embodiment of nuclease-mediated multiplex insertion and construction of a daisyfield drive system. The inset section of FIG. 23 illustrates a strategy for efficient two-step multiplex insertion of DNA cassettes. Large DNA cassettes are also referred to herein as insertion DNA cassettes.

FIG. 24 provides a schematic diagram of an embodiment of building and testing basic quorum. The diagram shows how selected candidate haploinsufficient genes are flanked with recombinase sites. FIG. 24 indicates that the location and presence of correct insertions can be assessed and verified using standard methods such as amplification methods (for example, PCR) and sequencing). FIG. 24 also illustrates the effect of adding a recombinase, which results in swapping of the genes. The completion of the expected swap can be verified using standard methods such s amplification and sequencing methods. FIG. 24 illustrates crossing of a prepared engineered organism with a wild-type version of the organism and the expected results from such a cross. FIG. 24 indicates various types of assay methods that can be performed to determine the efficacy of the basic quorum.

FIG. 25A-B provides a schematic diagram of methods of building an embodiment of a quorum system of the invention and also including daisy drive components in the quorum genes. FIG. 25A illustrates editing ribosomal genes, mating the organisms that include the edited genes, swapping (exchanging) the introduced DNA and testing quorum underdominance by mating the engineered organism to wildtype and assessing viability of their offspring. FIG. 25B illustrates adding in daisy drive components, for example, CRISPR to quorum genes along with guide RNAs to separate daisy elements. FIG. 25B shows results of inclusion of the daisy drive in heterozygote germline, and results of mating in the absence of daisy elements.

FIG. 26 is a schematic diagram showing three daisy links used to prepare the C. elegans daisy drive. Daisy link ‘A’ contained myo3-mCherry-unc54 UTR flanked by 500 bp of both 5′ and 3′ homology sites for Cku80. Daisy link B contained Pmyo2-GFP-unc54UTR and guides targeting Cku80. It was flanked by both 5′ and 3′ homology arms to fog2. EM-Hera: Daisy link C contained Prp1128+BFP+let-858 UTR+gRNA targeting fog-2.

FIG. 27A-C provides three scatter-plot representation of Cq values from the qPCR. The data groups are clearly separated with an average of ˜1.2 cycles separating the ‘Daisy’ and ‘Control’ groups, indicating the drive system was successfully copied due to cutting of the wild-type allele and repair by homologous recombination. FIG. 27A shows results for Daisy Element “A”, FIG. 27B shows results for Daisy Element “B”, and FIG. 27C shows results for Daisy Element “C”.

### DETAILED DESCRIPTION

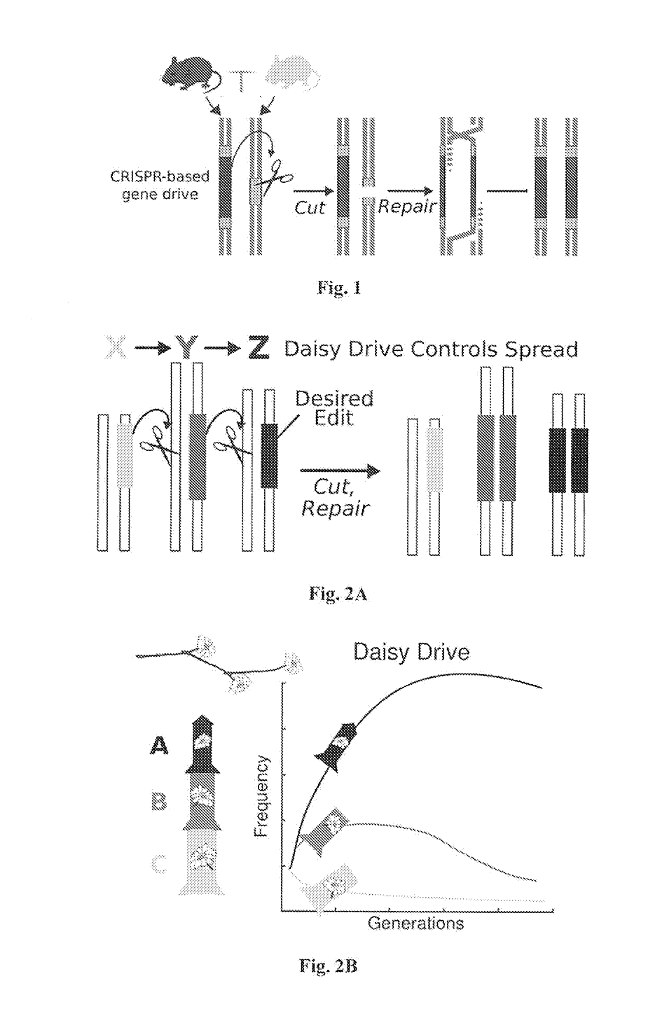

Gene drives are genome editing tools that can be used to spread selected genetic modifications through a targeted population of sexually reproducing organisms. Gene drives permit nucleic acid sequences to be introduced into cells, cells lines, and organism strains where they are directed to, and edit, a predetermined gene sequence. Gene drives are named for their ability to “drive” themselves and nearby genes through populations over many generations. Previous RNA-guided gene drive elements based on the CRISPR/Cas9 nuclease could be used to spread many types of genetic alterations through sexually reproducing species (Esvelt, K, et al., 2014 eLife:e03401) These gene drive elements function by “homing”, or the conversion of heterozygotes to homozygotes in the germline, which renders offspring more likely to inherit the gene drive element and the accompanying alteration than Mendelian inheritance would predict. FIG. 1 illustrates how global CRISPR gene drives distort inheritance in a self-sustaining manner by converting heterozygotes into homozygotes in the germline. The self-propagating nature of global gene drive renders the technology uniquely suited to addressing large-scale ecological problems, but tremendously complicates discussions of whether and how to proceed with any given intervention. In addition, there are currently few options for controlling unauthorized or accidentally-released global drive systems.

The invention, in part, relates to preparing and using types of gene drives that are designed to permit controlled, local gene drive activity. The novel control aspects allow release of a gene drive organism strain into a local population with the ability to confine the gene drive organisms such that they only affect local populations and do not risk global gene drive activities. Aspects of the invention, includes methods to design and construct powerful but locally-confined RNA-guided gene drive systems, that are designed to permit local containment of homing drives by arranging CRISPR-based drive components in an interdependent, daisy-chain-like manner, termed “daisy drives”.

The invention, in part, includes methods to design, construct and/or use embodiments of a “daisy chain gene drive”, which may also be referred to herein as gene drives or daisy drives. The invention, in part relates to methods of designing embodiments of daisy chain gene drive systems, for example, though not intended to be limiting: under-dominance embodiments and daisyfield embodiments, each of which may be used in embodiments of methods to modify and/or control local populations of organisms by implementation into local populations of organisms. Designing daisy chain drive systems and components thereof, may include one or more methods to select target genes, design, identify, and select active guide RNAs, identify promoter sequences, identify and use spacer sequences, design daisy chain drive elements, select tRNAs, select and use detectable labels, such as fluorescent detectable labels, etc. Certain aspects of the invention include combining one or more of the design and construction methods set forth herein and may also include delivering and implementing a daisy chain gene drive in a cell or organism strain. As used herein the term “daisy chain gene drive” means a gene drive that includes gene drive components configured in an interdependent, daisy-chain-like manner, termed “daisy drives”. In some embodiments of the invention a daisy chain gene drive is a CRISPR-based daisy chain gene drive and includes CRISPR-based drive components in an interdependent daisy chain configuration. FIG. 2 illustrates a general design strategy for certain embodiments of daisy chain gene drives. A daisy drive system of the invention consists of a linear series of genetic elements in which each element drives the next in the daisy chain. FIG. 2A illustrates one embodiment of a daisy chain drive that includes three elements, C→B→A. The final element in the chain (the “payload”) is driven to higher and higher frequencies in the population by the elements below it in the chain, much like the payload of a rocket is driven by the booster stages below (FIG. 2B). Because the element at the base of the daisy chain never exhibits drive, basal elements are progressively lost over generations. The more elements to a daisy drive, the higher the payload will be lifted.

### DETAILED DESCRIPTION

Methods of creating multi-locus assortment systems in a single step have now been identified. In addition, methods and compounds to prepare daisyfield drive systems in two steps have also been identified. The latter may be combined with underdominance for improved control over the extent of spread. Gene drives are genome editing tools that can be used to spread selected genetic modifications through a targeted population of sexually reproducing organisms. Gene drives permit nucleic acid sequences to be introduced into cells, cells lines, and organism strains where they are directed to, and edit, a predetermined gene sequence. Gene drives are named for their ability to “drive” themselves and nearby genes through populations over many generations. The self-propagating nature of gene drive renders the technology uniquely suited to addressing large-scale ecological problems such as parasite infestations, vector-transmitted disease outbreaks, etc.

Daisy drives work by harnessing Mendelian inheritance to programmably eliminate components until the drive is no longer active (Nobel, C. et al., (2016) bioRxiv 057307, doi:10.1101/057307). Daisy drives exhibit controlled geographic and spatial spread due to the serial loss of daisy elements over generations in the face of Mendelian inheritance and natural selection. FIG. 1a-c illustrates embodiments of a C→B→A drive, and shows the outcome when elements are lost. Because daisy drives rely on CRISPR genome editing, they can mimic any effect achievable with a standard RNA-guided gene drive system, but at a local and temporary scale, affording advantages for safety testing, local community decision-making, and regulation. In certain embodiments they can be used for perfect genetic removal of rogue genetic elements from populations (FIG. 2). Examining gene drive evolutionary dynamics in huge populations of nematodes allows empirical testing of drive system stability and safety over time.

Embodiments of drive systems are sensitive to various factors such as, but not limited to: homing efficiency, fitness cost, drive-resistant alleles, and recombinational instability. Inefficient homing is overcome by optimizing CRISPR expression and function, targeting multiple sites, and activating the drive in germline cells with a high homologous recombination rate. Fitness costs are minimized by targeting and recoding haploinsufficient genes to select against failed homing during gametogenesis, by using a “daisyfield design” in which many copies of guide-RNA-expressing daisy elements are inserted at repeated regions to minimize the number of homing events required, and/or by using daisy elements inserted at neutral genomic sites. Drive-resistant alleles are overcome by targeting many sites within genes important for fitness, which ensures that natural selection favors the drive system. Instability is overcome by avoiding sequence similarity through the use of different promoters and guide RNAs engineered for minimal similarity. Cost and effort are minimized by using predictive modeling and nematodes to test designs and by developing high-throughput transgenesis systems to accelerate the design-build-test cycle. Drive dynamics are predicted through mathematical modeling and empirical tests of spread, stability, and evolutionary behavior using very large populations of fast-reproducing nematode worms grown in flasks and small linked massively parallel populations with programmable gene flow rates maintained by a liquid-handling robot.

The invention, in part, relates to methods of using a sequence-directed nuclease or recombinase to insert a single DNA cassette into repeated regions present all over the genome of an organism, e.g. transposons, retrotransposons, or other broken elements; the cassette comprising at least one gene of interest. Certain aspects of the invention include methods and compositions that can be used to insert a plurality of copies of a gene sequence of interest into the genome of an organism, referred to herein as an “engineered organism”. Methods of the invention, in part, also include release of engineered organisms that include the plurality of copies of the DNA cassette containing one or more genes of interested that has been inserted into repeated regions throughout the genome of the engineered organism. Such methods of the invention can be used to spread that cassette efficiently into local wild populations of the organism, wherein offspring will inherit 50% of the parent's number of copies on average. In certain aspects of the invention, organisms arising from a local population in which engineered organisms have been released, will inherit one copy on average if the great-grandparent has 8 copies, or a great-great-grandparent has 16 copies, or a great-great-great grandparent has 32 copies, etc. This characteristic of the invention removes a prior limitation of needing to insert each element separately as a limiting factor.

As used herein the term “plurality” is used to mean at least two, and in certain aspects of the invention a plurality may be “two or more,” which may also be referred to herein as “at least two”; “three or more,” which may also be referred to herein as “at least three”, “four or more,” which may also be referred to herein as “at least four”. It will be understood that a plurality is may refer to at least 5, 10, 20, 30, 50, 50, 60, 70, 80, 90, 100, 200, 300, 400. As used herein in certain embodiments of the invention the term “plurality” refers to a number that is four or greater. Examples, though not intended to be limiting, include use of the term “plurality” in reference to the number of a first insertion cassette that is inserted throughout the genome of an organism, wherein plurality may mean a number that is four or greater; and use of the term “plurality” in reference to the number of organisms release into the wild, wherein plurality may mean a number that is 500, or 1,000, or 10,000 or larger.

Methods of the invention comprise inserting into repeated regions of an organisms genome many copies of a DNA cassette that encodes RNAs that in the presence of an RNA-guided protein nuclease direct the cutting of a target DNA sequence on a chromosome so as to stimulate copying of a genetic element on the sister chromosome in the place of the target sequence. In contrast to strategy used in some previous daisy drive methods, the aspects of the current invention comprise insertion of multiple copies of the basal element in the daisy-chain that direct cutting of the next element. In certain methods of the invention, the genetic element on the sister chromosome encodes the relevant nuclease. This results in what is referred to herein as pure “daisyfield” where a whole field of daisy elements is present and all act to cause a copy of the nuclease gene to drive, with half lost each generation on average, until the nuclease gene ceases to drive. It will be understood that as used herein, the term “nuclease” may also include other enzymes that cut single or double strands, for example a nickase may be considered a nuclease as used herein.

An example of a combination of daisy drive and daisyfield, though not intended to be limiting, is a situation in which there is one basal element, which is positioned on a Y chromosome to avoid the direct effects of a female-specific genetic load suppression drive, and the basal element targets the repeated elements in which the daisyfield elements are inserted, and those elements in turn drive the nuclease. In some aspects of the invention, an engineered organism is prepared that includes payload element(s) that cause underdominance, for example when there are two payloads that have swapped the positions of haploinsufficient genes such that half of progeny (on mating with wild-type) do not inherit one of each and consequently die.

It will be understood that although non-limiting embodiments of the invention are described as administering gene drive components in nucleic acid form, the invention also includes administering or delivering the gene drive components into a cell or organism in the form of polypeptides and/or expression products that have been prepared in vitro. In conjunction with the teaching provided herein, art known means can be used to prepare and utilize such expression products in methods, compositions, organisms, and organism strains of the invention.

### Daisyfield Gene Drive Systems

Methods of the invention, in part, include the use of one or more strategies to alter or suppress local populations of organisms, which in some embodiments of the invention, comprise wild populations of the organism. Daisyfield gene drives of the invention may be used for controlled, local gene drive activity. The novel control aspects allow release of a daisyfield gene drive engineered organism strain into a local population of the wild, non-engineered strain, with the ability to confine the daisyfield gene drive organisms such that they only affect local populations and do not risk global gene drive activities.

The invention, in part, includes methods to design, construct and/or use a novel type of gene drive, referred to as a “daisyfield gene drive”. The invention, in part relates to methods of designing daisyfield gene drive systems and methods to modify and/or control local populations of organisms by implementing daisyfield gene drive systems of the invention into local populations of organisms. Designing daisyfield drive systems and components thereof, may include one or more methods to select target genes, design, identify, and select active guide RNAs, identify promoter sequences, identify and use spacer sequences, design daisyfield drive elements, select tRNAs, select and use detectable labels, such as fluorescent detectable labels, etc. Certain aspects of the invention include combining one or more of the design and construction methods set forth herein and may also include delivering and implementing a daisyfield gene drive in a cell or organism strain.

As used herein the term “daisyfield gene drive” means a gene drive that includes gene drive components configured in an interdependent, daisyfield-like manner, termed “daisyfield drives”. In some embodiments of the invention a daisyfield gene drive is a CRISPR-based daisyfield gene drive and includes CRISPR-based drive components in an interdependent daisyfield configuration.

A daisyfield drive system of the invention consists of inserting into a plurality of a DNA regions of an organism's genome many copies of a DNA cassette that encodes RNAs that in the presence of an RNA-guided protein nuclease direct the cutting of a target DNA sequence on a chromosome so as to stimulate copying of a genetic element on the sister chromosome in the place of the target sequence.

### Daisy Chain Drive Systems

A daisy chain drive system designed using one or more methods of the invention can recapitulate any effect accessible to a global CRISPR gene drive, including either alteration or suppression. A daisy chain drive designed, constructed, and/or implemented using one or more methods of the invention, permits the spread of a terminal gene drive element “A” to be enhanced by including additional elements to the daisy chain of gene drive components. For example, but not intended to be limiting, a gene drive including elements C→B would be enhanced by adding element “A” to form daisy chain gene drive: C→B→A. Family tree analysis indicates that with such a gene drive design there will be many more copies of A relative to those generated using a previous gene drives designs, such as B→A split drives.

Aspects of the invention are based, in part, on the design and construction of daisy chain gene drives, and their use in cells, cell lines, and organisms as nuclease-based evolutionarily stable gene drive systems that are capable of altering or suppressing populations of organisms. Certain embodiments of daisy chain gene drives designed and prepared using methods of the invention include RNA-guided DNA binding proteins that when expressed in a cell co-localize with guide RNA at a target DNA site and act as gene drives. Daisy chain gene drive systems of the invention may be used to edit the genome of a host (target) cell or organism into which components of the daisy chain gene drive are delivered. As used herein, the terms “used” and “implemented” when used in reference to daisy chain gene drives, means a designed and constructed daisy chain gene drive is included in a cell or organism strain. It will be understood that implementation of a daisy chain gene drive may occur in one event or may be a multi-part implementation.

A daisy chain gene drive system that may be designed, constructed, and implement using one or more methods of the invention, is an RNA-guided DNA-binding protein endonuclease daisy chain gene drive system. Components of gene drive systems (for example: drive elements, guide RNAs, expression cassettes, vectors, endonucleases, promoters, DNA binding proteins, etc.) and methods for preparing and using such components, are known in the art and may be used in conjunction with methods of the invention to design, construct, and implement daisy chain gene drives of the invention, see for example: DiCarlo, J. E. et al., Nat Biotechnol. 2015 December; 33(12):1250-1255; Gantz V. M. & E. Bier Science, 2015 Apr. 24; 348(6233):442-4.; Gantz V. M. et al., Proc Natl Acad Sci USA. 2015 Dec. 8; 112(49); and Hammond, A. et al., Nat Biotechnol. 2015 Dec. 7; doi:10.1038/nbt.3439; the content of each of which is incorporated by reference herein in its entirety. In addition, methods and components of split-drive gene drives are known in the art and may be used in conjunction with methods described herein to design, construct, and implement daisy chain gene drives of the invention, see for example: Esvelt K. et al., eLife 2014; 3:e03401, the content of which is incorporated by reference herein in its entirety.

Embodiments of certain RNA-guided DNA-binding protein endonuclease daisy chain gene drive systems of the invention include aspects of CRISPR systems. Details of CRISPR systems such as CRISPR-Cas systems and examples of their use are known in the art, see for example: Deltcheva, E. et al. Nature 471, 602-607 (2011); Gasiunas, G., et al., PNAS USA 109, E2579-2586 (2012); Jinek, M. et al. Science 337, 816-821 (2012); Sapranauskas, R. et al. Nucleic acids research 39, 9275-9282 (2011); Bhaya, D., et al., Annual review of genetics 45, 273-297 (2011); and H. Deveau et al., Journal of Bacteriology 190, 1390 (February, 2008), the content of each of which is incorporated by reference herein in its entirety.

Three classes of CRISPR systems are generally known and are referred to as Type I, Type II or Type III. According to one aspect of the invention, methods to design and/or construct a daisy chain gene drive may include features of one or more of the three classes of CRISPR systems. Type I, II, and III CRISPR systems and their components are well known in the art. See for example, K. S. Makarova et al., Nature Reviews Microbiology 9, 467 (June, 2011); P. Horvath & R. Barrangou, Science 327, 167 (Jan. 8, 2010); H. Deveau et al., Journal of Bacteriology 190, 1390 (February, 2008); J. R. van der Ploeg, Microbiology 155, 1966 (Jim, 2009), the contents of each of which is incorporated by reference herein in its entirety. Bioinformatic analyses have generated extensive databases of CRISPR loci in a variety of bacteria that maybe used in conjunction with methods of the invention to design and construct daisy chain gene drives. See for example: M. Rho, et al., PLoS genetics 8, e1002441 (2012) and D. T. Pride et al., Genome Research 21, 126 (January, 2011) each of which is incorporated by reference herein in its entirety. A recently designated Type V system is similar in many aspects to Type II systems and may be relevant for genome editing and therefore gene drive systems (B. Zetsche et al., 2015, Cell 163, 1-13; T. Yamano et al., 2016, Cell, April 21 doi:10.1016/j.ce11.2016.04.003; D. Dong et al., 2016, Nature, 20 April, doi:10.1038/nature17944; I. Fonfara et al., 2016, Nature, 20 April, doi:10.1038/nature17945). It will be understood that references herein to “Cas9”, the RNA-guided DNA-binding protein nuclease of Type II CRISPR systems, can be replaced by “Cpf1”, the RNA-guided DNA-binding protein nuclease of Type V systems. It will be understood, as described elsewhere herein, certain embodiments of daisy chain gene drives of the invention may include a targeted DNA-binding nuclease other than an RNA-guided DNA-binding nuclease. For example, in some embodiments a daisy chain gene drive may include a nucleic acid-guided DNA binding nuclease such as a DNA-guided DNA-binding nuclease (see Gao, F., et al., Nature Biotech online publication, May 2, 2016: doi:10.1038/nbt.3547, the content of which is incorporated herein by reference).

### Drive Systems General Strategies for Design, Construction, and Deployment

A daisy chain drive system includes a linear series or “chain” of genetic elements in which each element drives the next element in the daisy chain. A daisy chain drive system designed using methods of the invention can be introduced into a population of organisms and the “payload” or top element of the chain is driven to higher and higher frequencies in the population by the elements below it in the chain. In some embodiments of the invention, a payload or top element may be an effector element. As used herein, an effector element performs a function when it is driven. Because the element at the base of the daisy chain never exhibits drive, the base elements in the chain may be progressively lost over generations. The more elements to a daisy drive, the higher the frequency of the payload in the population. A non-limiting example of a daisy chain drive system is a drive that includes three genetic elements, and is represented as C→B→A. In the example daisy chain drive, the payload element is the “A” element and the element at the base of the chain is element “C”. It will be understood that additional elements, represented as elements D, E, F, G, etc. may be included in a daisy chain drive designed and constructions using methods of the invention, non-limiting examples of which are daisy drives D→C→B∝A, E→D→C→B→A, and F→E→D→C→B→A, etc. As used herein, the letters A, B, C, D, E, F, etc. each represents a different element in a daisy chain designed and/or constructed using methods such as those disclosed herein.

It has now been identified that the spread of an element A in a gene drive may be enhanced by adding additional links in the daisy chain of gene drive components, e.g. C→B→A. Thus, inclusion of a daisy chain gene drive such as C→B→A in a population of an organism will result in many more copies of “A” relative to the number that would result from a inclusion of a B→A split drive. Assuming fitness neutrality and ignoring stochastic effects, a comparatively small release of organisms encoding an C→B→A daisy drive will result in a fixed incidence of C, increasing B, and a more rapidly increasing A. The “chain” of a daisy chain drive can be extended indefinitely, e.g. D→C→B→A etc. to achieve successively more powerful local drive. Thus in some aspects of the invention a daisy chain gene drive includes at least 3, 4, 5, 6, 7, 8, 9 10, 11, 12, 13, 14, 15, 16, 17, 18, 19, 20, or more elements. A daisy chain gene drive system may be designed using methods of the invention to include a linear series of genetic elements in which each element causes the one, immediately downstream to exhibit drive, for example, though not intended to be limiting: a daisy gene drive with elements D→C→B→A, of which the furthest upstream element is D and the furthest downstream element is A.

Certain embodiments of the invention include methods that can be used independently, or in combination with each other or with other gene drive design methods, to prepare daisy chain gene drives and daisyfield drives that can be used to construct powerful and locally-confined RNA-guided drive systems. Daisyfield gene drives and daisy chain gene drives designed using one or more methods of the invention, can be delivered into cells, cell lines, and/or organisms where they act to edit the genome in a stable, controlled manner. Daisyfield and Daisy chain drive systems designed using one or more methods of the invention may be utilized in stable genome-modifying applications for which global drive systems and/or existing local drives are unsuitable. For example, methods of the invention can be used to prepare one or more “daisyfield drive” and “daisy chain drive” organism strains that may then be released into a local wild population of the organism. The presence of the daisyfield drive organisms and/or daisy chain drive organisms as a predetermined small fraction of a local wild population of the organism can be used to drive a useful genetic element, included in the drive, to local fixation for a wide range of fitness parameters without resulting in global spread. It will be understood that Daisy chain gene drives designed using methods of the invention may permit local communities to decide whether, when, and how to alter shared regional ecosystems.

Methods of the invention, in some aspects, include design, construction, and use of daisy chain gene drives systems that include a “generic” daisy chain gene drive. As used herein, a generic daisy chain gene drive includes N−1 elements, where N is the total number of elements in the complete chain of the daisy chain gene drive system. In embodiments of an N−1 daisy chain gene drive, the terminal element in the chain (designated the B element) encodes an RNA-guided DNA nuclease and the “A” element is not included in the N−1 daisy chain gene drive. An N−1 daisy chain gene drive of the invention can be utilized in a number of different methods for modulating gene expression and organism populations. One non-limiting example is delivery into an organism that includes a “generic” N−1 daisy chain gene drive, an “A” element designed to accomplish a desired genome modulation (for example gene alteration or suppression) that also encodes guide RNAs that enable “A” to drive in the presence of the RNA-guided DNA nuclease (encoded in the “B” element). In this scenario, the “A” element may be added to the organism's genome directly by standard methods known to those in the art so as to create a complete N-element daisy chain drive organism, that is effective to accomplish the desired genome modulation. In another non-limiting example, a “generic” N−1 organism strain is prepared and another organism of the same species background is prepared that includes an “A” element designed to accomplish a desired genome modulation, such as gene alteration or suppression, and that also encodes guide RNAs that enable “A” drive in the presence of the RNA-guided DNA nuclease (encoded in the “B” element). The organism strain comprising the N−1 daisy chain gene drive is crossed with the element “A” containing organism strain thereby creating offspring that are complete N-element daisy drive organisms. In another non-limiting example, organisms that include the designed “generic” N−1 daisy chain gene drive may be released into an environment to initiate a daisy chain drive effect that spreads the gene encoding the RNA-guided DNA nuclease (encoded in element “B”) through a local population of the wild-type organism, after which one or more organisms of the same background strain as the N−1 organisms, but that include an element encoding another desired genome effector or modulation effect, for example alteration or suppression, (designated as a “Z” element) can be released into the N−1 daisy chain gene drive organism population to accomplish the desired genome modulation effect. It will be understood that if the desired gene modulation effect is suppression, the release will eliminate the RNA-guided DNA nuclease from the population, and if the desired genome modulation is alteration or gene expression, it can be accomplished two or more times in series or in parallel by releasing into the N−1 organism population, two, three, four, five, six, seven, or more organism strains prepared such that each includes a different Z element.

Certain aspects of the invention include methods of preparing cells, cell lines, and/or organisms that include daisyfield drives and daisy chain gene drives that encode Cas9. In some embodiments of such a daisy chain gene drive system, methods of the invention can be used to design, construct and use one ‘generic’ daisy chain drive strain per organism species. Using such a strategy, one or more “A” elements carrying payloads can be added directly to the generic daisy chain drive strain, wherein each “A” element also encodes guide RNAs sufficient to drive itself in the presence of the expressed Cas9. This non-limiting example of single-strain, single-stage approach can be designed, constructed, and implemented using methods of the invention.

Another method of the invention may include preparing a generic daisyfield drive organism and/or daisy drive organism strain that includes the Cas9 gene, and is released into a target region resulting in the spread of the Cas9 gene through a population of the organism in the target region. One or more additional organism strains can be prepared in the same wild-type organism strain as the generic daisy drive organism strain, but that don't include the N−1 daisy chain gene drive, but that do include one or more different “A” elements each designed to produce an desired effect on a selected target gene. The “A” element strain can also be released into the target region and matings between “N−1” strain organisms and “A” element strain organisms result in offspring that include both the “A” and “N−1” elements, and the presence of the full “N” daisy chain gene drive produces the desired effect on the preselected target gene(s). This non-limiting example of a multi-strain, single-stage approach can be designed, constructed, and implemented.

Another embodiment of the invention includes preparing a generic (N−1) daisy chain drive strain that is released into a region in the wild and the spread of the Cas9 gene in the region can be monitored. The monitoring results identify the exact region that was affected by the release. Optionally, spread within this region may be adjusted by releasing wild-type organisms, thereby shifting the ratio of the N−1 organism strain to the wild-type organism strain. When acceptable release numbers and parameters have been determined, a subsequent release of daisy chain drive strains carrying “A” elements that have been designed to produce a desired effect on a selected target gene, would then initiate the desired effect. Methods of the invention to design, construct and implement daisy chain gene drives and systems, may be used in additional strategies for population control.

### Gene Drive Components

Aspects of the invention include methods of preparing cells, cell lines, and/or organisms that include daisy chain gene drives. Daisy chain gene drives that may be delivered into a cell or organism may be designed and constructed using embodiments of methods of the invention. Design methods of the invention are directed to genome editing systems comprising components that can be separately encoded as nucleic acid sequences that are delivered into the genome a cell or organism. A daisy chain gene drive system and daisyfield drive system that may be designed using methods set forth herein may include one or more of the design, construction, and testing of one or more components of the daisy chain gene drive and daisyfield drive, including, but not limited to: guide RNAs, guided DNA binding proteins, nucleic acid-guided DNA binding proteins, RNA-guided DNA binding proteins, DNA-guided DNA binding proteins, promoter/enhancer/3′UTR sequences, housekeeping gene sequences, promoter sequences, predetermined target genes, tRNA sequences, and sequences encoding detectable labels, such as but not limited to fluorescent labels.

Design methods of the invention may be applied when a gene drive system has been selected and in some embodiments include identification of a target gene in the genome of a host cell or organism into which the gene drive will be delivered. As used herein the term “host” or “target” when used in reference to a cell, cell line or organism, means a cell, cell line, or organism, respectively that includes a daisy chain gene drive and/or daisyfield drive system designed using one or more methods of the invention. In some embodiments of the invention, a host cell is a germline cell.

### Target Genes

Target genes, also referred to herein as target nucleic acids, may include any nucleic acid sequence having an effect that is of interest to be modulated using a daisy chain gene drive and/or daisyfield drive of the invention. In some aspects of the invention a target gene comprises DNA, which may be double-stranded DNA or single-stranded DNA. A gene selected as target gene in a daisyfield and/or daisy chain gene drive may be a nucleic acid sequence in the genome of a host cell. A daisyfield and/or daisy chain gene drive of the invention may, in some aspects of the invention, be designed such that it includes a gene drive cassette comprising one or more of: a promoter/enhancer/3′UTR sequence, a nucleic acid-guided DNA binding protein, an RNA-guided DNA binding protein gene sequence, and one or more RNA guide sequences. When expressed in a host cell the promoter/enhancer/3′UTR may drive expression of the RNA-guided DNA binding protein gene, which, in conjunction with the RNA guide sequences is directed to the selected target gene. One or more design methods of the invention in conjunction with routine methods in the art, can be used to identify and select a target gene, and to design guide RNAs having a sufficient level of activity and specificity to guide and position a DNA binding protein to a nucleic acid sequence adjacent, or in close proximity, to the target gene sequence. In certain daisyfield and/or daisy chain gene drives an expressed DNA binding protein has nuclease activity and when positioned in relation to the target gene, a DNA binding protein cuts the target gene and disrupts the normal effect/action of the target gene in the cell.

Assays described herein, and others known in the art, can be used to determine whether a designed guide RNA and DNA binding protein complex binds to or co-localizes with the host DNA in a manner in that results in a desired effect on the target nucleic acid. For example, though not intended to be limiting, if a desired effect on a target gene is to inhibit or suppress a target gene's expression, assays can be performed to determine whether or not the one more designed guide RNAs and DNA binding proteins, is effective to reduce transcription or expression of the target gene. As a non-limiting example, a transcription activity reporter assay described elsewhere herein may be used to determine whether a designed guide RNA and DNA binding protein have a desired effect on a selected target gene.

In some aspects of the invention a target gene is a haploinsufficient gene, which is a gene for which a single copy is insufficient for normal growth and division of a cell in which it is located. A target gene useful in daisyfield and/or daisy chain gene drives of the invention may also be a recessive gene, and the action or function of altering or disrupting the gene may correspond to: sex-specific infertility, infertility, sex-specific viability, or viability. In some aspects of the invention a target gene is a gene encoding a ribosomal protein. In certain aspects of the invention, a target gene may include nucleic acid sequences present on either side of an intron. In some methods of the invention, art-known haploinsufficient genes may be used to design, construct, and implement a daisyfield and/or daisy chain gene drive system of the invention. In certain aspects of the invention, a review of the scientific literature and/or application of routine genetic testing techniques can assist in identifying suitable candidate target genes. Methods are provided herein and are known in the art that can be used to identify and test candidate target genes for use in designing, constructing, and implementing daisyfield and/or daisy chain gene drives of the invention.

It will be understood that selecting a target gene for inclusion in a daisyfield and/or daisy chain gene drive of the invention may be based, at least in part, on the role of the target gene in the daisyfield and/or daisy chain gene drive. For example, in certain aspects of daisyfield and/or daisy chain gene drives of the invention, a target gene may be selected for a “drive element”, non-limiting examples of which are: a non-“A” element, an “A” element carrying a cargo gene, and an “A” element that coordinates drive of a number of different changes that result from the daisy chain gene drive system. For such drive elements, non-limiting examples of suitable genes for selection are haploinsufficient genes and genes that are important for fitness of the host cell or organism. In some aspects of daisyfield and/or daisy chain gene drives of the invention, a target gene may be selected for a “payload element”, non-limiting examples of which include: an “A” element and a gene that is one of a set of genes altered by simultaneous changes that result from the daisyfield and/or daisy chain gene drive system. For such payload elements, non-limiting examples of suitable genes for selection are: any gene, but which may be a gene that is important for fitness of the host cell or organism, a gene to be suppressed, and a gene that is important in fertility and/or viability of the host cell or organism, as described elsewhere herein.

In some aspects of the invention, a target gene is a large ribosomal subunit gene and in certain aspects of the invention, a target gene is a small ribosomal subunit gene. For example, though not intended to be limiting: a target gene may be one of: RpL1, RpL2, RpL3, RpL4, RpL5, RpL6, RpL7, RpL8, RpL9, RpL10, RpL11, RpL12, RpL13, RpL14, RpL15, RpL16, RpL17, RpL18, RpL19, and. RpL20. Additional art-known large ribosomal subunit genes and variants thereof are suitable as target genes in methods of the invention. Other non-limiting examples of target genes are: RpS1, RpS2, RpS3, RpS4, RpS5, RpS6, RpS7, RpS8, RpS9, RpS10, RpS11, RpS12, RpS13, RpS14, RpS15, RpS16, RpS17, RpS18, RpS19, and. RpS20. Additional art-known small ribosomal subunit genes and variants thereof, large ribosomal subunit genes and variants thereof, and other genes and variants thereof, are suitable as target genes in methods of the invention.

### DNA Binding Proteins

Components of a daisyfield and/or daisy chain gene drive system designed using at least one method of the invention may include DNA binding proteins and functional variants thereof. In certain aspects of the invention, a DNA-binding protein may be a nucleic acid-guided DNA binding protein. Non-limiting examples of types of nucleic acid DNA-binding proteins that may be used in some embodiments of daisyfield and/or daisy chain gene drives of the invention include: RNA-guided DNA-binding proteins and DNA-guided DNA-binding proteins. DNA binding proteins are known in the art, and include, but are not limited to: naturally occurring DNA binding proteins, a non-limiting example of which is a Cas9 protein, which has nuclease activity and cuts double stranded DNA. Cas9 proteins and Type II CRISPR systems are well documented in the art. (See for example, Makarova et al., Nature Reviews, Microbiology, Vol. 9, June 2011, pp. 467-477, the content of which is incorporated by reference herein in its entirety.) As used herein, the term “DNA binding protein having nuclease activity” refers to DNA binding proteins having nuclease activity and also functional variants thereof. SEQ ID NO: 1 is an amino acid sequence of Cas9, and may be used in methods of the invention as an RNA-guided DNA binding protein having nuclease activity. Functional variants of SEQ ID NO: 1 can also be used in daisyfield and/or daisy chain gene drives designed, constructed, and/or implemented using one or more methods of the invention. A functional variant of SEQ ID NO: 1 differs in amino acid sequence from SEQ ID NO: 1, referred to as the variant's “parent” sequence, while retaining from a least a portion to all of the nuclease activity of its parent protein.

In some embodiments, a daisyfield and/or daisy chain gene drive of the invention may include a DNA-guided DNA-binding nuclease. Information on identification and use of DNA-guided binding proteins, for example in DNA-guided genome editing systems, is available in the art (Gao, F., et al., Nature Biotech online publication, May 2, 2016: doi:10.1038/nbt.3547, the content of which is incorporated herein by reference in its entirety).

A DNA binding protein having nuclease activity function to cut double stranded DNA that may be used in aspects of methods of the invention can include DNA binding proteins that have one or more polypeptide sequences exhibiting nuclease activity. A DNA binding protein with multiple regions that have nuclease activity may comprise two separate nuclease domains, each of which functions to cut a particular strand of a double-stranded DNA. Polypeptide sequences that have nuclease activity are known in the art, and non-limiting examples include: a McrA-HNH nuclease related domain and a RuvC-like nuclease domain, or functional variants thereof. In S. pyogenes, a Cas9 DNA binding protein creates a blunt-ended double-stranded break that is mediated by two catalytic domains in the Cas9 binding protein: an HNH domain that cleaves the complementary strand of the DNA and a RuvC-like domain that cleaves the non-complementary strand. [See Jinke et al., Science 337, 816-821 (2012), the content of which is incorporated by reference herein in its entirety]. Cas9 proteins are known to exist in many Type II CRISPR systems, see for example, Makarova et al., Nature Reviews, Microbiology, Vol. 9, June 2011, pp. 467-477, supplemental information, the content of which is incorporated herein by reference in its entirety. The Cas9 protein may be referred by one of skill in the art in the literature as Csn1. Alternatives to Cas9 include but are not limited to Cpf1 proteins from Type V CRISPR systems. In certain aspects of the invention, a daisyfield and/or daisy chain gene drive may include a DNA binding protein that does not have nuclease activity.

### Guide Nucleic Acids

Methods of the invention, in part, include design, construction, and implementation of daisyfield and/or daisy chain gene drives that include guide nucleic acid molecules, non-limiting examples of which are guide RNAs and guide DNAs. Information relating to guide DNAs can be found in Gao, F., et al., Nature Biotech online publication, May 2, 2016: doi:10.1038/nbt.3547, the content of which is incorporated herein by reference in its entirety. Guide RNAs are also referred to herein as short guide RNAs, sgRNAs, and gRNAs. A guide RNA is designed and selected such that it is complementary to a DNA sequence of the selected target gene in the genome of a cell, and so the guide RNA acts in complex with a DNA binding protein, or variant thereof to direct degradation of the complementary sequence within the target gene.

In some aspects of the invention methods are provided that can be used to prepare a daisyfield and/or daisy chain gene drive in which an exogenous nucleic acid sequence is delivered into a host cell, and is expressed in the cell to produce a nucleic acid-guided DNA binding protein having nuclease activity, and one or more guide nucleic acids. In a non-limiting example: a vector comprising a sequence encoding the one or more guide RNAs and the RNA-guided DNA binding protein may be designed and used in daisyfield and/or daisy chain gene drives of the invention. Expression of the vector sequences in the host cell results in production of a complex of the RNA-guided DNA binding protein and guide RNAs that is directed by the guide RNA(s) to the preselected target gene, where the complex co-localizes to, or bind with, the target gene and the target gene is cleaved in a site-specific manner by the nuclease activity of the RNA guided DNA binding protein.

Useful methods of designing guide RNAs to direct an RNA-guided DNA binding protein to a selected target gene are provided herein and are also known in the art. Guide RNAs can be designed, prepared, tested, and selected for use in a daisyfield and/or daisy chain gene drive system of the invention using one or more of the methods provided, in conjunction with knowledge in the art relating to DNA binding, vector preparation and use, RNA-guided DNA binding proteins, CRISPR system components and implementation, etc. The length of a guide RNA used in a daisyfield and/or daisy chain system of the invention may be at least 30, 40, 50, 60, 70, 80, 90, 100, 110, 120, 130, 140, 150, 200, 250, 300, 350, 400, 450, and 500 base pairs, including all integers between those listed. It will be understood that a maximum or minimum permissible length of a guide RNA is limited to a length at which the guide RNA functions as a guide RNA in a daisyfield and/or daisy chain gene drive of the invention.

Art-known methods can be used to design, construct, and implement a plurality of diverse/divergent guide RNA for an RNA-guided DNA nuclease. Methods such as those set forth herein and those set forth in International Application No. PCT/US17/31777 can be used to prepare divergent guide RNAs and can be used to determine activity of the divergent guide nucleic acids. Methods of the invention may include use of repetitive sequences. As used herein, the term “elements” when used in the context of preparing divergent guide RNA sequences means the backbone sequence of the guide RNA that is recognized by the nuclease and is capable of directing the nuclease to cut a predetermined target sequence.

Non-limiting examples of guide RNAs that may be useful in methods of the invention are set forth herein as SEQ ID NO: 3-34. The length of a guide RNA for use in methods of the invention may be at least 30, 40, 50, 60, 70, 80, 90, 100, 110, 120, 130, 140, 150, 200, 250, 300, 350, 400, 450, and 500 base pairs, including all integers between those listed. It will be understood that a maximum or minimum permissible length of a guide RNA is limited to a length at which the guide RNA functions as a guide RNA in a daisy chain gene drive of the invention.

### Divergent RNA Sequences

In certain embodiments of methods of the invention such as, but not limited to: under dominance and daisy field methods, can be prepared using readily synthesized double-stranded (ds) DNA sequences to produce multiple guide RNAs. The produced multiple (or plurality of) guide RNAs can prepared such that they are able to direct a CRISPR-type protein (complex) to multiple target sites within a cell. Art-known methods and methods disclosure herein can be used to prepare divergent guide RNA sequences and the use of divergent guide RNA sequences results in the ability to target a number of targets sites within the same cell. Divergent sequences may be prepared using methods disclosed herein and/or art-known methods and used in embodiments of daisy chain gene drives and daisyfield gene drives as disclosed herein, and also for other uses in cells and organisms. For example, divergent guide RNA sequences can be used to prepare a plurality of sequences that have minimal sequence homology/identity between themselves and so can be used for multi-targeting. As used herein, the term “multi-targeting” when used in the context of a plurality of divergent sequences means that the sequences are designed such that they target multiple different sequence sites, for example in a cell in which they are expressed.

It has previously been prohibitively difficult to synthesize repetitive sequences. Methods disclosure herein may be used to obviate this difficulty and permit rapid preparation of DNA sequences capable of expressing multiple guide RNAs. In some aspects of the invention, available information on sequences of interest is used to create a map or diagram of guide RNA that shows each possible individually accepted change throughout the structure of the guide RNA. After acceptable sequence changes have been mapped and identified, several 5, 10, 15, 20, 25, 30, 35, 40, 45, or more elements are designed that combine different combinations of the of these accepted changes. The elements are designed to minimize the length of sequences that are shared between the designed elements. Thus, the elements are designed to minimize the length of any sequences common to two or more of the designed elements. As used herein, the term “element” when used in the context of preparing divergent nucleic acid sequences, such as divergent guide RNA sequences, means the backbone sequence of the guide RNA that is recognized by a preselected nuclease and that is capable of directing the nuclease to cut a preselected target sequence.

The activity and functionality of designed backbones of the guide RNA sequences are determined and those that have high activity can be selected. The activity of the designed divergent sequences can be tested using transcription assays such as those disclosed herein, or using other art-known assays. The activity of the guide RNA is also referred to herein as “function” of the guide RNA. Thus, a guide RNA that has a high activity is one that functions in a desired manner, for example: to be recognized by a nuclease and directing the nuclease to a preselected target gene sequence. Identified high-activity guide RNAs can be used in methods of the invention to construct evolutionarily stable homing-based gene drive systems that target multiple sites to overcome the evolution of mutations that block cutting. Divergent guide RNAs prepared using methods of the invention significantly reduce the chance of recombination between homologous sequences within the drive cassette, which is a major problem for highly repetitive drive cassettes (Simoni et al Nucl. Acids Res. 2014 http://dx.doi.org/10.1093/nar/gku387), the resulting drive system will be stable.

An example of the method of preparing divergent sequences, includes, but is not limited to: identifying divergent guide RNAs with high activity using methods described above and also in Method 1.0 and expressing multiple guide RNAs from a single promoter using tRNA processing [see Xie et al. (2015) PNAS doi:10.1073/pnas.1420294112, Port and Bullock (2016) bioRxiv doi:10.1101/046417, the content of each of which is incorporated herein in its entirety]. The guide RNA sequences and tRNA sequences can be synthesized along with a promoter that has been identified to work well in a target organism in which the guide RNAs will be implemented. A non-limiting example of a promoter that may be included is a U6 promoter or equivalent. Non-limiting examples of a sequence of a promoter, tRNAs, and a plurality of divergent guide RNAs are: U6promoter-tRNA1-sgRNA1-tRNA2-sgRNA2-tRNA3-sgRNA3-tRNA4-sgRNA4; promoter-tRNA1-sgRNA1-tRNA2-sgRNA2-tRNA3-sgRNA3-tRNA(N)-sgRNA(N), wherein “N” is the highest number in the series, for example, if there are four tRNAs and four sgRNAs, the series would be: promoter-tRNA1-sgRNA1-tRNA2-sgRNA2-tRNA3-sgRNA3-tRNA4-sgRNA4, if there are six tRNAs and six sgRNAs the series would be: promoter-tRNA1-sgRNA1-tRNA2-sgRNA2-tRNA3-sgRNA3-tRNA6-sgRNA6. “N” may be independently determined for sgRNAs and tRNAs. “N” may be 2, 3, 4, 5, 6, 7, 8, 9, 10, 11, 12, 13, 14, 15, 16, 17, 18, 19, 20, 25, 30 or more. Synthesis of the designed DNA can be done using art-known methods such as, but not limited to: Integrated DNA Technologies gBlocks (Integrated DNA Techologies, Coralville, Iowa) and ThermoFisher GeneArt Strings (Thermo Fisher Scientific).

Methods for preparing a plurality of divergent nucleic acid sequences as set forth herein in reference to preparing divergent sequences for daisy chain gene drives, can also be used to prepare divergent sequences for use in other multiplexing methods, including but not limited to gene drive methods. The resulting sequences can be used to target multiple target sequences.

### Additional Components

Additional components used in a daisy chain gene drive and/or a daisy field drive of the invention include, but are not limited to: components included in a vector delivered to a cell as part of a daisy field and/or daisy chain gene drive of the invention. Sequences such as: promoter sequences, enhancer sequences, 3′ untranslated region (3′UTR) sequences can be included. Those skilled in the art will understand how to use such sequences to design, construct, and implement daisy chain gene drives of the invention based on methods, components, and strategies disclosed herein and art-known gene drive methods and components [see for example: International Application No. PCT/US17/31777; Noble C, et al., (2016) bioRxiv (preprint), doi: dx.doi.org/10.1101/057307; Min J, et al., (2017) bioRxiv (preprint) doi: dx.doi.org/10.1101/104877; and Min J, et al. (2017) bioRxiv (preprint), doi: dx.doi.org/10.1101/115618, the content of which is incorporated by reference herein in its entirety).

### Variants

Components of a daisy chain gene drive may include sequences described herein, or designed using one or more methods of the invention and may also include functional variants of such sequences. A variant polypeptide may include deletions, point mutations, truncations, amino acid substitutions and/or additions of amino acids or non-amino acid moieties, as compared to its parent polypeptide. Modifications of a polypeptide of the invention may be made by modification of the nucleic acid sequence that encodes the polypeptide. The terms “protein” and “polypeptide” are used interchangeably herein as are the terms “polynucleotide” and “nucleic acid” sequence. A nucleic acid sequence may comprise genetic material including, but not limited to: RNA, DNA, mRNA, cDNA, etc. As used herein with respect to polypeptides, proteins, or fragments thereof, and polynucleotides that encode such polypeptides the term “exogenous” means the one that has been introduced into a cell, cell line, organism, or organism strain and not naturally present in the wild-type background of the cell or organism strain.

In certain embodiments of the invention, a polypeptide or nucleic acid variant may be a polypeptide or nucleic acid, respectively that is modified from its “parent” polypeptide or nucleic acid sequence. Variant polypeptides and nucleic acids can be tested for one or more activities (e.g., delivery to a target gene, suppression of a target gene, etc.) to determine which variants are possess desired functionality for use in a daisy chain gene drive of the invention.

The skilled artisan will also realize that conservative amino acid substitutions may be made in a polypeptide, for example in a Cas9 polypeptide, to design and construct a functional variant useful in a daisy chain gene drive of the invention. As used herein the term “functional variant” used in relation to polypeptides is a variant that retains a functional capability of the parent polypeptide. As used herein, a “conservative amino acid substitution” refers to an amino acid substitution that does not alter the relative charge or size characteristics of the polypeptide in which the amino acid substitution is made. Conservative substitutions of amino acids may, in some embodiments of the invention, include substitutions made amongst amino acids within the following groups: (a) M, I, L, V; (b) F, Y, W; (c) K, R, H; (d) A, G; (e) S, T; (f) Q, N; and (g) E, D. Polypeptide variants can be prepared according to methods for altering polypeptide sequence and known to one of ordinary skill in the art such. Non-limiting examples of functional variants of polypeptides for use daisy chain gene drives of the invention are functional variants of a Cas9 polypeptide, functional variants of detectable label sequences, etc.

As used herein the term “variant” in reference to a polynucleotide or polypeptide sequence refers to a change of 1, 2, 3, 4, 5, 6, 7, 8, 9, 10, or more nucleic acids or amino acids, respectively, in the sequence as compared to the corresponding parent sequence. For example, though not intended to be limiting, a variant guide RNA sequence may be identical to that of its parent guide RNA sequence except that 1, 2, 3, 4, 5, 6, 7, 8, 9, 10, or more nucleic acid substitutions, deletions, insertions, or combinations thereof, and thus is a variant of the parent guide RNA. In another non-limiting example, the amino acid sequence of a variant Cas9 nuclease polypeptide may be identical to that of its parent Cas9 nuclease except that it has 1, 2, 3, 4, 5, 6, 7, 8, 9, 10, or more amino acid substitutions, deletions, insertions, or combinations thereof, and thus is a variant of the parent Cas9 nuclease. Certain methods of the invention for designing and constructing daisy chain gene drives include methods to prepare functional variants of daisy chain gene drive components such as guide nucleic acids, guide RNAs, and guide DNAs. Methods provided herein, and other art-known methods can be used to prepare candidate guide sequences that can be tested for function and to determine whether they retain sufficient activity for use in a daisy chain gene drive of the invention.

Methods of the invention provide means to test for activity and function of variant sequences and to determine whether a variant is a functional variant and is suitable for inclusion in a daisy chain gene drive of the invention. Suitability can, in some aspects of methods of the invention, be based on one or more characteristics such as: expression; cell localization; gene-cutting activity, efficacy in modulating activity of a target gene, etc. Functional variant polypeptides and functional variant polynucleotides that may be used in daisy chain gene drives of the invention may be amino acid and nucleic acid sequences that have at least 75%, 80%, 85%, 90%, 95%, 96%, 97%, 98%, or 99% identity to their parent amino acid or nucleic acid sequence, respectively.

Art-known methods can be used to assess relative sequence identity between two amino acid or nucleic acid sequences. For example, two sequences may be aligned for optimal comparison purposes, and the amino acid residues or nucleic acids at corresponding positions can be compared. When a position in one sequence is occupied by the same amino acid residue, or nucleic acid as the corresponding position in the other sequence, then the molecules have identity/similarity at that position. The percent identity or percent similarity between the two sequences is a function of the number of identical positions shared by the sequences (i.e., % identity or % similarity=number of identical positions/total number of positions×100). Such an alignment can be performed using any one of a number of well-known computer algorithms designed and used in the art for such a purpose. It will be understood that a variant polypeptide or polynucleotide sequence may be shorter or longer than their parent polypeptide and polynucleotide sequence, respectively. The term “identity” as used herein in reference to comparisons between sequences may also be referred to as “homology”.

### Preparation and Delivery

Components of daisy chain gene drives of the invention may be delivered into a cell using standard molecular biology techniques. In certain aspects of the invention, vectors are used to implement a daisy chain gene drive of the invention, for example, to deliver a daisy chain gene drive element to a cell. As used herein, the term “vector” used in reference to delivery of components of a daisy chain gene drive system refers to a polynucleotide molecule capable of transporting between different genetic environments another nucleic acid to which it has been operatively linked. One type of vector is an episome, i.e., a nucleic acid molecule capable of extra-chromosomal replication. Some useful vectors are those capable of autonomous replication and/or expression of nucleic acids to which they are linked. Vectors capable of directing the expression of genes to which they are operatively linked may be referred to herein as “expression vectors”. Other useful vectors, include, but are not limited to viruses such as lentiviruses, retroviruses, adenoviruses, and phages. Vectors useful in some methods of the invention can genetically insert one or more of a gene drive cassette into a dividing or a non-dividing cell and can insert one or more daisy chain gene drive elements into an in vivo or in vitro cell.

Vectors useful in methods of the invention may include sequences including, but not limited to one or more promoter sequences, enhancer sequences, 3′ untranslated region (3′UTR) sequences, guide nucleic acid sequences, guide RNA sequences, DNA binding protein encoding sequences, detectable label encoding sequences, etc. Methods of the invention can be used to design and construct vectors comprising components of daisy chain gene drive systems. Expression vectors and methods of their use are well known in the art.

Promoters that may be used in methods and vectors of the invention include, but are not limited to, cell-specific promoters or general promoters. Methods for selecting and using cell-specific promoters and general promoters are well known in the art.

### Hosts, Cells, Cell Lines, and Organisms

One or more methods of the invention for designing and constructing daisy chain gene drives as described here can be applied to prepare and deliver a daisy chain gene drive into a host cell or organism. A host cell or organism is one to which a daisy chain gene drive is delivered. In some aspects of the invention, a host cell and its progeny are understood to be member of a cell strain that includes the daisy chain gene drive, and may be referred to as daisy gene drive strain or a daisy drive strain. Similarly, a host organism and its progeny that include a daisy chain gene drive designed or prepared using one or more methods of the invention, may be referred to as an organism of a daisy drive strain, or daisy chain gene drive strain organisms, or simply as a daisy drive strain. A mutant lineage of an organism that is prepared using a daisy chain gene drive may be also be referred to as a “strain”.

Daisy chain gene drive systems may be delivered to cells and organisms at various developmental stages of the cells and organisms, respectively. Non-limiting examples of stages of cells to which a daisy chain gene drive system of the invention may be delivered or included are: embryonic cells, germline cells, gametes, cells that can give rise to a gamete, zygotes, pre-meiotic cells, post-meiotic cells, fully-differentiated cells, and mature cells. Cells at this stages may be isolated cells, cells in cell lines, cells in cell, tissue, or organ culture, cells that are within an organism. In certain embodiments of the invention, a cell is a zygote, a gamete, a cell that is able to give rise to a gamete, a germline cell, etc.

Daisy chain gene drive systems designed and constructed using one or more methods of the invention may be delivered to and included in cells of various organisms. In some aspects of the invention, a cell or organism is a vertebrate or an invertebrate cell or organism. In certain aspects of the invention, a cell or organism is a eukaryotic or prokaryotic cell or organism. Non-limiting examples of organisms to which a daisy chain gene drive designed using one or more methods of the invention may be delivered to or included in are: insects, fish, reptiles, amphibians, mammals, birds, protozoa, annelids, mollusks, echinoderms, flatworms, coelenterates, and arthropods, including arachnids, crustaceans, insects, and myriapods. In some aspects of the invention an organism selected for inclusion of a daisy chain gene drive designed and constructed is an organism selected because of a population of the organism that is of interest to control or modify. As a non-limiting example, if it is of interest to control a wild population of a species of mosquito in an area or region, one or more methods of the invention are used to design and construct a daisy chain gene drive for that specific species; the designed daisy drive gene system is delivered to and included in one or more host mosquitoes of that species; one or more of the daisy chain gene mosquito strain is released into the population of wild mosquitoes; and the release of the daisy chain gene drive mosquito strain organisms controls and modulates the wild mosquito population.

In certain aspects of the invention, an organism species to which a daisy chain gene drive designed using one or more methods of the invention may be delivered to, or included in, is a species that serves as a vector for disease affecting humans, animals, or plants. The term “vector” as used herein in reference to disease transfer, means any organism that carries and transmits an infectious pathogen into another living organism.

Some embodiments of methods of the invention for one more of designing, constructing, implementing daisy chain gene drives and systems may also be used to design, prepare, and deliver daisy chain gene drives to plants and cells thereof, including: monocots and dicots, weeds, invasive plants, poisonous plants, aquatic plants, terrestrial plants, recombinant plants, etc.

Combination with Traditional Population Control Measures

It will be understood that daisy chain gene drives designed and/or constructed using one or more methods of the invention, can be introduced into cells, cell lines, and/or organisms that are released into wild populations of organisms of the same background strain. Such releases may be used in methods to suppress a wild population of the organisms. Population reduction using daisy chain gene drives designed using methods of the invention may be used in combination with other art-known means to reduce or control the size, range, density, etc. of a population of organisms.

A population of organisms may be a local population, non-limiting examples of which is a population in a geographically defined region, such as a forest, swamp, field, pond, island, etc. and a population in a politically defined region, such as a town, state, county, etc. For example, though not intended to be limiting: to reduce the size of a wild population of a species of mosquitoes that is a known vector for a pathogen, such as malaria, eastern equine encephalitis (EEE), etc., one more methods of the invention can be used to design, construct and/or implement a daisy chain gene drive system that when included in organisms released into the wild population, is effective to decrease the size of the mosquito population.

It will be understood that daisy chain gene drive systems of the invention can be used alone or used in any combination of: before, simultaneously with, and after use of one or more alternative methods to modulate a wild population. Non-limiting examples of alternative modulation methods include: administration of pesticides, herbicides, anti-fertility agents; habitat eradication or disruption; release of organisms predatory upon the wild population; etc. Those skilled in the art will be able to identify additional population control means and to use alternative population modulation methods in combination with daisy chain gene drive methods of the invention.

### Population Modulation/Control

Aspects of the invention are drawn to methods to design, construct, and deliver daisy chain gene drives into cells and organisms and the release of such organisms into wild populations to modulate and control populations of species. For example, though not intended to be limiting, includes a daisy chain gene drive designed, constructed, and/or prepared using one or more methods of the invention that is released into a wild population of an invasive species to control or eliminate that population of the invasive species. Daisy chain gene drives described herein have particular practical utility with vector-borne diseases. Malaria, dengue, yellow fever, trypanosomiasis, leishmaniasis, Chagas disease, and Lyme disease are non-limiting examples of disease caused by pathogens that are spread using vectors. Risk to subjects from infection or illness-promoting organisms may be reduced or eliminated by reducing a wild population of the organism or a vector thereof, using one or more daisy chain gene drives designed using methods of the invention. Subjects that may be protected using daisy chain gene drives designed using one more methods of the invention include, but are not limited to: humans, domesticated animals, agricultural animals, agricultural plants, wild animals, native/wild plant etc.

### Field Trials and Safeguards

Certain aspects of methods of the invention include field testing. Unlike previous global gene drive systems, methods of the invention provide designs for daisy chain gene drives that can be safely tested in field trials. Daisy drive systems, designed using methods of the invention, may be capable of mimicking the molecular effects of any given global drive on a local level, and may be powerful enough to eliminate all copies of an unwanted global drive system through local immunizing reversal or population suppression, and may be field tested. Daisy drive systems designed and constructed using methods of the invention, may provide controlled and persistent population suppression by linking a sex-specific effect to a genetic locus unique to the other sex. For example, though not intended to be limiting, female fertility genes such as those recently identified in malarial mosquitoes (Hammond, A. et al., Nat Biotechnol. 2015 Dec. 7; doi:10.1038/nbt.3439) can be targeted by a genetic load daisy drive whose basal element is located on the Y chromosome or an equivalent male-specific locus (FIG. 16). These daisy chain gene drive males would suffer no fitness cost due to suppression relative competing wild-type males. Another non-limiting example is a 3-element daisy drive system wherein female fertility gene disruption occurs early in development, creating a male-linked dominant sterile-daughter effect that is otherwise very difficult to generate genetically. Methods of designing and constructing daisy chain gene drives as set forth herein can be used to titrate local population levels of an organism in a controlled and reversible manner, and may be useful in activity such as modulating populations of organisms, reducing populations of detrimental organisms, and studying organisms and their ecological interactions.

### Evolutionary Stability and CRISPR Multiplexing

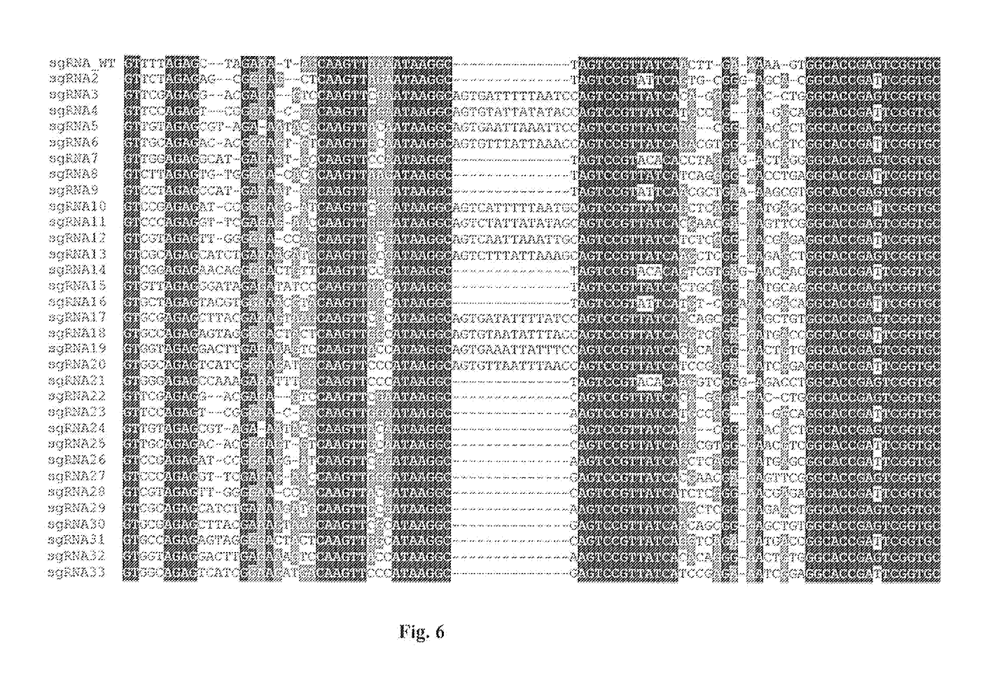

Aspects of the invention include design and construction methods that overcome previous technological limitations and permit safe use of daisy drive elements. Specifically, design and construction methods of the invention can be used to reduce or eliminate risk of a recombination event that would move one or more guide RNAs within basal element of the chain into a higher element. Such a recombination event would convert a linear daisy drive chain into a self-sustaining CRISPR gene drive ‘necklace’ (FIG. 5). Methods of the invention include design strategies that eliminate regions of homology between the elements. Aspects of methods of the invention include, removal of promoter homology, for example, by using different U6, H1, or tRNA promoters for each element. Various promoters are known in the art and may be used in methods of the invention, see for example, Port et al. (2014) PNAS doi:10.1073/pnas.1405500111; Ranganathan et al (2014) Nat. Comm. doi:10.1038/ncomms5516; and Mefferd et al (2015) RNA doi:10.1261/rna.051631.115, the content of each of which is incorporated herein by reference. Methods of the invention, in some aspects include design and construction of daisy chain gene drives that include multiple guide RNAs expressed from a single promoter using tRNA processing (see for example: Xie et al. (2015) PNAS doi:10.1073/pnas.1420294112, Port and Bullock (2016) bioRxiv 10.1101/046417), the content of each of which is incorporated herein by reference) or by connecting a pair of sgRNAs by a short linker. In certain aspects of designs of the invention, each gene drive element includes guide RNAs that are greater than 80 base pairs in length.

### Design, Construction, and Use

Methods to design and construct daisyfield gene drives have been developed. Use of the methods singly, in combination of two or more, and in combination of one or more with other design methods for gene drives permits daisyfield gene drives to be designed, constructed, and used. Daisyfield gene drives prepared using one or more methods described herein are included in cells, cell lines, and/or organisms.

Daisyfield gene drives designed using methods provided herein can be used to address otherwise intractable ecological problems, with a level of safety inherent in their design, that reduces or eliminates a likelihood of global effects as occurs for conventional gene drive organisms that are released into the wild. Daisyfield drive elements and systems designed and/or constructed using methods provided herein are used to reduce instances and control vector-borne and parasitic diseases such as, but not limited to: malaria, schistosomiasis, dengue, and Zika virus. They may also be used to control or eliminate populations of agricultural pests or invasive species.

Daisyfield drive elements and systems designed and/or constructed using one or more methods provided herein, include molecular constraints that when included in an organism or population of organisms, limit geographic spread in a tunable manner. Daisyfield drive design and construction methods set forth herein are used in ecological engineering by enabling local communities to make decisions concerning their own environments.

### Multiplex CRISPR Methods, Preparations, and Use

Aspects of the invention, in part, include multiplexing methods that direct one or more CRISPR proteins to do one or more of binding and cutting a plurality of target DNA sequences. In some aspects, CRISPR multiplexing comprises interspersing different types of guide RNAs in a repetitive array. Some embodiments of multiplexing methods comprise inserting a preselected DNA sequence into a plurality of repeated regions in the genome of an organism. This method can be used to prepare an engineered organism strain. In some aspects of the invention, the insertion of the preselected DNA sequence into a plurality of repeated regions in the genome is done in a plurality of organisms of the strain, which generates a plurality of the engineered organisms. Such engineered organism can be released into wild populations comprising non-engineered organisms of the original strain.

Various methods can be used to insert the preselected DNA sequence (for example, though not intended to be limiting, a DNA sequences encoding CRISPR polypeptides) into a cell. In some aspects of the invention, a gene cassette comprising the DNA sequence comprising sequences encoding one or more guide RNAs is delivered into one or a plurality of cells. In certain embodiments of the invention, the gene cassette is inserted into a plurality of repeated regions in the genome of the organism and when the one or more encoded guide RNAs are expressed in the cell in the presence of an RNA-guided protein nuclease in the cell, the expressed guide RNAs direct cutting of target DNA sequences on chromosomes of the organism.

In some aspects of the invention CRISPR multiplex methods may include delivery into a cell, a DNA cassette carrying two or more genes that encode CRISPR nucleases. In some embodiments, when expressed, one of the CRISPR polypeptides is capable of processing its own CRISPR RNA (crRNA) array. A non-limiting example of a CRISPR polypeptide capable of processing its own CRISPR RNA (crRNA) array is a Cpf1 polypeptide. In embodiments of the invention, the DNA cassette also includes sequences that flank the encoded CRISPR polypeptides and the presence of the flanking sequences results in expression in the target cell, cell type, or organism. Thus, in some embodiments, a DNA cassette encodes a promoter sequence upstream of an array of guide RNAs corresponding to a CRISPR nuclease, and are positioned such that processing of the crRNAs by their corresponding nuclease results in each guide RNA being liberated from the guide RNAs in such a way as to enable them to bind their appropriate nuclease to form an active CRISPR complex. Multiplexing methods of the invention can be used to activate genes, repress genes, in gene drives. In certain aspects, multiplexing methods are used for virus defense.

Certain compositions of the invention comprise multiplex CRISPR components. Examples of multiplex CRISPR components comprise: DNA cassettes. In some aspects of the invention, a multiplex CRISPR cassette comprises two or more genes that each encodes a an independently selected CRISPR nuclease, and when expressed, one of the CRISPR polypeptides processes its associated CRISPR RNA (crRNA) array. In some embodiments, a multiplex CRISPR DNA cassette also comprises one or more sequences that each affects expression of at least one of the cassette's two or more genes. In some embodiments the affecting expression means it is responsible for expression occurring. An example, though not intended to be limiting, of an affecting sequence is a promoter sequence.

In certain aspects of the invention, each of the DNA cassettes in a multiplex CRISPR composition also comprises sequences encoding: (i) an independently selected promoter sequence and (ii) an array of guide RNAs that correspond to each of the two or more nucleases, wherein the encoded promoter sequences are positioned in the DNA cassettes upstream of the encoded guide RNAs array. In addition, the guide RNAs present are arranged in an array such that processing of a CRISPR RNA (crRNA) by its corresponding nuclease results in each guide RNA being liberated from the others in the array. Once liberated, each liberated guide RNA can bind its appropriate nuclease thereby forming an active CRISPR complex.

Certain embodiments of multiplexing methods of the invention include arrays of guide RNAs that alternate Cas9 sgRNAs with Cpf1 crRNAs. Because Cpf1 does its own processing (cuts at either side of its crRNAs), it will turn the sgRNA-crRNA-sgRNA-crRNA-sgRNA-crRNA-sgRNA chain into individual sgRNA and crRNA fragments that can be bound by Cas9 and Cpf1. Certain embodiments of multiplex compositions of the invention comprise arrays of Cas9 sgRNAs alternating with Cpf1 crRNAs.

Certain aspects of the invention include use of multiplex CRISPR compositions and methods in cells, organism, daisyfield gene drive systems, daisy chain gene drive systems, etc.

### Embodiments of Daisy Field and Daisy Chain Gene Drives

Methods to design and construct RNA-guided gene drives based on CRISPR/Cas9 can be used to prepare daisy field and/or daisy chain gene drive systems of the invention. Use of the methods singly, in combination of two or more, and in combination of one or more with other design methods for gene drives permits daisy chain gene drives to be designed, constructed, and used. Daisy chain gene drives prepared using one or more methods described herein, and/or using one of art-known methods (see for example: International Application No. PCT/US17/31777) are included in cells, cell lines, and/or organisms.

Gene drive elements and systems designed and/or constructed using one or more methods provided herein, include molecular constraints that when included in an organism or population of organisms, limit geographic spread in a tunable manner. Gene drive design and construction methods set forth herein are used in ecological engineering by enabling local communities to make decisions concerning their own environments.

Daisy chain gene drives designed using methods provided herein can be used to address otherwise intractable ecological problems, with a level of safety inherent in their design, that reduces or eliminates a likelihood of global effects as occurs for conventional gene drive organisms that are released into the wild. Daisy gene drive elements and systems designed and/or constructed using methods provided herein are used to reduce instances and control vector-borne and parasitic diseases such as, but not limited to: malaria, schistosomiasis, dengue, and Zika virus. They may also be used to control or eliminate populations of agricultural pests or invasive species.

Gene drive elements and systems designed and/or constructed using one or more methods provided herein, include molecular constraints that when included in an organism or population of organisms, limit geographic spread in a tunable manner. Gene drive design and construction methods set forth herein are used in ecological engineering by enabling local communities to make decisions concerning their own environments.

Designing and Constructing RNA-Guided DNA Nuclease Gene Drive Elements that Target Multiple Sequences but do not Themselves Encode Repetitive Elements.

Methods are provided that, in some embodiments, include targeting multiple sites by identifying sets of guide RNAs with very little homology to one another. Additionally, a set of highly active guide RNA sequences is disclosed in FIG. 6 that have been verified to function with the most commonly used CRISPR system, that of S. pyogenes. These can be encoded in RNA-guided CRISPR gene drive systems to promote high penetrance and evolutionary stability. Guide RNAs may be expressed using a single Polymerase III or (less efficiently) Polymerase II promoter along with sequences promoting processing, such as tRNAs, using previously described methods known to those in the art that are incorporated herein by reference (Xie et al 2015 PNAS doi:10.1073/pnas.1420294112, Mefferd 2015 RNA doi:10.1261/rna.051631.115, Port and Bullock bioRxiv doi:10.1101/046417). Alternatively, two may be expressed from a single Polymerase III promoter using 5-50 base pair linkages between the two guide RNAs. Alternatively, each guide RNA may be expressed from its own promoter, which may be a Polymerase III promoter. Suitable Polymerase III promoters with minimal homology are known to those in the art, e.g. U6, H1, and tRNA promoters (Port et al 2014 PNAS doi:10.1073/pnas.1405500111, Ranganathan et al 2015 doi:10.1038/ncomms5516).

Methods of the invention are provided that, in some embodiments, include targeting multiple sites by identifying sets of guide RNAs with very little homology to one another. Additionally, a set of highly active guide RNA sequences is disclosed in FIG. 6 that have been verified to function with the most commonly used CRISPR system, that of S. pyogenes. A smaller set of active guide RNA sequences is disclosed in Table 1 that have been verified to function with the AsCpf1 CRISPR system, which does not require external processing elements.

TABLE 1
Active AsCpf1 repeat variants. Sequence regions
and corresponding nucleotides are shown in the
first five columns. SEQ ID NOs: 35-37 are each 19
nucleotides in length and SEQ ID NOs: 38 and
39 are each 20 nucleotides long.

| First | | | | Last | SEQ |
|---|---|---|---|---|---|
| 4 AAs | Stem1 | Loop | Stem2 | AA | ID NO. |
| aatt | tctac | tctt | gtaga | t | 35 |
| aatt | tctgc | tctt | gcaga | t | 36 |
| aatt | tccac | tctt | gtgga | t | 37 |
| aatt | tctac | tcgtt | gtaga | t | 38 |
| aatt | tctac | tcttt | gtaga | t | 39 |

These can be encoded in RNA-guided CRISPR gene drive systems to promote high penetrance and evolutionary stability. Guide RNAs may be expressed using a single Polymerase III or (less efficiently) Polymerase II promoter along with sequences promoting processing as needed, such as tRNAs, using previously described methods known to those in the art that are incorporated herein by reference (Xie et al 2015 PNAS doi:10.1073/pnas.1420294112, Mefferd 2015 RNA doi:10.1261/rna.051631.115, Port and Bullock bioRxiv doi:10.1101/046417). Alternatively, Cas9 and Cpf1 spacers may alternate with both nucleases expressed, causing Cpf1 to process the array into pairs of active guide RNAs, one corresponding to each nuclease. Alternatively, two guide RNAs may be expressed from a single Polymerase III promoter using 5-50 base pair linkages between the two guide RNAs. Alternatively, each guide RNA may be expressed from its own promoter, which may be a Polymerase III promoter. Suitable Polymerase III promoters with minimal homology are known to those in the art, e.g. U6, H1, and tRNA promoters (Port et al 2014 PNAS doi:10.1073/pnas.1405500111, Ranganathan et al 2015 doi:10.1038/ncomms5516).

### Design and Construction of RNA-Guided DNA Nuclease Gene Drive Elements

Methods of the invention, in part, include designing and constructing RNA-guided DNA nuclease gene drive elements that target multiple sequences within genes whose loss impairs successful gametogenesis and are active in the germline after the soma-germline division has been specified but before meiosis.

Gene drive elements spread most effectively when they are minimally costly to the organism. Targeting multiple sites within genes important for fitness can avoid creating drive-resistance alleles, but still creates a fitness cost due to the effects of losing such an important gene whenever repair occurs by the wrong mechanism (e.g. not homologous recombination).

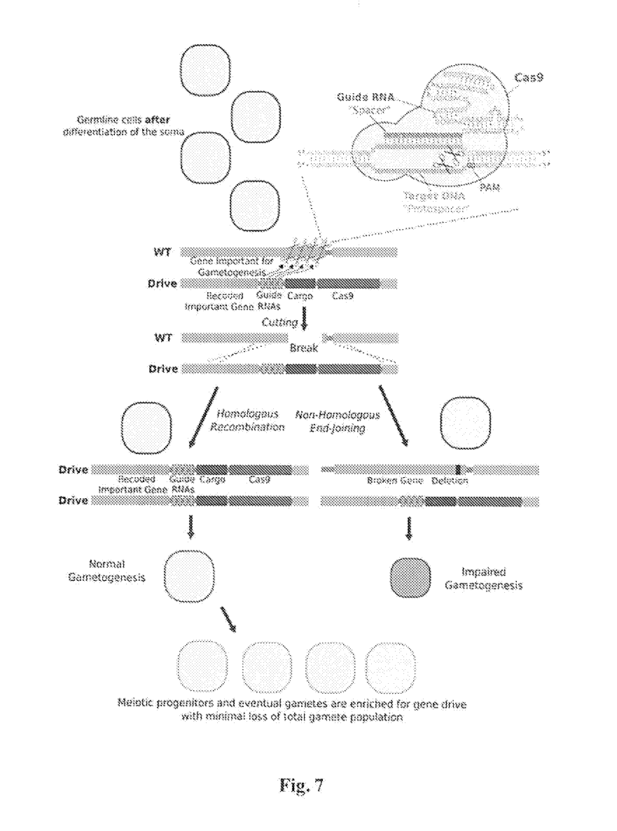

Methods of designing and constructing gene drives in which this fitness cost is reduced or eliminated by specifically targeting and recoding genes that are not just important for fitness, but are specifically important for the successful progression of gametogenesis, e.g. the production of sperm and/or eggs. Any event caused by such a drive element that expressed in the germline, and in some instances in the early germline, prior to meiosis impairs the ability of the cell to progress through gametogenesis (FIG. 7). [A non-limiting example of such a gene is hnRNP-GT in the mouse (Ehrmann, I., et al., Hum Mol Genet. 2008 Sep. 15; 17(18):2803-18; and others are known in the art.] Because other cells in which the drive element is correctly copied are not so impaired and compensate for the absence of the impaired cells that are lot, there is little if any loss in total gamete production, and hence little if any fitness cost to the drive element due to improper repair events. This design strategy permits efficient gene drive in organisms in which it might otherwise not be possible, and notably increase the efficacy in all others.

### Building and Using Serially Dependent 1-Dimensional Daisy Chains of Gene Drive Elements (Daisy Drive) Organisms

Methods of the invention, in part, include designing, constructing, and using serially dependent 1-dimensional daisy chains of gene drive elements (daisy drive) organisms (N−1 or generic) with an arbitrary number of elements such that the terminal element exhibiting drive encodes the only RNA-guided DNA nuclease such that any new element encoding its own guide RNAs can be added in order to alter or suppress populations, and of controlling the activity of the resulting drive system.

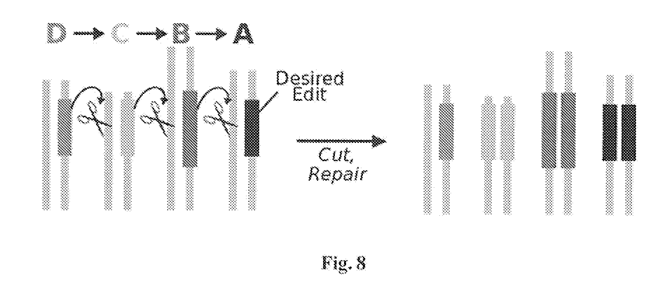

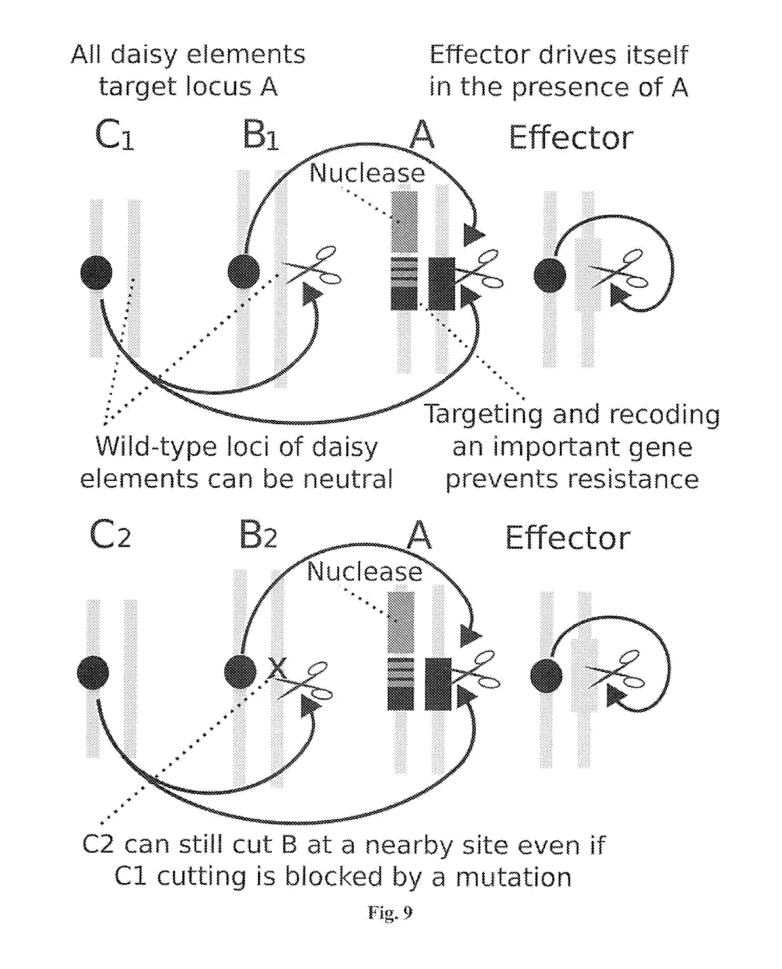

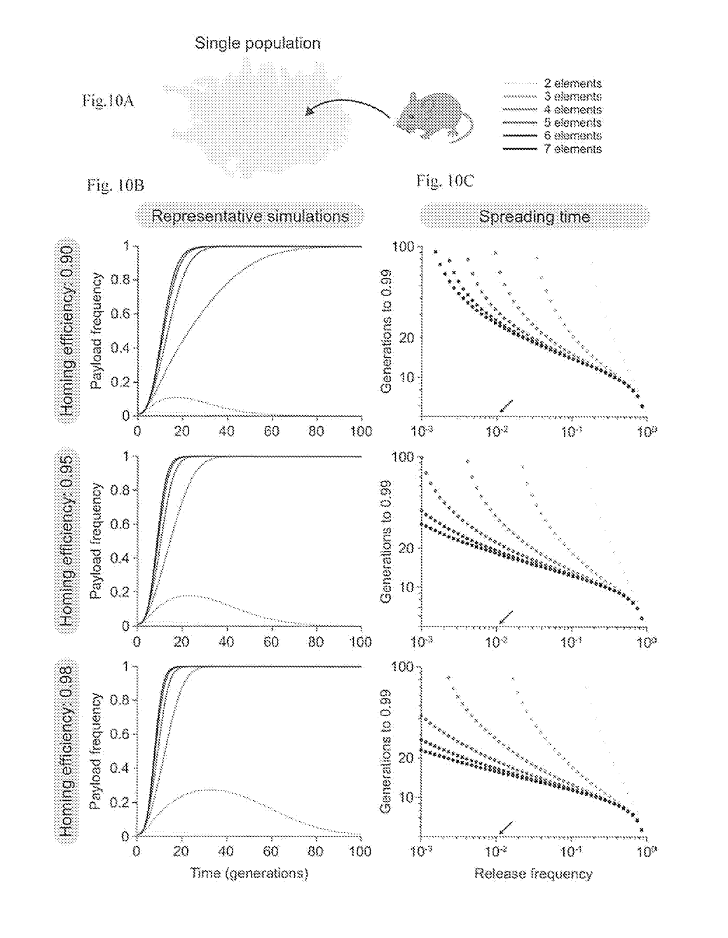

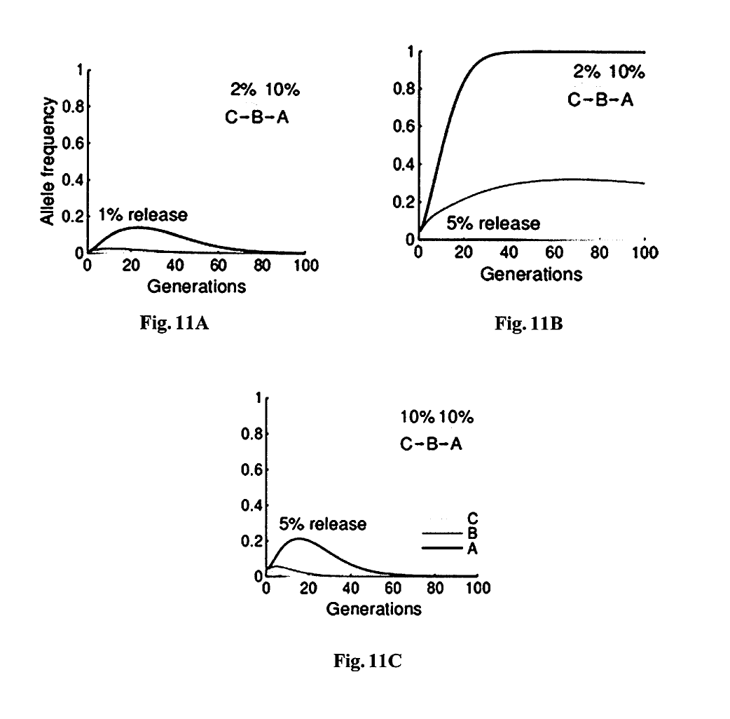

Some aspects of methods of the invention can be used to construct serially dependent CRISPR gene drive elements arranged in a daisy chain, which together form a “daisy drive” system (FIG. 8). They are arranged as a series of letters in the order opposite the alphabet, such that the terminal element is always “A”. Because the proximal element in the chain (e.g. C in a three-element daisy drive system) does not exhibit drive, its abundance is typically limited to the initial frequency at which it is released in the population, modulated by the fitness cost of all the daisy drive elements to the organism. The next element exhibits drive only when the proximal element is present, and so tends to lose the ability to exhibit drive swiftly (FIG. 9)

Recombination events between the elements of a linear daisy drive system have the potential to create a necklace of mutually dependent elements that can exhibit global drive (FIG. 5). Hence, homology between the elements must be minimized, which is accomplished using methods such as those set forth in: Example 1, Method 1.0 to identify highly divergent guide RNA sequences; Example 2, Method 2.3 to identify different promoters; and Method 3.1 to express multiple guide RNAs with minimal homology.

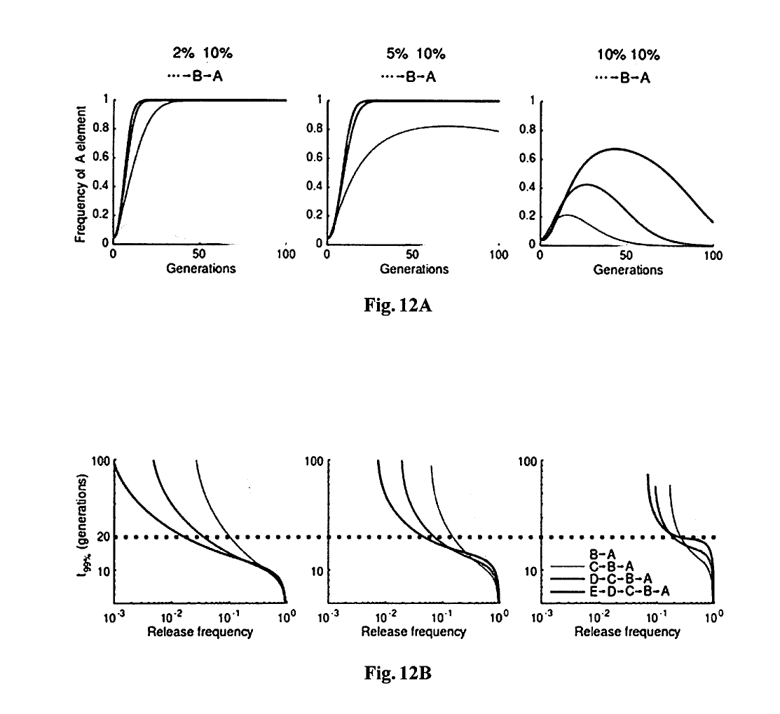

Models that were prepared predicted that daisy drives are more effective (e.g. they behave more like global self-sustaining drive systems) the more elements they have (FIG. 10). Constructing daisy drive organisms requires that one independent gene insertion event must occur for every element in the chain. Method have been determined that can be used to generate daisy drive chains capable of different purposes, such as the alteration of distinct genes or of population suppression, using the same base chain. Specifically, as described elsewhere herein, methods have been developed for designing and constructing a daisy chain gene drive organism containing N−1 elements, where N is the total number of elements in the chain desired, is generated such that the terminal element in that chain (hereby designated the B element) encodes the RNA-guided DNA nuclease. Subsequently, a) a new A element accomplishing the desired change, be it alteration or suppression, and also encoding guide RNAs enabling it to drive in the presence of the RNA-guided DNA nuclease, is added to the organism's genome directly by standard methods known to those in the art so as to create a complete N-element daisy drive organism, orb) a new A element accomplishing the desired change, be it alteration or suppression, and also encoding guide RNAs enabling it to drive in the presence of the RNA-guided DNA nuclease, is separately inserted into the genome of another organism of the same species, which then is crossed with the daisy drive line in the laboratory so as to create a complete N-element daisy drive organism, or c) the (N−1) element organisms are released into the environment to initiate a daisy drive effect that spreads the gene encoding the RNA-guided DNA nuclease through the local population, after which organisms encoding a desired “Z” element can be subsequently released to accomplish the desired effect, noting that while suppression will eliminate the RNA-guided DNA nuclease from the population, alteration can be accomplished multiple times in series or in parallel using different Z elements.

### Building and Using Serially Dependent 1-Dimensional Daisy Chains of Gene Drive Elements (Daisy Drive) Organisms

Methods of the invention, in part, include designing, building, and using serially dependent 1-dimensional daisy chains of gene drive elements (daisy drive) organisms wherein the terminal element that exhibits drive results in population suppression through either sex-biasing (via targeting a sex chromosome in the germline after the soma-germline division has been specified but before meiosis such that surviving gametes will produce individuals mostly of one sex) or genetic load, (via disrupting genes essential for viability or fertility in one or both sexes in the germline after the soma-germline division has been specified but before meiosis).

Methods of suppressing populations using endonuclease gene drive elements to bias the sex ratio or impose a genetic load have been previously described (Burt, A. 2003 Proc. Roy. Soc. Lond. B. 270,921-8; and Esvelt, K, et al., 2014 eLife:e03401, the content of each of which is incorporated herein by reference in its entirety). However, such gene drive elements are inherently self-sustaining and consequently pose risks to all populations of the target species anywhere in the world. New methods are provided herein that are used to limit population suppression to local rather than global populations by creating “daisy chain” gene drive elements that cause local population suppression.

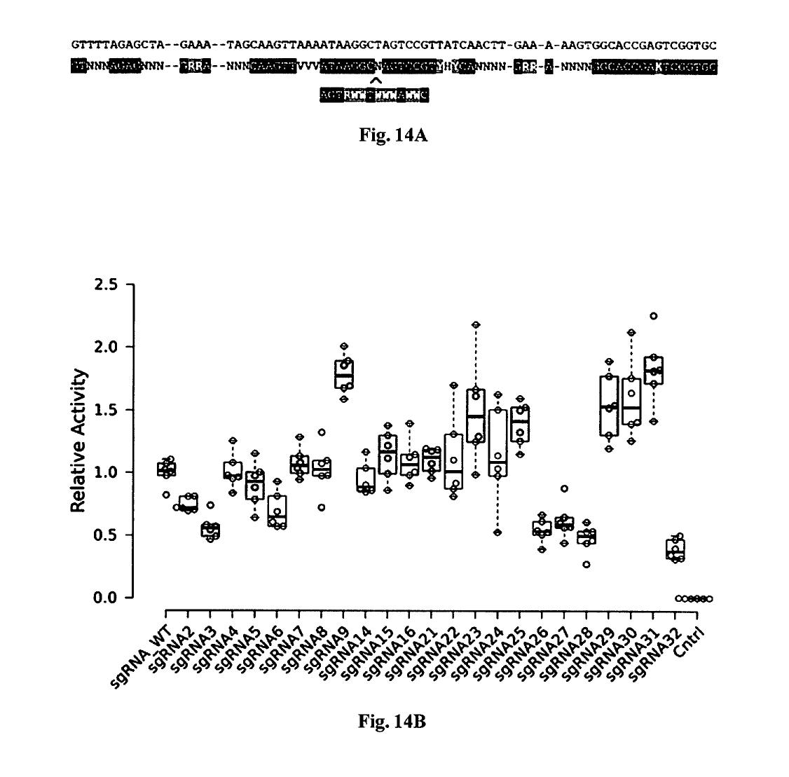

Specifically, methods are provided for the design and construction of a daisy drive chain of any length can be constructed in which the each element requires the prior link in order to drive, and the first element in the chain does not exhibit drive. By including a terminal element at position A that imposes genetic load (FIG. 14) or generates a sex-biasing effect, the daisy drive element suppresses the population in the area of release, but because it is a limited daisy drive rather than a self-sustaining drive, that effect will be limited to the area of release.

Any potential configuration of daisy drive elements can be adjusted using methods provided herein to induce a population suppression effect. For example, a daisy chain gene drive can be designed and constructed in which target effector element can replace and therefore eliminate a recessive gene that is important for viability or fertility as would a self-sustaining/global genetic load drive, or a daisy chain gene drive may be designed and constructed that includes multiple guide RNAs that target and disrupt such a gene. Alternatively, element A may be a standard daisy drive element (as described in Example 3, Method 3.0) that also encodes both guide RNAs targeting such loci for disruption as well as guide RNAs causing itself to drive. Alternatively, the A element or an effector element could include an extra copy of the single gene or set of genes that ensure the organism will develop a one particular sex in the relevant specie; for example, a single copy of the Sry gene in mice causes maleness. Alternatively, the A element or an effector element could include guide RNAs inducing the RNA-guided DNA nuclease to cut and eliminate a sex chromosome, thereby ensuring that nearly all offspring of A or A+effector element organisms are of one sex. These and other strategies for population suppression can be utilized in methods to design and construct daisy chain gene drives.

Methods of the invention, in part, include designing, constructing, and using serially dependent 1-dimensional daisy chains of gene drive elements (daisy drive) organisms with an arbitrary number of elements such that the terminal element targets and recodes a gene important for organismal fitness as it spreads in order to enable the subsequent alteration or suppression of exclusively the previously altered local population at a later date.

Altering a population with a daisy drive permits subsequent precision targeting of the introduced sequence with a global CRISPR gene drive system, which will not spread beyond the target population. This is a “precision drive” strategy. It is most effective if the “A” element or an effector element of the daisy drive alters a gene suitable for targeting with a suppression drive. Single stage, two-stage, and multiple-stage suppression daisy chain gene drive systems can be designed, constructed, and implemented using methods of the invention.

### Achieving Stable Population Suppression.

Methods of the invention, in part, include achieving stable population suppression by locating the first element in the daisy drive chain in a position unique to one sex and suppressing fertility or viability of the other sex. Daisy drive systems of the invention used directly for population suppression may experience a fitness cost limiting their potency. It is possible to ensure that the incidence of the daisy drive remains nearly proportional to the current population by reducing the fertility or viability of one sex while locating the first element of the daisy chain adjacent to a gene unique to the other sex.

For example, a simple C→B→A daisy drive might encode the guide RNAs of the C element adjacent to a male-determining gene (for example, but not limited to: the Nix gene within the M factor of the dengue vector Aedes aegypti) or a sex chromosome unique to males (for example, but not limited to: the Y chromosome in the malaria vector Anopheles gambiae). The RNA-guided DNA nuclease is encoded at a B element as is standard for a daisy drive. The A element would include guide RNAs that target and either disrupt or replace female fertility or viability genes. Alternatively, guide RNAs disrupting these genes might be encoded on the B element leaving the A element without guide RNAs of its own.

As a result, daisy drive males inactivate the female fertility genes during gametogenesis. Their sons would always inherit the C element (as well as B and A thanks to drive) and would suffer minimal fitness penalty, allowing them to repeat the cycle as it occurred in their fathers. Daughters would inherit one copy of the B element and the A element. During gametogenesis, the A element would drive because of the presence of the B element, so all offspring of these daughters would inherit a broken copy. If the other parent is a daisy drive male, their daughters will be sterile, thereby suppressing the population.

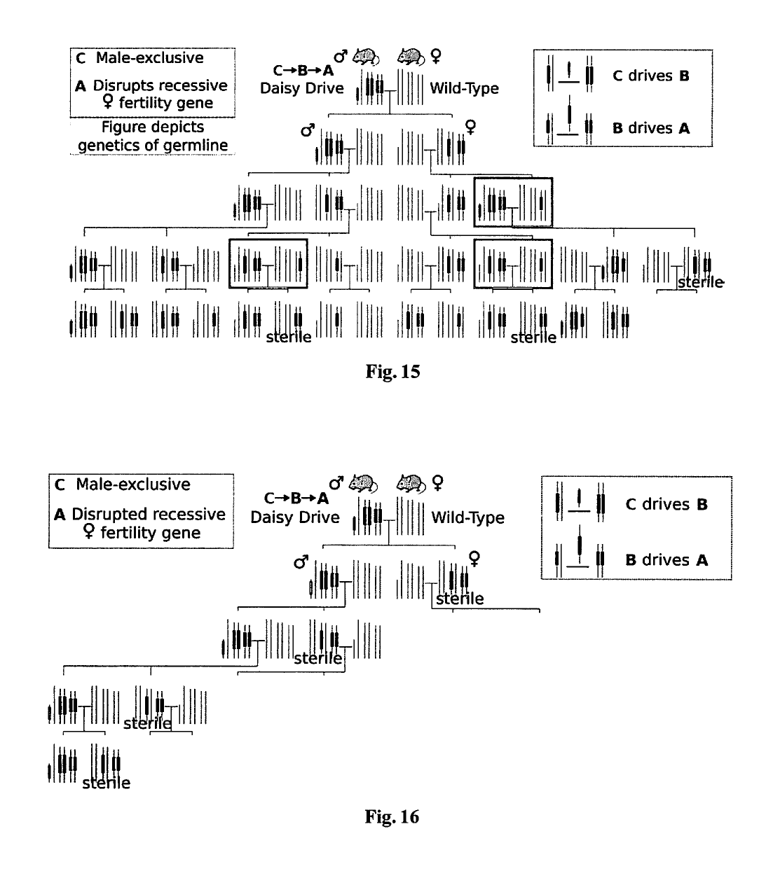

Methods of the invention, in part, include achieving stable population suppression with a daisy intermediate designed, constructed, and used to inactivate female fertility genes in a dominant manner. A variation on the above population suppression methods involves ensuring that the A element exhibits drive in the zygote, thereby ensuring that any female inheriting a single copy of the B element is sterile (or nonviable). This is achieved by arranging for the RNA-guided DNA nuclease encoded in B to be expressed in the zygote and/or the early stages of development. This will cause it to disrupt the wild-type allele of the A element inherited from the other parent, resulting in sterile or nonviable females. Because the fitness cost to males will be minimal, the introduction of males of this type will cause immediate population suppression proportional to the fraction of daisy drive males (FIG. 15). This approach is often necessary because there are few genes whose loss causes dominant sterility in a sex.

Another variation on the above population suppression methods is illustrated in FIG. 16. FIG. 16 illustrates a daisy drive that imposes a genetic load on female fertility as designed and constructed in Example 4, Method 4.0, but one in which the proximal element (C in this case) is embedded within a male-exclusive genetic element to mitigate the fitness cost as set forth in Example 6, Method 6.0. Rectangles highlight mating events that trigger sterility in female offspring.

Designing and Constructing Daisy Drive Elements in which Guide RNAs are Embedded within Introns of Target Genes.

Methods of the invention, in part, include designing, constructing, and using daisy drive elements in which guide RNAs are embedded within introns of target genes. Some genes may not be amenable to recoding at the 3′ end, or to having their 3′UTR replaced. An alternative method has been developed in which the guide RNAs are encoded within the gene itself. This is most effective when the gene is highly transcribed; fortunately, most haploinsufficient genes chosen as daisy drive targets are ribosomal and are consequently some of the most highly expressed in the cell. However, guide RNAs must be produced from these transcripts without disrupting the function of the gene. A solution has been developed that includes embedding the guide RNAs within introns, separated by tRNAs for efficient processing. The tRNA-processing method has been shown to enable high nuclease activity in fruit flies when driven by strong polymerase II promoters (http://dx.doi.org/10.1101/046417); ribozyme-based processing (not suitable for daisy drive due to repetitiveness) works efficiently from within introns (http://dx.doi.org/10.1016/j.molce1.2014.04.022). To ensure that the guide RNAs are copied efficiently, the target wild-type gene must be cleaved on both sides of the intron.

### Building Evolutionarily Unstable Yet Robust Drive Systems Through Redundancy.

Methods of the invention, in part, include designing, constructing, and using homing-based gene drive systems that are not vulnerable to drive-resistant alleles that block drive copying and thus prevent the spread of the drive system. These alleles are generated naturally whenever the endonuclease cut is repaired by non-homologous end-joining, which can create indels or point mutations at the target site that block subsequent cutting. This is why evolutionarily stable drives target multiple sites within genes important for fitness.

The invention in part also includes methods to identify highly active guide RNA sequences that share minimal homology that may be included in a daisy chain gene drive system of the invention, and may enable evolutionary stable daisy drive as well as global CRISPR gene drive. However, it is possible to affect large numbers of organisms even without evolutionary stability. A typical rate of NHEJ repair is 5% (Gantz, V. & Bier, E. 2015 Science 24 April: Vol. 348, Issue 6233, pp. 442-444; Gantz, V. et al., 2015 PNAS Vol. 112 no. 49 E6736-E6743; and Hammond, A. et al., Nat Biotechnol. 2015 Dec. 7; doi:10.1038/nbt.3439, each of which is incorporated herein by reference in its entirety). Thus, at minimum 5% of the population will be unaffected by the drive system; the share will decline as natural selection favors the resistant alleles over the drive. This precludes suppression drive strategies, but may be acceptable for certain alteration-based requirements. One method of compensating is to build multiple evolutionarily unstable drive systems, each of which targets a single site, wherein each drive system can overwrite resistance alleles generated by the others, but cannot directly overwrite one another. This multiple-drive approach is less stable than using a single drive system that targets multiple sites within a sequence important for fitness because resistance alleles could accrue one by one in the former but not the latter, and also requires building many drive systems which complicates modeling and regulation. However, there is no need to target a sequence important for fitness.

Similar logic applies to daisy drive systems. Because a daisy drive system is not intended to spread indefinitely, each element will only be copied a fixed number of times. This limits the potential for drive-resistant alleles to emerge that block spread. However, this is counterbalanced by the increased number of elements that must be copied, which increases vulnerability to any one drive-resistant allele. Building multiple daisy drive elements at each position, all of which can overwrite resistance alleles that block the other versions, can compensate for this deficit.

### Methods for Enhanced Daisy Drive Precision

Methods of the invention, in part, include designing, constructing, and using gene drive systems that include a means of enhanced precision with respect to geographic regions and boundaries for the gene drive effects. Embodiments of such methods can be used to constrain the effects of a gene drive system within a region and/or boundary. As described elsewhere herein daisy drives of the invention may be used to produce regionally localized changes in organisms and populations. Enhancement methods of the invention can be used to increase regional precision of a released daisy chain gene drive. It will be understood that using certain embodiments of daisy chain gene drive systems in wild populations, can result in the presence of some organisms with genetic changes outside of the desired or intended regional space or area. In certain situations, it may be undesirable to have the released daisy chain gene drive present or active in an area that may be in proximity to an area of a consenting community in which release and presence is intended. One non-limiting example of a means to reduce and/or prevent the presence in an unintended region or area is the use of buffer zones within the consenting community. For example, a community may desire to utilize release of a daisy chain gene drive system in a first area, but may need to limit entry of the system into a second area, for example in an adjacent community that does not consent to the presence of the daisy chain gene drive system. The presence of a region of the first area that is a buffer region in which the daisy chain gene drive system is not released, can be used to protect the second area, but it may result in the buffer region of the first area lacking the desired effect of the daisy chain gene drive system.

Certain embodiments of daisy chain gene drive systems of the invention are referred to herein as “precision,” “precision containment,” or “enhanced precision” daisy chain drives or systems, terms that indicate that the daisy drives are designed in a manner that when they are released in wild populations there is a reduced presence of organisms with genetic changes resulting from the introduced daisy chain gene drive system in areas and regions that are outside of a desired or intended region or area, compared with the level and/or presence of organisms with the genetic changes resulting from an introduced daisy chain gene drive outside the desired or intended region or area following release into a wild population of a gene drive system that is not a precision gene drive system of the invention.

An additional strategy to increase precision of localization of a daisy chain gene drive system has now been developed. Embodiments of a precision containment method of the invention comprise combining daisy drive systems with underdominance methods in order to keep population-genetic boundaries clear and distinct, enabling them to closely conform to regional and area boundaries. In genetics, underdominance is a condition in which selection is against the heterozygote. In underdominance situations, the heterozygote is less fit than a homozygote and thus is selected against in a population or organisms. Precision containment methods of the invention that ensure that hybridization between wild-type and engineered organisms results in fewer progeny—will select against whichever version of organism is currently less common in the population, thereby keeping the engineered and wild-type populations pure.

Methods of the invention can be used to reduce the fitness of altered individuals within wild-type populations and wild-type individuals within altered populations, resulting in the boundary between these populations becoming sharper and more distinct. This allows the boundary to be adjusted to closely conform to one or more geographic, community, and desirable areas and boundaries by targeted releases of wild-type or daisy drive organisms. A key aspect of combining daisy drive systems with underdominance is to ensure that the underdominance effect only triggers when the daisy drive activity ceases. This is necessary because daisy drive organisms are always rare relative to wild-type when released; thus, if underdominance took effect immediately, the daisy drive organisms would be strongly selected against.

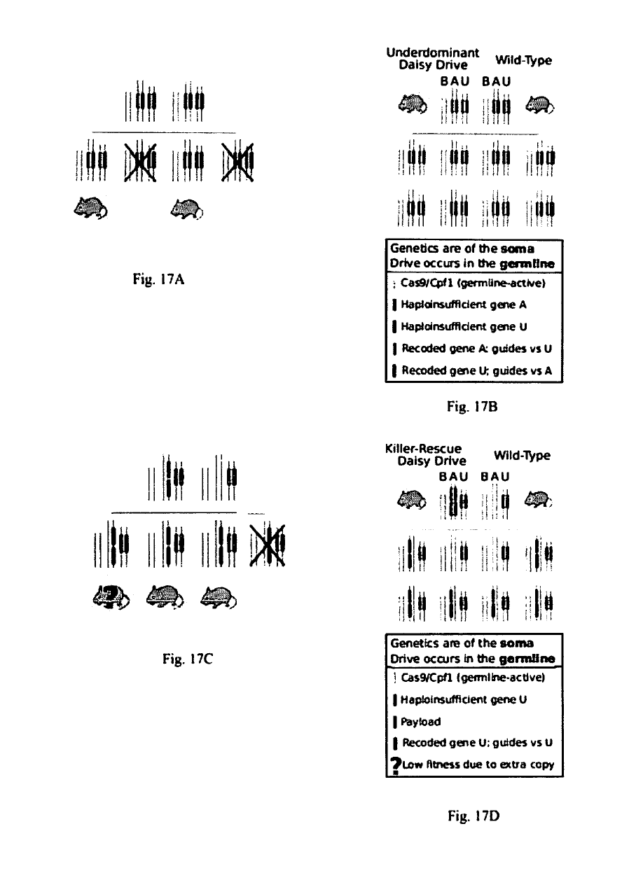

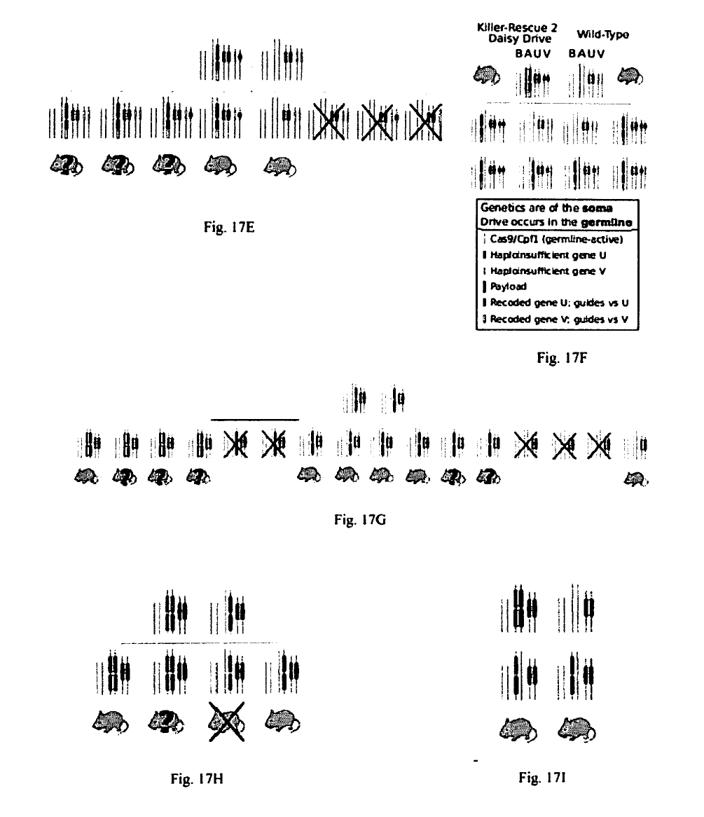

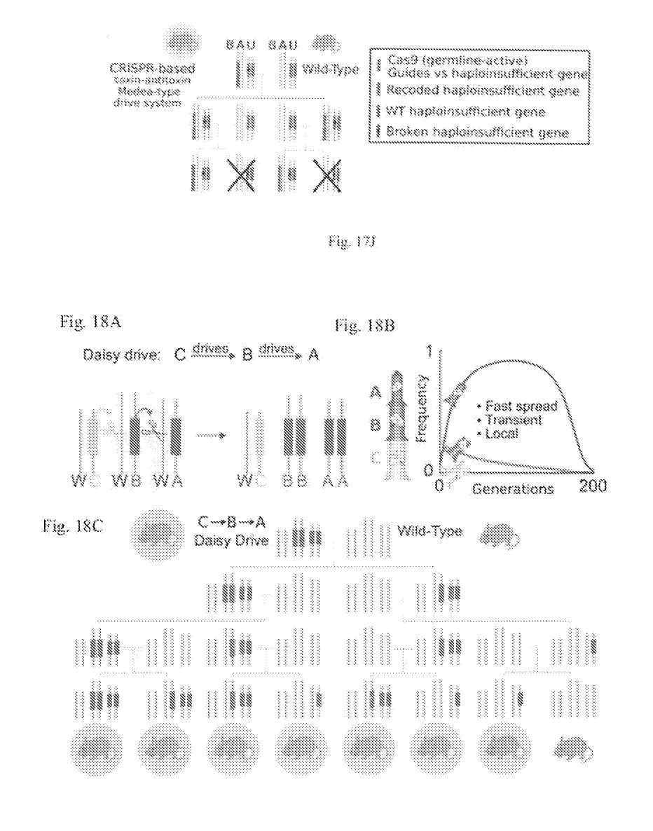

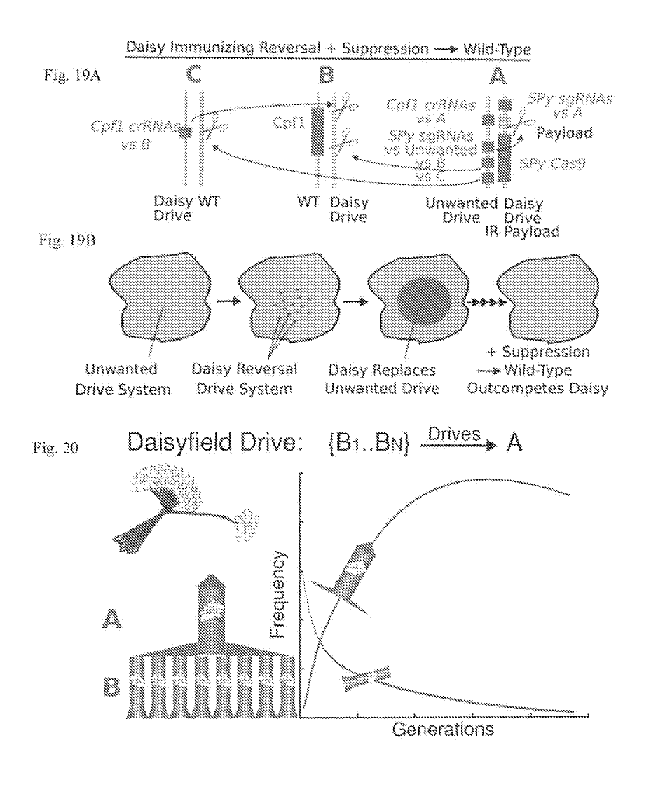

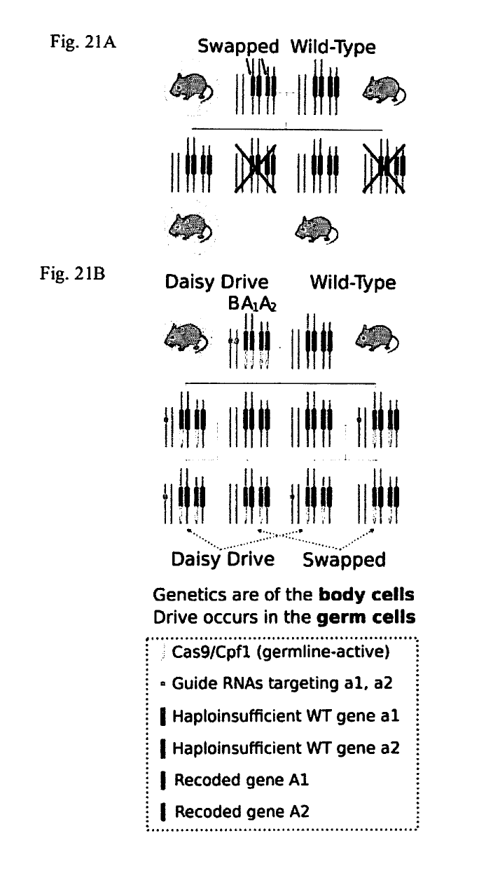

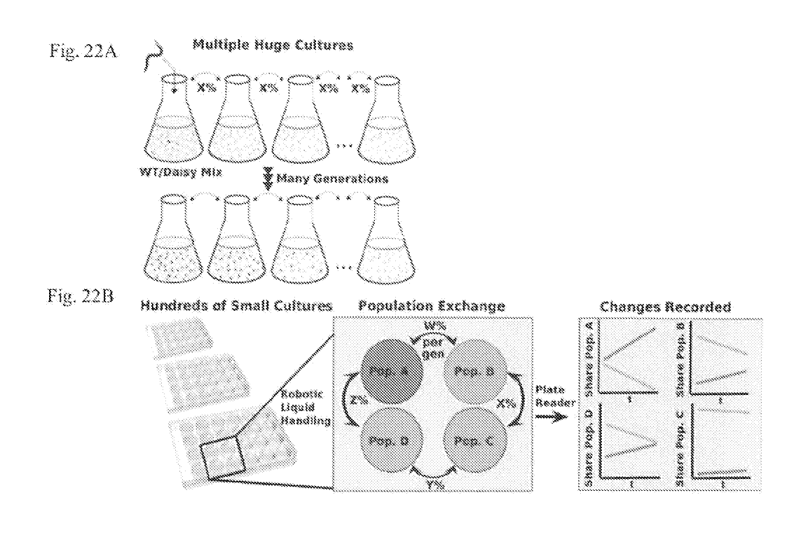

In some aspects of the invention, methods are provided that accomplish underdominance by creating a chromosomal rearrangement that swaps the positions of two or more essential genes in a cell in an organism. Normal chromosomal segregation during meiosis therefore assures that matings between heterozygotes and wild-types result in only 50% progeny survival (FIG. 17A). The same effect occurs when heterozygotes mate with homozygous altered organisms or even with other heterozygotes. Methods of the invention, in some aspects comprise swapping the locations of essential genes to result in an underdominance effect in subject and population of organisms. In some aspects of the invention, such organisms are released into a wild population as described elsewhere herein.

It is also possible to accomplish underdominance effect in population of organisms using methods of the invention in which one gene is not directly replaced with another (as described above), but rather methods in which guide RNAs are inserted that will eliminate the other gene and a re-coded copy that will rescue individuals that inherit it. Such methods of the invention can comprise including the inserted cassettes in different locations in the genome in one or more organisms. In some aspects of the invention, such organisms are released into a wild population as described elsewhere herein.

Underdominance can also be accomplished in daisy drive gene systems of the invention. As a non-limiting example, CRISPR-based underdominance daisy drive methods of the invention take advantage of the fact that a daisy drive payload element normally targets and recodes a gene important for fitness anyway, for example, a haploinsufficient gene. A non-limiting embodiment is shown in FIG. 17B. In this example, at least two such payload elements can be created (for example: A and U in FIG. 17B). Genetic locus A normally has haploinsufficient gene hA; while genetic locus U normally has haploinsufficient gene hU. In the daisy drive version, element A has guide RNAs targeting hU as well as a recoded copy, hU′, in place of the hA. Similarly, element U has guide RNAs targeting hA as well as a recoded copy, hA′, in place of hU. In other words, these elements catalyze the replacement of the wild-type gene at their own locus with a re-coded version of the other locus' gene. The genes swap positions. When the drive nuclease is present (element B), drive occurs in both places, thereby replacing hA with hU′ and hU with hA′. All offspring inherit one of each and consequently are guaranteed to be fine. But when there is no drive nuclease, i.e. the daisy drive has run out of genetic fuel (elements), offspring will inherit either hA or hU′ and either hU or hA′, meaning half of them will end up lacking a working copy of a haploinsufficient gene and consequently be very unfit. In this embodiment of the invention, underdominance occurs only when the daisy drive runs out of elements and stops.

Another non-limiting example of an underdominance daisy drive method of the invention is RNAi-based toxin-antitoxin underdominance daisy drive methods. For example, Akbari et al 2013 Current Biology Volume 23, Issue 8, p 671-677, the content of which is incorporated herein by reference in its entirety, describes a two-locus UDmel method in which maternal deposition of inhibitory RNAi molecules targeting an essential gene renders progeny nonviable unless they inherit a recoded copy of that gene that is not inhibited. Components, sequences, and methods disclosed by Akbari et al., including but not limited to the uDmel locus, can be used in certain embodiments of daisy drive underdominance systems and methods of the invention. For example, one UDmel locus can be incorporated into element A of a daisy drive, and the other locus into element U. As long as the daisy drive is active, all offspring will inherit the recoded copy and be fine; e.g. underdominance will not take place. Once the daisy drive runs out of elements, Mendelian segregation will occur, meaning not all offspring will inherit the protective copy. Males will transmit both copies as normal.

Another non-limiting example of an RNAi-based toxin-antitoxin underdominance daisy drive method of the invention includes RNAi-based toxin-antitoxin underdominance without a maternal effect. An embodiment of such a method of the invention may include in a daisy drive system of the invention, a copy of an underdominance cassette that knocks down a haploinsufficient gene via RNAi and provides a recoded copy, in payload element A, and another in payload element U. An example of an underdominance cassette that may be used in an RNAi-based toxin-antitoxin underdominance daisy drive method of the invention is set forth in Reeves et al., 2014 PLoS, http://dx.doi.org/10.1371/journal.pone.0097557, the content of which is incorporated herein by reference in its entirety. Components, sequences, and methods disclosed Reeves et al., (for example in FIG. 1, page 1-2, etc.) can be used in certain embodiments of daisy drive underdominance systems and methods of the invention. For example, some embodiments of RNAi-based toxin-antitoxin underdominance daisy drive systems of the invention include at least one copy of a cassette such as that disclosed in Reeves, which will knock down a haploinsufficient gene via RNAi and will provide a recoded copy in payload element A, and another in payload element U. As long as all offspring inherit a recoded copy of A and U because the drive is active, the offspring are viable. When the embodiment of the underdominance daisy drive of the invention is no longer active, any offspring with wild-type that do not inherit a copy of both the A and U elements will not be viable. This is consequently more effective as only ¼ of the offspring will survive.

Another non-limiting example of a toxin-antitoxin underdominance daisy drive method of the invention in the zygote of an organism comprises using a zygotically active form of CRISPR (e.g. not using the germline-active form employed in the daisy drive). In certain embodiments of enhanced precision daisy chain systems and methods of the invention, instead of relying on RNAi to suppress expression of essential or haploinsufficient genes, CRISPR is used as a toxin to much more reliably disrupt the essential or haploinsufficient genes. In some embodiments of such systems and methods, the antitoxin is a recoded version of the targeted gene that is not disrupted by the CRISPR system.

FIG. 17A-J illustrates certain embodiments of the above-described systems. FIG. 17A-J provides schematic diagrams illustrating embodiments of underdominance, CRISPR-based killer rescue systems, and other killer-based rescue systems of the invention. FIG. 17C illustrates a CRISPR-based killer-rescue system, also referred to as: a toxin-antitoxin system, generated by inserting a copy of a haploinsufficient gene next to the payload and disrupting the wild-type copy elsewhere in the genome. Offspring that inherit a disrupted version without the new copy perish. Offspring that inherit more than the normal two copies may or may not be highly unfit due to the extra expression; if they are reasonably fit then the payload will spread to a limited extent. The net effect is a form of underdominance. FIG. 17D illustrates a killer-rescue system generated by a daisy drive system, which encodes the germline-expressed nuclease in the B element, a recoded copy of the haploinsufficient gene along with the payload in the A element, and guide RNAs that disrupt the wild-type copy in the U locus. Daisy drive propagation occurs as normal because all offspring inherit a recoded copy and a broken copy until the nuclease is no longer present. At this point the killer-rescue/toxin-antitoxin system becomes active and selects for homozygosity at A and U. FIG. 17E illustrates a more powerful killer-rescue system for which heterozygotes produce fewer progeny that is generated by encoding two different copies of a haploinsufficient gene next to the payload and disrupting the wild-type copy. Offspring that inherit either disrupted version without the payload perish. Offspring that inherit more than the normal two copies may or may not be highly unfit due to the extra expression; this may cause the payload to spread if they are reasonably fit. The net effect is a stronger form of underdominance. FIG. 17F illustrates that a stronger killer-rescue system can also be generated by a daisy drive system so that it manifests after the drive halts. The stronger underdominance is more effective at selecting for homozygosity at A, U, and V (the locus encoding the second haploinsufficient gene). FIG. 17G-I provides diagrams of family trees demonstrating the underdominance effect and possible limited spread caused by the killer-rescue/toxin-antitoxin system. FIG. 17J illustrates a CRISPR-based toxin-antitoxin system that generates a Medea effect: any offspring that do not inherit the Medea element perish due to lack of a haploinsufficient gene. Because it is expected that Medea elements will be self-sustaining in the event of density-dependent selection, in some embodiments of the invention, they are generated without adding a daisy drive. In certain circumstances, a non-limiting example of which is when a goal is to release very few organisms in order to exceed the threshold level for continued spread, a daisy drive system can be added. Adding a daisy drive system can be done by including another element (B) that encodes guide RNAs that drive the Medea element (not shown). Each of the above-described systems of the invention, certain embodiments of which are illustrated in FIG. 17A-J, can be prepared and utilized using methods, components, and elements as described herein. In some aspects of the invention, methods of the invention may also include art-known procedures and elements.

### Quorum Aspects

Embodiments of a gene drive systems of the invention are designed to alter wild populations in a manner that ideally: exclusively affects organisms within the political boundaries of consenting communities, and are capable of restoring any engineered population to its original genetic state. The invention, in part, includes daisy quorum drive systems that meet these criteria by combining daisy drive with underdominance. A daisy quorum drive system of the invention is predicted to spread through a population until all of its daisy elements have been lost, at which point its fitness becomes frequency dependent: mostly altered populations become fixed for the desired change, while engineered genes at low frequency are swiftly eliminated by natural selection. The result is an engineered population surrounded by wild-type organisms with limited mixing at the boundary. Releasing large numbers of wild-type organisms or a few bearing a population suppression element can reduce the engineered population below the quorum, triggering elimination of all engineered sequences. In principle, the technology can restore any drive-amenable population carrying engineered genes to wild-type genetics. Daisy quorum systems of the invention enable efficient, community-supported, and genetically reversible ecological engineering.

The following examples are provided to illustrate specific instances of the practice of the present invention and are not intended to limit the scope of the invention. As will be apparent to one of ordinary skill in the art, the present invention will find application in a variety of compositions and methods.

### EXAMPLES

### Example 1

Methods to design and construct RNA-guided gene drives based on CRISPR/Cas9 have been developed. Use of the methods singly, in combination of two or more, and in combination of one or more with other design methods for gene drives permits daisy chain gene drives to be designed, constructed, and used. Daisy chain gene drives prepared using one or more methods described herein are included in cells, cell lines, and/or organisms.

Daisy chain gene drives designed using methods provided herein are used to address otherwise intractable ecological problems, with a level of safety inherent in their design, that reduces or eliminates a likelihood of global of daisy chain gene drive organisms that are released into the wild. Daisy gene drive elements and systems designed and/or constructed using methods provided herein are used to reduce instances and control vector-borne and parasitic diseases such as, but not limited to: malaria, schistosomiasis, dengue, and Zika.

Gene drive elements and systems designed and/or constructed using one or more methods provided herein, include molecular constraints that when included in an organism or population of organisms, limit geographic spread in a tunable manner. Gene drive design and construction methods set forth herein are used in ecological engineering by enabling local communities to make decisions concerning their own environments.

Methods of Designing and Constructing RNA-Guided DNA Nuclease Gene Drive Elements that Target Multiple Sequences but do not Themselves Encode Repetitive Elements.

Endonuclease gene drive systems continually create alleles that they cannot replace whenever nuclease-cut DNA is repaired by non-homologous or microhomology-mediated end-joining or a similar pathway in a manner that mutates the recognition site of the endonuclease. If the resulting mutant allele confers higher fitness to the organism than the drive system, natural selection will favor the mutant drive-resistant allele, preventing the drive system from ever reaching fixation and eventually leading to its elimination from the population. Targeting a gene important for fitness can reduce the frequency at which this occurs, but synonymous mutations or non-synonymous mutations, in-frame insertions, or deletions could still preserve function and outcompete the drive system.

A reliable method of overcoming this problem is to program the endonuclease to cut multiple nearby sites within a gene important for fitness such that any repair method that does not involve homologous recombination (and hence copying of the drive system) deletes the portion of the gene between the cut sites and consequently creates a loss-of-function mutation that is more costly than the drive (Esvelt et al 2014 http://dx.doi.org/10.7554/eLife.03401). Targeting multiple sites also reduces the chance of each cut being repaired to create a minimally costly mutation independently; the more sites targeted, the lower the chance of any allele acquiring resistance to each cut. However, this multi-site targeting must be accomplished without introducing repetitive sequences into the drive system, as internal repeats frequently lead to internal recombination and instability of the drive cassette (Simoni et al Nucleic Acids Research 2014 http://dx.doi.org/10.1093/nar/gku387). Such an event could inactivate the drive system, which reduces its overall efficiency, or worse yet might reduce or eliminate its ability to target multiple sites, thereby promoting the emergence of resistance alleles which in turn could lead to the serial acquisition of resistance to all cut sites.

CRISPR systems can readily target multiple sites using different guide RNAs, but each of these must be separately encoded in a way that does not permit internal recombination.

Methods are provided that enable targeting multiple sites by identifying sets of guide RNAs with very little homology to one another. Additionally, a set of highly active guide RNA sequences is disclosed that have been verified to function with the most commonly used CRISPR system, that of S. pyogenes. These can be encoded in RNA-guided CRISPR gene drive systems to promote high penetrance and evolutionary stability. Guide RNAs are expressed using a single Polymerase III or (less efficiently) Polymerase II promoter along with sequences promoting processing, such as tRNAs, using previously described methods known to those in the art that are incorporated herein by reference (Xie et al 2015 PNAS doi:10.1073/pnas.1420294112, Mefferd 2015 RNA doi:10.1261/rna.051631.115, Port and Bullock bioRxiv doi:10.1101/046417). Alternatively, two are expressed from a single Polymerase III promoter using 5-50 base pair linkages between the two guide RNAs. Finally, each guide RNA is expressed from its own promoter, which may be a Polymerase III promoter. Suitable Polymerase III promoters with minimal homology are known to those in the art, e.g. U6, H1, and tRNA promoters (Port et al 2014 PNAS http://dx.doi.org/10.1073/pnas.1405500111).

A problem may arise because of the length of the portion of the guide RNA sequence that is recognized by the CRISPR system, which may be Cas9 from S. pyogenes. This portion is over 60 bp in length, which is more than enough for internal recombination (Mali et al 2013 http://dx.doi.org/10.1126/science.1232033). Recombination was identified as undesirable in gene drives.

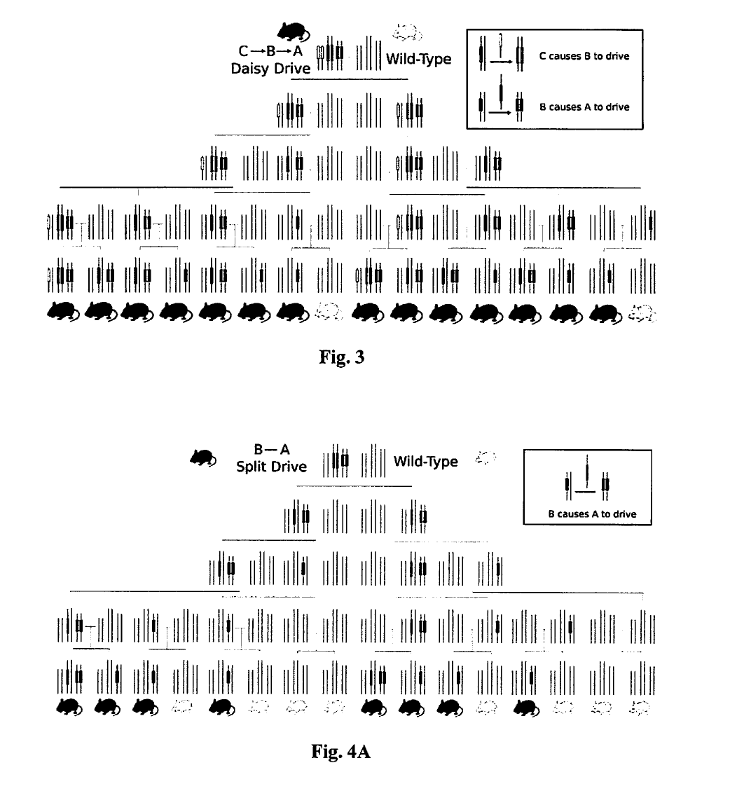

The known crystal structure (Nishimasu et al 2014 Cell http://dx.doi.org/10.1016/j.ce11.2014.02.001) and data on guide RNAs from closely related CRISPR systems (and synthetic variants) (Briner 2014 Molecular Cell http://dx.doi.org/10.1016/j.molce1.2014.09.019) that can be recognized by S. pyogenes Cas9 was used to identify candidate regions thought to be more or less important for guide RNA recognition. A set of guide RNAs was prepared using Method 1.0 (FIG. 2) and their activity was measured their activity to the “wild-type” sgRNA using a transcriptional activation assay (FIG. 3) (Mali et al 2013 Nature Biotechnology http://dx.doi.org/10.1038/nbt.2675).

A similar set of guide RNAs with minimal homology is created for any given CRISPR system through equivalent means. If nothing is initially known of the relevant dependencies, the relevant information is gleaned by performing structural studies similar to those referenced, or through a library-based approach (Method 1.1) followed by design and assaying (Method 1.0). Method 1.0 permits rapid preparation of DNA sequences capable of expressing multiple guide RNAs. Methods of the invention permit rapid identification and preparation of repetitive sequences, which was not previously possible.

Method 1.0—Creating Highly Divergent Guide RNA Variants with Minimal Homology to One Another.
(1) All known relevant information was used to create a list or map of the guide RNA denoting every possible individually accepted change throughout the structure. If there is insufficient information, Method 1.1 can be used to generate the relevant dataset.
(2) Several dozen elements were designed that combined permutations of these permitted changes so as to minimize the length of sequences shared between elements. In this context, the term “elements” means the backbone of the guide RNA sequence recognized by the nuclease that is capable of directing the nuclease to cut a target sequence.
(3) Activity of these designed sequences was measured e.g. by a transcriptional activity reporter assay as detailed below, or using a selection method as detailed in Method 1.2.
(4) All divergent guide RNA sequences that retained high activity were identified and recorded. These divergent guide RNA sequences are suitable for, and are used for, constructing evolutionarily stable homing-based gene drive systems that target multiple sites to overcome the evolution of mutations that block cutting. Because the divergent sequences dramatically reduce the chance of recombination between homologous sequences within the drive cassette, which is a major problem for highly repetitive drive cassettes (Simoni et al Nucl. Acids Res. 2014 http://dx.doi.org/10.1093/nar/gku387), the resulting drive systems are stable. They are also used for the construction of functional daisy drive system homology between elements to avoid recombination events that could lead to global drive activity, as detailed in Example 3, Method 3.0. These guide RNA sequences are also useful for synthesizing DNA sequences encoding multiple guide RNAs for standard multiplexing experiments involving CRISPR gene editing and regulation.
(5) FIGS. 6 and 14 detail a set of highly divergent guide RNAs that were designed and prepared and indicates their activity relative to the most commonly used guide RNA for the RNA-guided DNA nuclease Cas9 from S. pyogenes. Activity was determined using the fluorescent reporter assay detailed below Method 1.1. It has previously been very difficult to synthesize repetitive sequences, which has precluded attempts to quickly make DNA sequences capable of expressing multiple guide RNAs. Methods of the invention permit rapid identification and preparation of repetitive sequences that are used in daisy chain gene drives and other gene drives.

### Method 1.1—Library-Based Guide RNA Interrogation

(1) Two libraries are created. One is a randomized library of guide RNA sequences averaging 1-5 mutations per member and the second is a targeted library in which the base pairs in predicted hairpins are replaced with alternative base pairs that preserve the predicted hairpin structure (e.g. G-C pairs are replaced by C-G, A-T, T-A, G-T, and T-G) or create a mispair (e.g. C-C). These libraries can be generated by methods known to those in the art or synthesized as oligonucleotides by known commercial suppliers (e.g. CustomArray).
(2) Using a plasmid in which the protospacer sequence targeted by the spacer in the guide RNAs is directly adjacent to the sequence encoding the guide RNAs such that activity leads to cutting of the plasmid, transform bacteria or transfect eukaryotic cells that also express either active or inactive Cas9, perform high-throughput sequencing (such as but not limited to: Illumina MiSeq or HiSeq methods) of the plasmid sequences encoding the guide RNAs, and identify the most active variants as those most thoroughly depleted when Cas9 is active (e.g. using the method of Esvelt et al 2013 Nature Methods http://dx.doi.org/10.1038/nmeth.2681).
(3) Alternatively, two protospacer sites are encoded, and the region containing both as well as the guide RNA is PCR-amplified, the resulting amplicons are size-selected for those lacking the sequence between the protospacers, and are sequenced to identify those active enough to cut both sites. See Example 2, Method 2.3.
(4) Alternatively, transcriptional activation assay with a fluorescent reporter is used as is detailed in Method 1.2 and fluorescence-assisted cell sorting is used the guide RNAs that result in the highest levels of transcriptional activation are identified.
(5) All instances in which substituting bases in a hairpin retains activity are noted.
(6) All instances in which mutating a single base preserves activity are noted.
(7) All instances in which adding or removing a base preserves activity are noted.

### Method 1.2 Measuring Guide RNA Activity Via Transcriptional Activation Reporter Assay

Methods to measure and determine activity of candidate guide RNAs were designed and tested.

(1) Cells are grown using standard conditions (for example, HEK293T cells were grown in Dulbecco's Modified Eagle Medium (Life Technologies) fortified with 10% FBS (Life Technologies) and Penicillin/Streptomycin (Life Technologies), incubated at a constant temperature of 37° C. with 5% CO₂).
(2) The cells were split into multi-well plates, divided into approximately 50,000 cells per well and then transfected with plasmids encoding:

(a) dCas9-VPR or an equivalent dead-nuclease transcriptional activator variant of the RNA-guided DNA-binding protein nuclease matching the candidate guide RNAs to be tested,

(b) the candidate guide RNA to be evaluated,

(c) a reporter plasmid comprising a minimal promoter and one or more protospacer binding site upstream of a gene encoding a fluorescent protein, and

(d) a control plasmid expressing a different fluorescent marker gene as a transfection control marker.

(3) The transfections were carried out using standard methods, (for example, using 20 of Lipofectamine 2000 (Life Technologies) with 200 ng of dCas9 activator plasmid, 25 ng of guide RNA plasmid, 60 ng of reporter plasmid and 25 ng of EBFP2 expressing plasmid. The reporter plasmid was a modified version of addgene plasmid #47320, a reporter expressing a tdTomato fluorescent protein adapted to contain an additional gRNA binding site 100 bp upstream of the original site, the activator is da tripartite transcriptional activator fused to the C-terminus of nuclease-null Streptococcus pyogenes Cas9).
(4) After transfection, the cells were analyzed using flow cytometry to measure activity, and any cells that didn't fluoresce due to the presence of the transfection control marker were ignored.
(5) Optionally, if a library of guide RNAs is assayed at the same time, fluorescent-assisted cell sorting (FACS) is used to sort for plasmids encoding highly active guide RNAs which are then sequenced to identify.

### Example 2

Methods for Designing and Constructing RNA-Guided DNA Nuclease Gene Drive Elements that Target Multiple Sequences within Genes Whose Loss Impairs Successful Gametogenesis and are Active in the Germline after the Soma-Germline Division has been Specified but Before Meiosis.

Gene drive elements spread most effectively when they are minimally costly to the organism. Targeting multiple sites within genes important for fitness can avoid creating drive-resistance alleles, but still creates a fitness cost due to the effects of losing such an important gene whenever repair occurs by the wrong mechanism (e.g. not homologous recombination). Previous studies have proposed and more recently demonstrated, or at least attempted to demonstrate, population suppression drive elements that are not active in the embryo or the soma, only in the germline (Burt, A. 2003 Proc. Roy. Soc. Lond. B. 270,921-8; Hammond et al 2015 Nature Biotech).

Methods of designing and constructing gene drives in which this fitness cost is reduced or eliminated by specifically targeting and recoding genes that are not just important for fitness, but are specifically important for the successful progression of gametogenesis, e.g. the production of sperm and/or eggs. Any event caused by such a drive element that expressed in the germline, and in some instances in the early germline, prior to meiosis impairs the ability of the cell to progress through gametogenesis (FIG. 7). [A non-limiting example of such a gene is hnRNP-GT in the mouse (doi:10.1093/hmg/ddn179); and others are known in the art.] Because other cells in which the drive element is correctly copied are not so impaired and compensate for the absence of the impaired cells that are lot, there is little if any loss in total gamete production, and hence little if any fitness cost to the drive element due to improper repair events. This design strategy permits efficient gene drive in organisms in which it might otherwise not be possible, and notably increase the efficacy in all others.

Method 2.0—Building Evolutionarily Stable Gene Drive Systems with Minimal Fitness Cost.
(1) A gene is chosen that is known to be haploinsufficient for normal cell growth, e.g. one wherein a single copy is insufficient for normal growth and division.
(2) If no such gene is known: a gene is chosen that encodes a ribosomal protein, most of which are haploinsufficient. Assays are performed for haploinsufficiency in the germline via Method 2.1 below as needed.
(3) A gene is identified that is first expressed exclusively in the germline after soma-germline differentiation in the organism of interest. Assays are performed for expression timing via Method 2.2.
(4) The identified gene's promoter/enhancer/3′UTR is used to drive expression of the RNA-guided DNA-binding protein nuclease (e.g. Cas9 or equivalent) in a gene drive cassette, e.g. one that also encodes guide RNAs targeting the equivalent wild-type locus, where the guide RNAs are expressed from a promoter such as one identified using Method 2.3. Optionally, the nuclease is fused to a fluorescent protein (e.g. GFP) using 2A peptide tag and use fluorescent imaging of the embryo and it is verified that expression is germline-specific and occurs at the correct developmental stage.
(5) Measurement is performed to determine lifetime fertility of organisms encoding the candidate drive cassette when mated to wild-type partners as compared to wild-type/wild-type pairings to verify that there is no loss of reproductive fitness. Offspring are screened by PCR to identify any heterozygotes in which the drive has not been copied.

### Method 2.1—Assaying a Gene for Haploinsufficiency in the Germline.

(1) A strain of transgenic organisms is created in which an RNA-guided DNA-binding protein nuclease is expressed exclusively in the germline after soma-germline differentiation (see Methods 2.0, 2.2).
(2) One or more strains of transgenic organisms are created in which a single guide RNA targeting the coding region of the candidate haploinsufficient gene is expressed under a polymerase III (e.g. U6) promoter, which in some cases is one identified using Method 2.3.
(3) The two strains are crossed to create a heterozygous line in which the target gene is cut in germline cells just after soma-germline differentiation.
(4) The resulting hybrids are mated to wild-type organisms.
(5) The candidate haploinsufficient genes in the offspring zygotes or embryos, or the gametes of the original organism, are sequenced. If the gene is in fact haploinsufficient in the germline, all offspring or gametes should have intact copies resulting from cells in which the nuclease did not cut or copies with mutations that do not significantly impair the function of the gene.
Method 2.2—Expressing Genes Exclusively in the Germline after Soma-Germline Differentiation.
(1) If the organism is amenable, embryos are dissected to isolate germline cells and a full transcriptome sequencing analysis is performed. Candidate genes are chosen that are identified as expressed exclusively in the germline after the soma-germline differentiation step.
(2) If transcriptome analysis is not possible, the method of Merritt et al (2008) Current Biology (10.1016/j.cub.2008.08.013) is carried out and promoters/enhancers/3′UTRs are tested for appropriate expression.

### Method 2.3—Identifying Highly Active Promoters for Guide RNA Expression.

(1) Into an organism or cell line expressing an RNA-guided DNA nuclease, DNA encoding one of a number of candidate promoters driving a guide RNA is delivered. This guide RNA should target one or ideally two sequences located just upstream of the promoter.
(2) DNA is extracted and purified and PCR used to amplify the target site(s) as well as the candidate promoter.
(3) If using two target sites, amplicons are size-select to those lacking the sequence between the sites are identified.
(4) Sequence to identify which candidate promoters most often cleaved the target site(s).
(5) Alternatively, the DNA is delivered into cultured cells of the target organism. The repeated sequences are positioned in such a way as to disrupt production of a fluorescent protein encoded on the same fragment. A second fluorescent protein is encoded as a marker for cells that have taken up the DNA. Fluorescence-assisted cell sorting (FACS) is used to enrich for cells expressing the second fluorescent protein but not the first one, indicative of successful cutting. Sequencing is performed and the most active promoters identified.

### Example 3

Provided are Methods of Building and Using Serially Dependent 1-Dimensional Daisy Chains of Gene Drive Elements (Daisy Drive) Organisms with an Arbitrary Number of Elements Such that the Terminal Element Exhibiting Drive Encodes the Only RNA-Guided DNA Nuclease Such that any New Element Encoding its Own Guide RNAs can be Trivially Added in Order to Alter or Suppress Populations, and of Controlling the Activity of the Resulting Drive System.

The self-propagating nature of global gene drive renders the technology uniquely suited to addressing large-scale ecological problems, but tremendously complicates discussions of whether and how to proceed with any given intervention. Technologies capable of unilaterally altering the shared environment require broad public support. Hence, ethical gene drive research and development must be guided by affected communities and nations to an extent unprecedented in the history of science. Attaining this level of engagement and informed consent becomes more challenging as the number of people affected grows.

A way to confine the spread of a gene drive element to local populations would greatly simplify community-directed development and deployment. Prior strategies (see for example: Gould et al 2008 Proc Roy Soc B doi:10.1111/j.1558-5646.2007.00298.x, Rasgon PLoS One 2012 doi:10.1371/journal.pone.0005833) can locally spread cargo genes nearly to fixation if sufficient organisms (>30% of the local population) are released. “Threshold-dependent” gene drives such as those employing under-dominance (Curtis Nature 218:268-269 1968, Akbari and Hay Curr. Biol. 2013, Reeves et al PLoS One 2014) will spread to fixation in small and geographically isolated subpopulations if enough organisms are released to exceed the threshold (typically ˜50%) for population takeover. These prior containment methods are not sufficiently effective or workable for use in populations in the wild.

A solution that has been undertaken is to construct serially dependent CRISPR gene drive elements arranged in a daisy chain, which together form a “daisy drive” system (FIG. 8). They are arranged as a series of letters in the order opposite the alphabet, such that the terminal element carrying the “cargo” or “payload” is always “A”. Because the basal element in the chain (e.g. C in a three-element daisy drive system) does not exhibit drive, its abundance is typically limited to the initial frequency at which it is released in the population, modulated by the fitness cost of all the daisy drive elements to the organism. The next element exhibits drive only when the basal element is present, and so tends to lose the ability to exhibit drive swiftly (FIG. 9).

Recombination events between the elements of a linear daisy drive system have the potential to create a necklace of mutually dependent elements that can exhibit global drive (FIG. 5). Hence, homology between the elements must be minimized, which is accomplished using methods such as those set forth in: Example 1, Method 1.0 to identify highly divergent guide RNA sequences; Example 2, Method 2.3 to identify different promoters; and Method 3.1 to express multiple guide RNAs with minimal homology.

Models that were prepared predicted that daisy drives are more effective (e.g. they behave more like global self-sustaining drive systems) the more elements they have (FIG. 10). Constructing daisy drive organisms requires that one independent gene insertion event must occur for every element in the chain. Methods have been determined that can be used to generate daisy drive chains capable of different purposes, such as the alteration of distinct genes or of population suppression, using the same base chain. Specifically, methods have been developed for designing and constructing a daisy chain gene drive organism containing N−1 elements, where N is the total number of elements in the chain desired, is generated such that the terminal element in that chain (hereby designated the B element) encodes the RNA-guided DNA nuclease. Subsequently, a) a new A element accomplishing the desired change, be it alteration or suppression, and also encoding guide RNAs enabling it to drive in the presence of the RNA-guided DNA nuclease, is added to the organism's genome directly by standard methods known to those in the art so as to create a complete N-element daisy drive organism, or b) a new A element accomplishing the desired change, be it alteration or suppression, and also encoding guide RNAs enabling it to drive in the presence of the RNA-guided DNA nuclease, is separately inserted into the genome of another organism of the same species, which then is crossed with the daisy drive line in the laboratory so as to create a complete N-element daisy drive organism, or c) the (N−1) element organisms are released into the environment to initiate a daisy drive effect that spreads the gene encoding the RNA-guided DNA nuclease through the local population, after which organisms encoding any desired “A” element can be subsequently released to accomplish the desired effect, noting that while suppression will eliminate the RNA-guided DNA nuclease from the population, alteration can be accomplished multiple times in series or in parallel using effector A elements.

Method 3.0—Constructing a Daisy Drive Organism of (N−1) Elements that Lacks Only Element A.
(1) A sufficient number of sequence-divergent guide RNAs are identified (e.g. using Method 1.0) for the desired number of daisy drive elements. Each element except one is assumed to require multiple (at least 2, preferably more) guide RNAs. All guide RNAs encoded in the organism are sequence-divergent to minimize the potential for recombination between different elements.
(2) N−1 target genes and expression conditions for the RNA-guided DNA nuclease (e.g. using Example 2, Method 2.0) are identified. Methods below describe designing and constructing a four-element daisy chain gene drive, but the methods are also used to create longer and shorter daisy chain gene drives, that have three elements (a C-B-A daisy chain gene drive) or five, six, seven, eight, nine, ten, or more elements in longer daisy chain gene drives.
(3) The positions of the final daisy chain elements are defined in descending alphabetical order such that position A is the desired alteration, so, for example, a 4-element drive is D-C-B-A. In this case, this method involves creating D-C-B such that any A can be added. Element B is constructed first with the selection of a target gene and recoding the target gene sequence according to Method 3.3. A RNA-guided DNA nuclease is encoded downstream of the 3′UTR under appropriate expression conditions according to Example 2, Method 2.0.
(4) The C element is constructed next by encoding two or more guide RNAs recognizing the target gene of the B element just downstream of the C element target gene. The C element target gene is selected and recoded and its 3′UTR is replaced with one from another gene that has similar expression conditions (see Method 3.3). This is done in the strain containing B element or in a separate strain. The guide RNAs are designed and they are expressed using appropriate promoter(s) and processing methods—see Method 3.1. Also see Method 9.0 for an alternative way to encode guide RNAs in daisy drive elements.
(5) The elements for D, E, etc. are constructed as described for element B (step 4) until all the desired drive elements have been constructed. If the drive elements are constructed in separate strains, crosses are performed to combine all elements in a single strain, a process that can be assisted via the activity of the daisy drive. If a potential application for the prepared daisy chain gene drive may involve organism population suppression via a sex-specific effect, it can be advantageous to encode the highest/proximal element of the daisy chain (e.g. E in an E-D-C-B-A chain) within a locus exclusive to the unaffected sex.
(6) The resulting strain designed and constructed as in Method 3.0 exhibits daisy drive to spread element B, which encodes the RNA-guiding DNA-binding nuclease, through the local population.
(7) To add an element A encoding a desired effect, another strain is made that includes the desired genomic change and guide RNAs capable of driving that change by cutting the wild-type version. See Example 4, Method 4.0 for additional details regarding population suppression methods.
Method 3.1—Expressing Multiple Guide RNAs with Minimal Homology
(1) If testing or previous studies of RNA interference or CRISPR-mediated genome editing have identified polymerase III promoters capable of strong RNAi or guide RNA expression, those are used in the design and construction of daisy chain gene drives. Examples of suitable polymerase III promoters are for example: U6, H1, and tRNA promoters. If suitable promoters are not known, Example 2, Method 2.3 is used to identify promoters suitable for the type of daisy chain gene drive system that is designed and constructed. In some organisms, it may be possible to express guide RNAs from polymerase II promoters, sometimes using ribozymes or tRNAs for appropriate processing (see Method 3.2). Note that promoters cannot be re-used across daisy drive elements.
(2) It is possible to express two guide RNAs from a single polymerase III promoter by replacing the poly-T stretch leading to transcriptional termination of the first guide RNA with a 10-15 base pair linker to the second guide RNA. If earlier steps identify sufficient active promoters to express enough guide RNAs (2+ per element) for the desired daisy drive system, do so.
(3) To express more guide RNAs from a single promoter, a tRNA-based processing strategy is used. This approach also permits the guide RNAs to be processed to any desired length, potentially increasing specificity. See Method 3.2 to identify tRNAs suitable for processing.
Method 3.2—Identifying tRNAs Suitable for tRNA-Guide RNA-tRNA Array Processing
(1) A strain is constructed in which the RNA-guided DNA nuclease is expressed using a housekeeping gene enhancer/promoter/3′UTR such as actin that also expresses a fluorescent protein, either from a separate promoter or via 2A peptide fusion.
(2) Additional strains are constructed in which a promoter previously demonstrated to be effective in that organism (e.g. U6/H1/tRNA or one identified via Example 2, Method 2.3) drives a construct consisting of a tRNA, a control guide RNA that does not target any sequence in the cell, a different tRNA to be tested, a guide RNA targeting the gene encoding the fluorescent protein (or an equivalent recessive marker gene), a third tRNA, and another control guide RNA. The strains are crossed and fluorescence is measured in the progeny. Less fluorescence indicates more effective tRNA processing. The process is repeated, varying different tRNAs in the second and third positions, until sufficient tRNAs have been identified for processing of all daisy drive elements.
(3) Alternatively, the above experiment design and construction is performed in cultured cells. For example, the two DNA fragments described in the preceding paragraph are combined into one construct (which also encodes a different fluorescent protein as a marker of successful DNA delivery) and that DNA sequence is delivered into cultured cells of the target species. A standard method such as fluorescent-assisted cell sorting is used to isolate cells with the fluorescent marker that received the DNA. The cells are further sorted to identify cells that also lack the fluorescent gene targeted by the guide RNA, as these are cells in which tRNA-processing was effective in that it produced an active guide RNA that cut the fluorescent gene. The DNA is extracted and sequenced (in some instances using high-throughput) and tRNAs that worked are identified.
(4) Alternatively, a large library is prepared that includes DNA fragments encoding: (RNA-guided DNA nuclease, promoter-site1-tRNA1-(guide RNA targeting site 1)-tRNA2-(guide RNA targeting site 2)-tRNA3-(control guide RNA)-(site 2) for many different tRNAs of interest in different combinations. These DNA fragments are delivered into cells of the target species by standard methods. DNA is extracted from the cells, amplified using flanking primers to amplify site 1, site 2 and the region between, and then the amplicons are sequenced. The sequencing in some experiments may be high-throughput sequencing. Any sequence reads with clear mutations in site 1 or site 2 indicate correct processing activity by the flanking tRNAs.

### Method 3.3—Recoding a Target Gene for the Purposes of Building a Gene Drive

(1) Studies are performed that identify suitable target sites with few off-targets within the coding sequence of the gene. The coding sequence of the gene are recoded from the target sites to the end of the coding sequence by changing every codon if possible, removing all introns in between, and replacing its 3′UTR with one from another gene with similar expression conditions.
(2) Either guide RNAs (as in Method 3.1) or an RNA-guided DNA nuclease or both are encoded downstream of the 3′UTR as needed for the particular application, but there must be no homology between 3′UTR and any such inserted elements.

### Example 4

Provided are Methods of Building and Using Serially Dependent 1-Dimensional Daisy Chains of Gene Drive Elements (Daisy Drive) Organisms Wherein the Terminal Element that Exhibits Drive Results in Population Suppression Through Either Sex-Biasing (Via Targeting a Sex Chromosome in the Germline after the Soma-Germline Division has been Specified but Before Meiosis Such that Surviving Gametes Will Produce Individuals Mostly of One Sex) or Genetic Load, (Via Disrupting Genes Essential for Viability or Fertility in One or Both Sexes in the Germline after the Soma-Germline Division has been Specified but Before Meiosis).

Methods of suppressing populations using endonuclease gene drive elements to bias the sex ratio or impose a genetic load have been previously described (Burt, A. 2003 Proc. Roy. Soc. Lond. B. 270,921-8; and Esvelt et al 2014 eLife:e03401, the content of each of which is incorporated herein by reference in its entirety). However, such gene drive elements are inherently self-sustaining and consequently pose risks to all populations of the target species anywhere in the world. New methods are provided herein that are used to limit population suppression to local rather than global populations by creating “daisy chain” gene drive elements that cause local population suppression.

Specifically, methods are provided for the design and construction of a daisy drive chain of any length can be constructed in which the each element requires the prior link in order to drive, and the first element in the chain does not exhibit drive. By including a terminal element at position A that imposes genetic load (FIG. 14) or generates a sex-biasing effect, the daisy drive element suppressed the population in the area of release, but because it is a limited daisy drive rather than a self-sustaining drive, that effect will be limited to the area of release.

Any potential configuration of daisy drive elements can be adjusted using methods provided herein to induce a population suppression effect. For example, a daisy chain gene drive can be designed and constructed in which target element A can replace and therefore eliminate a recessive gene that is important for viability or fertility as would a self-sustaining/global genetic load drive, or a daisy chain gene drive may be designed and constructed that includes multiple guide RNAs that target and disrupt such a gene. Alternatively, element A could be a standard daisy drive element (as described in Example 3, Method 3.0) that encodes both guide RNAs targeting such loci for disruption as well as guide RNAs causing itself to drive. Alternatively, it could include an extra copy of the single gene or set of genes that ensure the organism will develop a one particular sex in the relevant specie; for example, a single copy of the Sry gene in mice causes maleness. Alternatively, the A element could include guide RNAs inducing the RNA-guided DNA nuclease to cut and eliminate a sex chromosome, thereby ensuring that nearly all offspring of A element organisms are of one sex. These and other strategies for population suppression can be utilized in methods to design and construct daisy chain gene drives.

### Method 4.0—Suppressing a Population Via Genetic Load

(1) Recessive genes are identified that correspond to, in order of preference, sex-specific infertility, infertility, sex-specific viability, or viability by combing the literature or standard genetic techniques.
(2) In the wild-type background, a strain is designed and constructed in which a large portion or all of such a gene is replaced by guide RNAs targeting the wild-type version, such that the guide RNAs exhibit drive in the presence of the RNA-guided DNA nuclease.
(3) Alternatively, a new daisy drive element (per Example 3, Method 3.3) is designed and constructed via genetic recoding, but one that expresses both guide RNAs that will allow it to exhibit drive by targeting the wild-type version of its own locus in the presence of the RNA-guided DNA nuclease and also guide RNAs leading to disruption of one or more genes identified in Step 1. It is possible to target several such genes in the same organism for increased evolutionary robustness.
(4) The resulting strain is crossed with a daisy drive strain created via Example 3, Method 3.0 to create a complete daisy drive strain. Homozygose and employ methods of inhibiting suppression activity in the production facility (Example 3, Method 3.3) as needed. If the suppression method is sex-specific, it is best to use a daisy drive strain in which the proximal element is located within a locus exclusive to the unaffected sex. For example, in Aedes aegypti a daisy drive system disrupting female fertility genes should have the proximal element in the daisy drive chain located within the M locus exclusive to males, thereby ensuring that males carrying the complete drive system are unaffected by suppression, save through mating with sterile females.
(5) Alternatively, add guide RNAs targeting genes identified in Step 1 to element B of the daisy drive strain created via Example 3, Method 3.0. This element is now element A, and other elements are redesignated accordingly.
(6) Information is obtained to determine how many organisms must be released to suppress a target population of a given size to the desired level. The information in some instances may be obtained using cage studies and field trials.
(7) The target population is sampled and the number of organisms required for release is estimated. Based on the estimate, a suitable number of daisy drive organism are released into the target environment to suppress or eliminate the target species from the local area.

### Method 4.1—Suppressing a Population by Causing Drive-Carrying Organisms to Develop as a Single Sex

(1) Identify a gene whose presence or disruption causes individuals to develop as a particular sex through standard genetic methods (e.g. Sry causes maleness in Mus musculus and Nix in Aedes aegypti, while the loss of fem-3 causes femaleness in C. elegans).
(2) A transgenic organism is created with a new daisy drive element (per Example 3, Method 3.3) via genetic recoding, but one that expresses both guide RNAs that will allow it to exhibit drive by targeting the wild-type version of its own locus in the presence of the RNA-guided DNA nuclease and also guide RNAs leading to disruption of one or more genes identified above. It is possible to target several such genes in the same organism for increased evolutionary robustness.
(3) Alternatively, a transgenic organism is created with a new daisy drive element (per Example 3, Method 3.3) via genetic recoding, but one that expresses guide RNAs that will allow it to exhibit drive by targeting the wild-type version of its own locus in the presence of the RNA-guided DNA nuclease and also a gene identified above, in Step 1 causing development as a single sex. It is possible to create several such elements in the same organism for increased evolutionary robustness. Methods of inhibiting sex-biasing activity in the organism production facility are employed (Example 3, Method 3.3 or using a tet-OFF system to control expression of genes causing development as one particular sex) as needed.
(4) The resulting strain is crossed with a daisy drive strain created via Example 3, Method 3.0 to create a complete daisy drive strain. Homozygose.
(5) Alternatively, guide RNAs targeting genes identified in Step 1 are added to element B of the daisy drive strain created via Example 3, Method 3.0. This element is now element A and other elements are redesignated accordingly.
(6) A determination is made as to how many organisms must be released to suppress a target population of a given size to the desired level using cage studies and field trials.
(7) The target population is sampled to estimate the number of organisms required for release based on the determination. A suitable number of daisy drive organism are released into the target environment to suppress or eliminate the target species from the local area.

### Method 4.2—Suppressing a Population Via Chromosomal Shredding

(1) A set of sequences are identified on either side of the centromere of the sex chromosome that corresponds to the sex a population will be biased against (e.g. the X to male-bias in mice). An off-target-finding software (e.g. sgRNACas9 or GT-Scan) is used as normal to ensure the sites are sufficiently unique in the genome. Ideally, the identified target sequences are unique to the target chromosome but are repeated several times.
(2) In an (N−1) daisy drive (e.g. D-C-B) background, a new target gene (not yet used in the daisy drive) is identified for the daisy drive element A following Example 2, Method 2.0.
(3) The gene is recoded following procedure of Example 3, Method 3.3 and at least two guide RNAs are encoded that target the wild-type version of the target gene and that function with the RNA-guided DNA nuclease encoded in the B element of the daisy drive strain.
(4) An orthogonal RNA-guided DNA nuclease is also encoded such that it is expressed exclusively during late meiosis (see Windbichler N. et al 2011 Nature 473, 212-215; and Port, F. et al., 2014, PNAS vol. 111 no. 29; E2967-E2976, each of which is incorporated by reference herein in its entirety). Guide RNAs for this nuclease are also encoded that target the sequences identified in step 1. The progeny ratio should be biased towards the desired sex; adjust expression conditions until this occurs.
(5) If the proximal element of the daisy drive chain is not encoded within the sex-determining locus or chromosome favored by the A element, the daisy drive strain is simply crossed to wild-type organisms of the non-favored sex to maintain the population and produce organisms for release. See Example 6, Method 6.0 for additional details.
(6) If the proximal element of the daisy drive chain is not encoded within the sex-determining locus or chromosome favored by the A element, a strain is generated that contains only the proximal element of the daisy drive system (e.g. element D for a D-C-B-A system). The daisy drive organisms are crossed to this strain (sorted for the non-favored sex) to maintain the population and produce organisms for release.
(7) A determination is made of the number of gene drive organisms that must be released to suppress a target population of a given size to the desired level using cage studies and field trials.
(8) The target population is sampled to estimate the number of organisms required for release based. A suitable number of daisy drive organism are released into the target environment to suppress or eliminate the target species from the local area.

### Example 5

Methods of Building and Using Serially Dependent 1-Dimensional Daisy Chains of Gene Drive Elements (Daisy Drive) Organisms with an Arbitrary Number of Elements Such that the Terminal Element Targets and Recodes a Gene Important for Organismal Fitness as it Spreads in Order to Enable the Subsequent Alteration or Suppression of Exclusively the Previously Altered Local Population at a Later Date.

Altering a population with a daisy drive permits subsequent precision targeting of the introduced sequence with a global CRISPR gene drive system, which will not spread beyond the target population. This is a “precision drive” strategy. It is most effective if the terminal element of the daisy drive alters a gene suitable for targeting with a suppression drive.

### Method 5.0—Two-Stage Suppression Using Guide RNAs Only.

1. Recessive genes are identified that corresponding to, in order of preference, sex-specific infertility, infertility, sex-specific viability, or viability by combing the literature or standard genetic techniques.
2. In a wild-type background, one or more of the target genes is replaced with guide RNAs targeting sites within the replaced sequence. Guide RNAs are encoded using expression conditions determined using Example 3, Method 3.1.
3. Laboratory cage studies and/or field trials are performed to determine how many organisms of this strain must be released to suppress or eliminate a population of organisms that already encode an RNA-guided DNA nuclease.
4. It is determined how many organisms of an already-available daisy drive system, such as one created via Example 3, Method 3.0 that has an RNA-guided DNA nuclease as its terminal element, must be released to drive the nuclease gene to fixation in the population to be suppressed or eliminated.
5. Daisy drive organisms are released in suitable numbers to accomplish local fixation. The population into which the daisy drive organisms were released is sampled to ensure the daisy drive has spread to the desired extent.
6. The strain created in step 2 is released in sufficient numbers to suppress or eliminate the population as desired.
7. Alternatively, the population is suppressed by biasing it towards one sex. A suitable target gene or genes are identified using Example 2, Method 2.0. In a wild-type background, the gene(s) are recoded using Example 3, Method 3.3. Just downstream of the new 3′UTR of the gene(s), guide RNAs are encoded that correspond to target sites within the wild-type version of the gene so that it can drive itself in the presence of the appropriate RNA-guided DNA nuclease. A gene is included that ensures carrier organisms develop as a particular sex, or encode guide RNAs that disrupt a gene causing the same outcome, or target sites are identified for chromosomal shredding as in Example 4, Method 4.2, step 1 and an orthogonal RNA-guided DNA nuclease is encoded such that it is expressed exclusively during late meiosis (see Windbichler et al Nature 2011, Port et al PNAS 2014) as well as guide RNAs for this nuclease that target the sequences causing chromosomal shredding. Refer to Example 4, Methods 4.0, 4.1, and 4.2 for additional details on suppression mechanisms.

### Method 5.1—Two-Stage Suppression Using Genetic Load

(1) Recessive genes are identified that correspond to, in order of preference, sex-specific infertility, infertility, sex-specific viability, or viability by combing the literature or standard genetic techniques.
(2) One or more of these genes is recoded via Example 3, Method 3.3, ensuring that the recoded sequence contains multiple suitable target sites for a subsequent gene drive system with few or no off-targets in the genome.
(3) Just downstream of the 3′UTR of the gene or genes, guide RNAs are encoded that correspond to target sites within the wild-type version of the gene such that the element drives itself in the presence of an RNA-guided DNA nuclease.
(4) The resulting strain(s) are crossed with a daisy drive strain created via Example 3, Method 3.0 to create a complete daisy drive strain that recodes the target gene(s). Homozygose.
(5) In a wild-type background, one or more of the target genes is replaced with an RNA-guided DNA nuclease encoded using expression conditions determined in Example 2, Method 2.0, and also guide RNAs targeting sites within the first recoded version of the gene, which are encoded using expression conditions determined using Example 3, Method 3.1.
(6) The target population is sampled to estimate the number of organisms. A suitable number of daisy drive organisms created in Step 4 are released in the target environment to recode the nearby population.
(7) Organisms are sampled and sequenced (or checked for a marker gene inserted into the A element of the daisy drive) to verify that a suitable fraction of the relevant population has been recoded. In most cases this entails fixation in the target local population.
(8) Organisms carrying the suppression drive(s) generated in step 5 are released into the target environment. The drive(s) spreads through and suppresses the population recoded with the daisy drive, but not wild-type organisms.

### Method 5.2—Two-Stage Suppression Using Sex-Biasing or Sex Chromosomal Shredding

(1) A suitable target gene or genes is identified using Example 2, Method 2.0.
(2) In a wild-type background, the gene(s) are recoded via Example 3, Method 3.3, ensuring that the recoded sequence contains multiple suitable target sites for a subsequent gene drive system with few or no off-targets in the genome.
(3) Just downstream of the new 3′UTR of the gene(s), guide RNAs corresponding to target sites within the wild-type version of the gene are encoded such that the element drives itself in the presence of an RNA-guided DNA nuclease.
(4) The resulting strain(s) are crossed with a daisy drive strain created via Method 3.0 to create a complete daisy drive strain that recodes the target gene(s). Homozygose.
(5) A suitable target gene or genes is identified using Example 2, Method 2.0. In a wild-type background, the gene(s) are recoded using Example 3, Method 3.3. Just downstream of the new 3′UTR of the gene(s), guide RNAs corresponding to target sites within the wild-type version of the gene are encoded so that it can drive itself in the presence of the appropriate RNA-guided DNA nuclease. A gene is included that ensures carrier organisms develop as a particular sex, or guide RNAs are encoded that disrupt a gene causing the same outcome, or target sites are identified for chromosomal shredding as in Example 4, Method 4.2, step 1 and an orthogonal RNA-guided DNA nuclease is encoded such that it is expressed exclusively during late meiosis (see Windbichler et al Nature 2011, Port et al PNAS 2014) as well as guide RNAs for this nuclease that target sequences causing chromosomal shredding.
(6) The target population is sampled and the number of organisms is estimated. A suitable number of daisy drive organisms created in Step 4 are released in the target environment to recode the nearby population.
(7) Sample organisms are sampled and sequenced (or check for a marker gene inserted into the A element of the daisy drive) and it is verified that a suitable fraction of the relevant population has been recoded. In most cases this entails fixation in the target local population.
(8) Organisms carrying the suppression drive(s) generated in step 5 are released into the target environment. The drive(s) spread through and suppress the population recoded with the daisy drive, but not wild-type organisms.

### Example 6

### Methods of Achieving Stable Population Suppression by Locating the First Element in the Daisy Drive Chain in a Position Unique to One Sex and Suppressing Fertility or Viability of the Other Sex.

Daisy drive systems used directly for population suppression will experience a fitness cost limiting their potency. It is possible to ensure that the incidence of the daisy drive remains nearly proportional to the current population by reducing the fertility or viability of one sex while locating the first element of the daisy chain adjacent to a gene unique to the other sex.

For example, a simple C→B→A daisy drive might encode the guide RNAs of the C element adjacent to a male-determining gene (e.g. the Nix gene within the M factor of the dengue vector Aedes aegypti) or a sex chromosome unique to males (e.g. the Y chromosome in the malaria vector Anopheles gambiae). The RNA-guided DNA nuclease is encoded at a B element as is standard for a daisy drive. The A element would include guide RNAs that target and either disrupt or replace female fertility or viability genes. Alternatively, guide RNAs disrupting these genes might be encoded on the B element leaving the A element without guide RNAs of its own.

As a result, daisy drive males would inactivate the female fertility genes during gametogenesis. Their sons would always inherit the C element (as well as B and A thanks to drive) and would suffer minimal fitness penalty, allowing them to repeat the cycle as it occurred in their fathers. Daughters would inherit one copy of the B element and the A element. During gametogenesis, the A element would drive thanks to the presence of the B element, so all offspring of these daughters would inherit a broken copy. If the other parent is a daisy drive male, their daughters will be sterile, thereby suppressing the population.

Method 6.0—Building a Daisy Drive System for Population Suppression with Reduced Fitness Cost
(1) Steps 1-5 of Example 3, Method 3.0 are followed to generate a basic daisy drive. Wild-type sequences within the proximal element (e.g. element D if it is a D-C-B drive system) are noted.
(2) A genetic element is identified that is specific to the sex that will NOT be targeted by the drive system (e.g. if the drive system disrupts female fertility, an element specific to males).
(3) In the daisy drive strain, guide RNAs are encoded that target the wild-type version of the currently proximal element in the daisy drive chain within or adjacent to the sex-specific genetic element. Guide RNAs are encoded according to Example 3, Method 3.1.

### Example 7

### Methods of Achieving Stable Population Suppression by Using a Daisy Intermediate to Inactivate Female Fertility Genes in a Dominant Manner.

A variation on the above Examples involves ensuring that the A element exhibits drive in the zygote, thereby ensuring that any female inheriting a single copy of the B element is sterile (or nonviable). This is achieved by arranging for the RNA-guided DNA nuclease encoded in B to be expressed in the zygote and/or the early stages of development. This will cause it to disrupt the wild-type allele of the A element inherited from the other parent, resulting in sterile or nonviable females. Because the fitness cost to males will be minimal, the introduction of males of this type will cause immediate population suppression proportional to the fraction of daisy drive males. This approach may be necessary because there are few genes whose loss causes dominant sterility in a sex.

Daisy chain drive systems designed in this manner permit controlled and persistent population suppression by linking a sex-specific effect to a genetic locus unique to the other sex. For example, female fertility genes such as those recently identified in malarial mosquitoes {Hammond et al 2015 Nat. Biotech.} are targeted by a genetic load daisy drive whose basal element is located on the Y chromosome or an equivalent male-specific locus (FIG. 14). These males suffer no fitness cost due to suppression relative competing wild-type males. The 3-element daisy chain drive system in which female fertility gene disruption occurs early in development creates a male-linked dominant sterile-daughter effect that is otherwise very difficult to generate genetically. By enabling local population levels to be titrated in a controlled and reversible manner, daisy chain drives prove a valuable tool for the study of ecological interactions.

### Method 7.0—Building a Sex-Linked Drive System Causing Dominant Sterility in Opposite-Sex Offspring.

(1) Steps 1-3 of Example 3, Method 3.0 are followed to generate a strain with just the B element of a daisy drive system, except that the RNA-guided DNA nuclease must be encoded such that active nuclease will be present in the zygote and early embryo (e.g. employ a constitutive or housekeeping promoter such as the actin promoter).
(2) A genetic element is identified that is specific to the sex that will NOT be targeted by the drive system (e.g. if the drive system disrupts female fertility, an element specific to males).
(3) In the strain with element B, guide RNAs are encoded that target the wild-type version of element B within or adjacent to the sex-specific genetic element. Guide RNAs are encoded according to Example 3, Method 3.1. This is element C, which will cause element B to drive in organisms of the appropriate sex.
(4) Steps 1-3 of Example 4, Method 4.0 are followed, being sure to use genes corresponding to sex-specific infertility.
(5) The resulting strain is crossed to the (C-B) daisy drive strain to create a sex-specific daisy drive strain whose opposite-sex offspring are infertile due to loss of both copies of the target gene(s). Same-sex offspring are (C-B-A) daisy drive organisms of nearly normal fitness.
(6) If the above method results in a drive with low homing efficiency and consequent loss of offspring due to non-homologous end-joining in the B target gene, the sex-specific C element is constructed such that it encodes its own orthogonal RNA-guided DNA nuclease expressed in the germline just after the soma-germline division per Example 3, Method 3.0, as well as guide RNAs directing it to cut the wild-type target gene in element B.

### Example 8

Methods of Designing and Constructing Daisy Drive Elements in which Guide RNAs are Embedded within Introns of Target Genes.

Some genes may not be amenable to recoding at the 3′ end, or to having their 3′UTR replaced. An alternative method has been developed in which the guide RNAs are encoded within the gene itself. This is most effective when the gene is highly transcribed; fortunately, most haploinsufficient genes chosen as daisy drive targets are ribosomal and are consequently some of the most highly expressed in the cell. However, guide RNAs must be produced from these transcripts without disrupting the function of the gene. A solution has been developed that includes embedding the guide RNAs within introns, separated by tRNAs for efficient processing. The tRNA-processing method has been shown to enable high nuclease activity in fruit flies when driven by strong polymerase II promoters (http://dx.doi.org/10.1101/046417); ribozyme-based processing (not suitable for daisy drive due to repetitiveness) works efficiently from within introns (http://dx.doi.org/10.1016/j.molce1.2014.04.022). To ensure that the guide RNAs are copied efficiently, the target wild-type gene must be cleaved on both sides of the intron.

Method 8.0—Embedding Daisy Drive Elements within Introns of Target Genes
(1) For daisy drive elements that would otherwise encode guide RNAs encoded downstream of recoded and 3′UTR-swapped highly-expressed target genes, an alternative design is used that includes inserting a string of alternating tRNAs and guide RNAs into an intron such that tRNAs are on either flank of the string. Example 3, Method 3.2 is used to identify tRNAs suitable for processing in the relevant organism. It may be necessary to attempt insertion at several such sites in case insertion at one site disrupts splicing; this is less of a risk for larger introns which should be preferred targets. Disrupted splicing is manifested as inviability or extremely poor growth because the target gene should be haploinsufficient.
(2) Nuclease target sites are recoded in the exons on either side of the intron. To reduce the risk of creating a drive-resistant allele through multiple short homology-directed repair events, at least two target sites are included on either side. Optionally, the sequence is recoded between the sites closest to the intron and the boundaries of the intron itself, while leaving the 6-12 bp closest to the splice junction unaffected to minimize the risk of disrupting splicing.
(3) The guide RNAs in the upstream element of the daisy chain should target the recoded sites.

### Example 9

### Methods for Building Evolutionarily Unstable Yet Robust Drive Systems Through Redundancy.

Homing-based gene drive systems are vulnerable to drive-resistant alleles that block drive copying and thus prevent the spread of the drive system. These alleles are generated naturally whenever the endonuclease cut is repaired by non-homologous end-joining, which can create indels or point mutations at the target site that block subsequent cutting. This is why evolutionarily stable drives must target multiple sites within genes important for fitness. However, it is possible to affect large numbers of organism even without evolutionary stability. A typical rate of NHEJ repair is 5% (Gantz, V. & Bier, E. 2015 Science 24 April: Vol. 348, Issue 6233, pp. 442-444; Gantz, V. et al., 2015 PNAS Vol. 112 no. 49 E6736-E6743; and Hammond, A. et al., Nat Biotechnol. 2015 Dec. 7; doi:10.1038/nbt.3439, the contents of each of which is incorporated herein by reference in its entirety). Thus, at minimum 5% of the population will be unaffected by the drive system; the share will decline as natural selection favors the resistant alleles over the drive. This precludes suppression drive strategies, but may be acceptable for certain alteration-based requirements. One method of compensating is to build multiple evolutionarily unstable drive systems, each of which targets a single site. There is no need to target a sequence important for fitness as there is no need to select against drive-resistant alleles.

Similar logic has now been applied to daisy drive systems. Because a daisy drive system is not intended to spread indefinitely, each element will only be copied a fixed number of times. This limits the potential for drive-resistant alleles to emerge that block spread. However, this is counterbalanced by the increased number of elements that must be copied, which increases vulnerability to any one drive-resistant allele.

Method 9.0—Building Redundant Evolutionarily Unstable Drive Systems that Target the Same Locus
(1) Instead of building a single homing-based drive system based on an RNA-guided DNA nuclease with multiple guide RNAs, multiple drive systems are built, each of which targets a different sequence or sequences within the same locus. Multiple target sites within a given locus (at least two, preferably more) are identified.
(2) A drive system is constructed by encoding an RNA-guided DNA nuclease with appropriate expression conditions for comparatively efficient homologous recombination as opposed to NHEJ, such as is determined by Example 2, Method 2.0. However, any expression conditions acting upon cells that will eventually compose the germline will do. Additionally, a single highly active promoter is encoded (e.g. identified using Example 2, Method 2.3) that drives a guide RNA targeting one of the target sites. This inserted DNA replaces all target sites identified within the locus.
(3) Additional drive systems are constructed that target all of the other target sites within the locus. Drive systems will not be able to cut and replace one another and so will coexist within the target cell. A drive-resistant allele for one system will not resist another system. Only the gradual accumulation of drive-resistant alleles at all sites will fully block the spread of the drive system. This will eventually occur if the population to be altered is sufficiently large, but it may not matter for many applications.
(4) Organisms comprising all drive systems together are released.
(5) To build equivalent daisy drive systems, this strategy is repeated for each daisy drive element. That is, build multiple daisy drive systems, each of which has one guide RNA per element that targets a different site within the wild-type locus harboring the next element in the chain (or in the case of A elements that encode their own guide RNAs, their own locus).

### Example 10

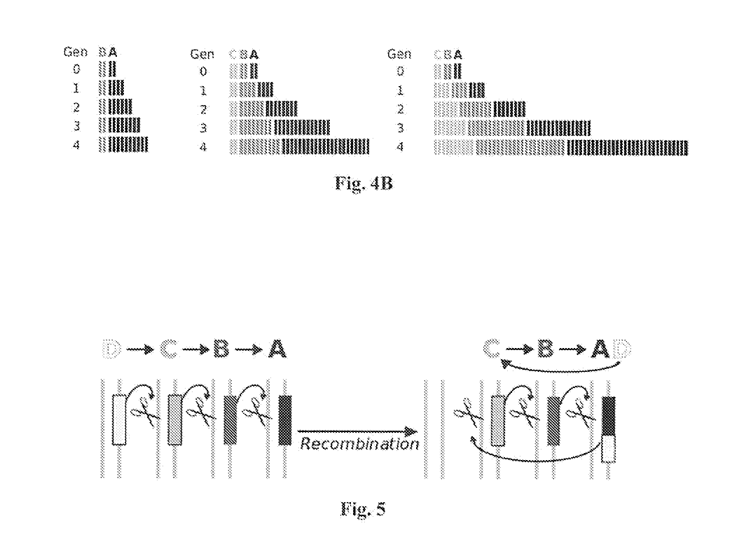

Family tree analysis was performed and results indicated the power of including additional elements to prepare a daisy chain gene drive. Results are shown in FIGS. 4&5. A simple deterministic discrete-generation model of allele frequencies in an isolated panmictic population was prepared and used to analyze the likely effects of seeding at an arbitrary frequency. Each element was assigned a dominant multiplicative fitness cost to account for imperfect homing, gene expression, any off-target cutting, and other losses. The model assumed that each active element was designed to prevent the evolution of drive resistance alleles as previously described (Esvelt et al. 2014 eLife e03401, the contents of which is incorporated herein by reference in its entirety).

Results of the modelling indicated that a C→B→A daisy drives will spread A to near-fixation when released at low but not very low frequencies (FIG. 11A-B). However, the drives were highly sensitive to the fitness costs incurred by elements B and C (FIG. 11C). FIG. 11A shows that a daisy drive with 2% fitness cost per upstream element and 10% fitness cost for the final element, seeded at 1%, never approaches fixation. FIG. 11B shows that the same drive seeded at 5% would rapidly fix in a non-deterministic model. FIG. 11C shows that if the upstream elements cost 10% each, more organisms would need to be released.

It was determined that adding elements to the daisy chain should help compensate for higher fitness costs, and a formal model was constructed and used to evaluate the consequences of adding additional elements. It was observed that longer daisy chains lead to much stronger local drive (FIG. 12). At a cost of 5% per daisy drive element, which is readily accessible to current drive systems, four- and five-element daisy drive systems driving a payload with 10% cost could be released at frequencies as low as 2.5% and 1% respectively and still exceed 99% frequency in less than 20 generations without global spread. FIG. 12 illustrates the finding that the A element attains higher frequencies as daisy-chain length increases across a range of fitness costs per upstream element, assuming the final element has a fitness cost of 10%. FIG. 12A shows that with population seeding at 5%, three element chains are sufficient for the A element to reach 99% frequency if the upstream elements have a low fitness cost (2%, left). As the cost increases to 5% (middle), four elements are required, and 10% cost precludes spread above roughly 80%. FIG. 12B shows results that indicated that daisy drives with more elements require fewer organisms to be released in order for the A element to reach a frequency of 99%. Each homing event was assumed to occur with 95% efficiency.

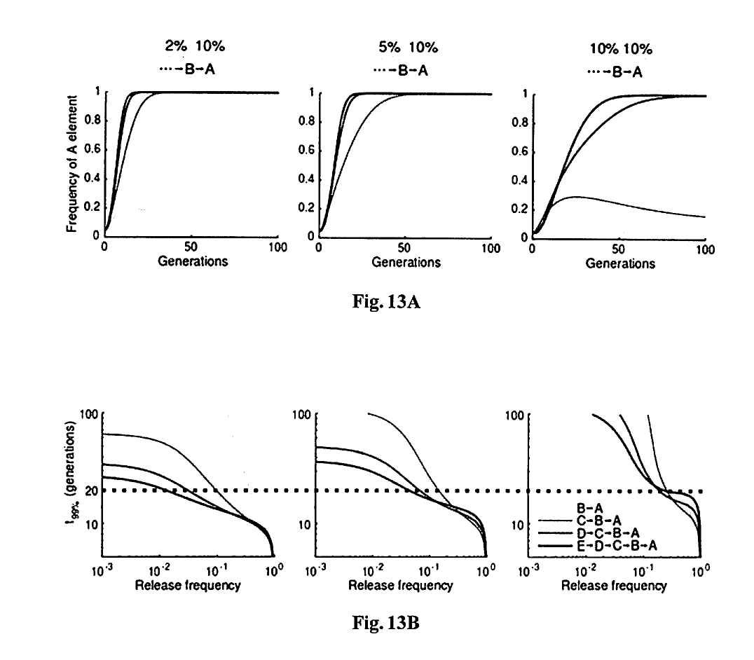

It was determined that for some applications, a periodic release of daisy chain gene drive organisms could provide more cost-effective population control than a single release of the daisy chain gene drive organisms. The model was adjusted to include additional releases in every subsequent generation and analysis of the modelling results indicated that daisy drives can readily alter local populations if repeatedly released in very small numbers (FIG. 13). It was determined that this method of daisy chain drive implementation could be used in applications that must affect large geographic regions over extended periods of time, as for local organism population eradication campaigns. FIG. 13A-B provides graphs illustrating that releasing new organisms in each generation enables faster spread and requires fewer organisms per release. The numerical simulations depicted in FIG. 13A-B are identical to FIG. 11, except the initial release is repeated each generation. The final construct is assumed to have a 10% fitness cost. FIG. 13A shows that three- four- or five-element daisy drives can spread constructs with upstream elements having fitness costs of 2% (left) or 5% (middle) to 99% frequency. Four- or five-element drives are sufficient when the upstream elements have higher (10%) fitness costs. FIG. 13B indicates that repeated release at very low frequency (0.1%) is sufficient for spread of the final element to 99% frequency for upstream elements having fitness costs of 2% (left) or 5% (middle), while >1% repeated release is required for higher cost (10%) elements.

### Example 11

It was determined that any recombination event that moved one or more guide RNAs within an upstream element of the chain into any downstream element would convert a linear daisy drive chain into a self-sustaining CRISPR gene drive ‘necklace’ (FIG. 5). Methods to reduce the likelihood of such recombination event were developed and tested.

It was determined that a way to reliably prevent such recombination events was to eliminate regions of homology between the elements. Means to remove promoter homology were developed that included use of different U6, H1, or tRNA promoters for each element {see Port et al. (2014) PNAS, Ranganathan et al (2014) Nat. Comm. doi:10.1038/ncomms5516, Mefferd et al (2015) RNA doi:10.1261/rna.051631.115}, by expressing multiple guide RNAs from a single promoter using tRNA processing {see Xie et al. (2015) PNAS doi:10.1073/pnas.1420294112, Port and Bullock (2016) bioRxiv doi:10.1101/046417} or by connecting a pair of sgRNAs by a short linker. However, it was recognized that each element still required guide RNAs that were over 80 base pairs in length, which precluded safe and stable daisy drive designs.

### Methods

Guide RNA Design: S. pyogenes Cas9

Examination was made of existing data on guide RNA variants and corresponding activities as well as the crystal structure of S. pyogenes Cas9 in complex with sgRNA to identify bases that would likely tolerate mutation. Using this information, a set of 20 sgRNAs were constructed and assays for activity (see below) using only two replicates to identify sequence changes that were harmful to activity. These experiments indicated that the large insertion found in sgRNAs from closely related bacteria was well-tolerated in only one case. The insertion was removed and additional sgRNAs designed. All of the designed sgRNA candidates were constructed and assayed to identify those with sufficiently high activity. For experiments requiring additional highly divergent sgRNAs, such as daisy suppression drives in which the “A” element encodes many guide RNAs that disrupt multiple recessive fertility genes at multiple sites, a more comprehensive approach to activity profiling is performed that examines additional candidate guide RNAs. FIG. 6 shows the sequences of candidate guide RNAs that were designed, constructed, and tested for activity (SEQ ID NOs: 3-35).

Measuring Guide RNA Activity: S. pyogenes Cas9

HEK293T cells were grown in Dulbecco's Modified Eagle Medium (Life Technologies) fortified with 10% FBS (Life Technologies) and Penicillin/Streptomycin (Life Technologies). Cells were incubated at a constant temperature of 37° C. with 5% CO₂. In preparation for transfection, cells were split into 24-well plates, divided into approximately 50,000 cells per well. Cells were transfected using 2 μl of Lipofectamine 2000 (Life Technologies) with 200 ng of dCas9 activator plasmid, 25 ng of guide RNA plasmid, 60 ng of reporter plasmid and 25 ng of EBFP2 expressing plasmid.

Fluorescent transcriptional activation reporter assays were performed using a modified version of addgene plasmid #47320, a reporter expressing a tdTomato fluorescent protein adapted to contain an additional gRNA binding site 100 bp upstream of the original site. gRNAs were co-transfected with reporter, dCas9-VPR, a tripartite transcriptional activator fused to the C-terminus of nuclease-null Streptococcus pyogenes Cas9, and an EBFP2 expressing control plasmid into HEK293T cells. 48 hours post-transfection, cells were analyzed by flow cytometry. In order to exclusively analyze transfected cells, cells with less than 10³ EBFP2 expression were ignored. The preliminary screen of the initial 20 designs was performed with only two replicates to identify critical bases. Experiments evaluating the final set of sgRNA sequences were performed with six biological replicates.

### Guide RNA Design: Acidaminococcus Cpf1

Examination was made of existing data on the crRNAs of various Cpf1 relatives to identify bases that would likely tolerate mutations. Using this information, variants with single mutations in the four bases preceding the stem or within the loop were constructed. Similarly, mutants that changed both paired bases at a position in the stem were constructed. In some variants, these mutations were combined. Variant repeats were inserted into arrays of ten crRNAs, each paired with a spacer sequence with a matching protospacer in a unique target plasmid. Repeat variants were located in different positions and paired with different spacers to control for position and spacer effects.

### Measuring Guide RNA Activity: Acidaminococcus Cpf1

Plasmid exclusion assays were performed by transforming cells expressing AsCpf1 and an array or control cells lacking an array with the target plasmids, plating, and measuring the difference in the number of colonies. The effectiveness of each variant was recorded for different positions and spacers consistently active, defined as exhibiting >10-fold exclusion in all cases, identified.

### Results

To identify highly active guide RNA sequences with minimal homology to one another, known tracrRNA, crRNA, and alternative sgRNA sequences for CRISPR systems related to that of S. pyogenes were compared and variable regions were identified. Dozens of sgRNA variants that had been designed to be as divergent from one another as possible were created. These candidate sgRNAs were assayed using a sensitive tdTomato-based transcriptional activation reporter identified 15 different sgRNAs with activities comparable to the standard version (FIGS. 6&13). This set of minimally homologous sgRNAs should enable stable daisy drive systems of up to 5 elements with 4 sgRNAs per driving element. Future studies will need to examine the stability of the resulting daisy drive in an animal model.

These divergent guide RNAs will also enable global CRISPR gene drive elements to overcome the problem of ‘drive-resistant alleles’ that cannot be cut and replaced. Targeting multiple adjacent sequences within genes important for fitness was previously described as a solution for this problem {Esvelt et al (2014) eLife}, but repetitive elements even within a single drive construct often prove unstable {Simoni et al (2014) Nucl. Acids Res.}.

### Example 12

### Guide RNA Activity Measurement Using a Transcriptional Activation Reporter Assay

The activity of candidate guide RNAs was measured and determined using a transcriptional activation reporter using dCas9-VPR.

(1) Mammalian cells were grown using standard conditions (e.g. HEK293T cells were grown in Dulbecco's Modified Eagle Medium (Life Technologies) fortified with 10% FBS (Life Technologies) and Penicillin/Streptomycin (Life Technologies), incubated at a constant temperature of 37° C. with 5% CO₂).
(2) The cells were split into 24-well plates, divided into approximately 50,000 cells per well and then transfected with plasmids encoding:

(a) dCas9-VPR (or the equivalent dead-nuclease transcriptional activator variant of the RNA-guided DNA-binding protein nuclease matching the guide RNAs to be tested),

(b) the guide RNA to be evaluated,

(c) a reporter plasmid comprising a minimal promoter and one or more protospacer binding site upstream of a gene encoding a fluorescent protein, and

(d) a control plasmid expressing a different fluorescent marker gene as a transfection control marker.

(3) The transfections were carried out as follows: using 2 μl of Lipofectamine 2000 (Life Technologies) with 200 ng of dCas9 activator plasmid, 25 ng of guide RNA plasmid, 60 ng of reporter plasmid and 25 ng of EBFP2 expressing plasmid. The reporter plasmid was a modified version of addgene plasmid #47320, a reporter expressing a tdTomato fluorescent protein adapted to contain an additional gRNA binding site 100 bp upstream of the original site, the activator is da tripartite transcriptional activator fused to the C-terminus of nuclease-null Streptococcus pyogenes Cas9).
(4) After transfection, the cells were analyzed using flow cytometry to measure activity, and any cells that didn't fluoresce due to the presence of the transfection control marker were ignored.
(5) Optionally, if a library of guide RNAs is assayed at the same time, use fluorescent-assisted cell sorting (FACS) is used to sort for plasmids encoding highly active guide RNAs which are then sequenced to identify.

### Guide RNA Activity Measurement Using Plasmid Exclusion

The activity of candidate guide RNAs was measured and determined using a plasmid exclusion assay.

1) E. coli cells expressing AsCpf1 and either a guide RNA array or an empty vector were separately grown and rendered competent using standard methods.
2) Target plasmids carrying protospacers corresponding to each spacer in the array or no sequence were constructed and sequence-confirmed.
3) Target plasmids were individually transformed into the competent cells by heat shock, recovered for 2 minutes on ice and then 1 hour at 37° C. Dilutions were plated on LB agar plates containing antibiotics selecting for all three plasmids and grown for 24 hours at 37 C.
4) The number of colony-forming units for each construct was measured for each plasmid and type of competent cells. Equivalent numbers of the control plasmid lacking a protospacer indicated that both types of cells were equally competent. The ratio of colony-forming units between the two types of cells was used as a metric of Cpf1 plasmid exclusion activity.
5) Exclusion indices of >10-fold (e.g. there were consistently >10× as many colonies in the absence of the array) were recorded as active.
6) Variants that were consistently active regardless of spacer and array position were identified.
7) Optionally, a more comprehensive library-based approach can be adopted using the plasmid exclusion assay to exclude plasmids encoding an inducible toxin which kills transformed cells grown in the presence of inducer. Alternatively, variant crRNAs from the library can be paired with a spacer conferring resistance to a lytic bacteriophage, enabling active crRNAs to be isolated by exposing the bacteria to the targeted bacteriophage.

### Example 13

Studies were performed and a daisy drive system was constructed. In the study, the daisy drive system was prepared in C. elegans.

### Materials and Methods:

Daisy Drive System Preparation in C. elegans
1) A large number (over 100) of adult C. elegans were injected with all three daisy drive vectors (see FIG. 17), Pcfj601 (available directly from Addgene as Plasmid #34874) for Mos1 transposase, and pre-complexed cas9 protein from the Alt-R CRISPR-Cas9 System of IDT (Integrated DNA Technologies) to aid with integration. (See user guide on IDT website: //www.idtdna.com/pages/products/genome-editing/crispr-genome-editing/crispr-cas9-genome-editing) See Table 2 for guide RNAs used.

TABLE 2
Guide RNAs used with cas9 protein to aid with
integration of daisy drive cassettes

| Sequence | SEQ ID NO | Target gene |
|---|---|---|
| taccgtaacccgttctcgtt | 40 | CEL-cku80 |
| ttcccagaattcggcagcat | 41 | CEL-cku80 |
| ccaaagacgagaggcgtatc | 42 | CEL-fog2 |
| tggttgaccaatatcggacg | 43 | CEL-fog2 |
| atcattctctgacatgccaa | 44 | CEL-fog2 |
| tcctggcttcaagtatgtta | 45 | CEL-fog2 |

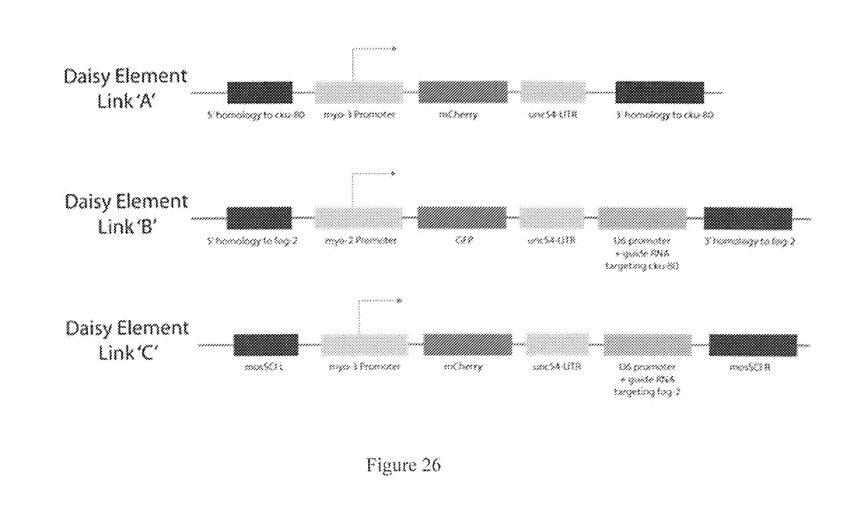

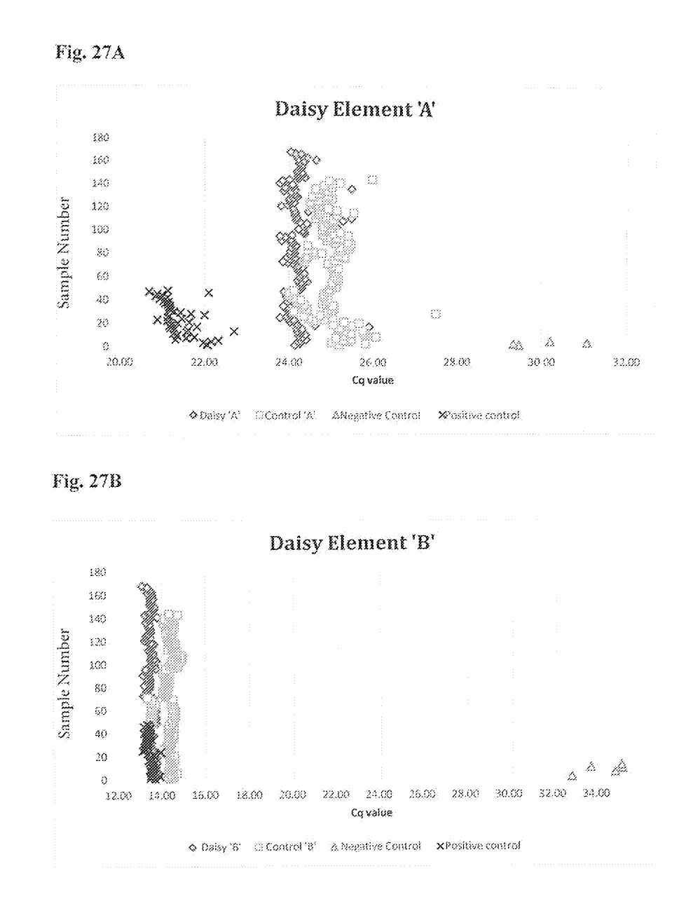

The daisy drive vectors used are shown in FIG. 26 and were as follows:

Daisy link ‘A’: which contained myo3-mCherry-unc54 UTR flanked by 500 bp of both 5′ and 3′ homology sites for Cku80.
Daisy link B: which contained Pmyo2-GFP-unc54UTR and guides targeting Cku80. It is flanked by both 5′ and 3′ homology arms to fog2.
EM-Hera: Daisy link C: which contained Prp1128+BFP+let-858 UTR+gRNA targeting fog-2. Daisy link C was the bottom link of the daisy chain and was integrated randomly into the genome via Mos1 transposase. Daisy link C was not driven by any gRNA whatsoever.
2) Worms were isolated that expressed all three colors of fluorescence (RGB) in the F1 generation.
3) Glowing F1 worms were injected a second time with vectors of the missing color to maximize probability of integration.
4) Twenty (20) RGB worms were isolated in or before the young adult stage. This step occurred before any of the 20 had laid any eggs.
5) RGB worms from step ‘3’ were divided into two groups of ten (10) worms each. One group of 10 was a control group and the worms in that group were left unchanged. The other group of 10 worms, the “Daisy” group, received injections of additional cas9 protein (10 μM) upon reaching adulthood. The injections were performed for both gonads of the worms.

It was expected that the worms in the parental generation (P0) would be heterozygous for all three gene drive elements. The injection of the cas9 protein was done to initiate “drive” activity.

6) Following step 5, the worms were left for three (3) days to lay eggs and for the F1 generation of the worms to mature.
7) The prepared C. elegans daisy drive organisms were assessed for genomic copy number and daisy drive activity. The assessment included qPCR analysis as described below.

### Assessment for Genomic Copy Number and Daisy Drive Activity

C. elegans are known to retain injected genetic material in extrachromosomal arrays for a number of generations post initial injection. Therefore simple counting of fluorescence in the F1 generation of the prepared C. elegans was not sufficient to determine drive activity. To assess drive activity in the prepared C. elegans, qPCR was used to determine the number of integrated copies of each gene. For the qPCR studies, primer pairs were designed that amplified across the junction between the inserting gene drive cassette and the existing genomic DNA. This ensured that only integrated gene drive cassettes were accounted for in the assessment. Plasmid vectors containing the target template for qPCR were diluted to an appropriate concentration of 2.42 and 4.84 zeptomoles in 1×TE buffer and used as positive controls. Negative controls were created by substituting distilled water as amplification template.

The appropriate concentration of DNA used for positive controls are matched to the theoretical concentration of single worm DNA extractions according to the following reasoning. An adult C. elegans contains 959 (diploid) somatic cells and 1000-2000 germ cells (haploid). On this basis, it was assumed that a homozygote worm contained 2918 copies of the gene drive cassette and that a heterozygote worm contained 1459 copies. This translates to 4.84 and 2.42 zeptomoles respectively. 168 worms were picked from each of the F1 generation progeny from the two experiments, the ‘daisy’ and ‘control’ groups, described above. Each worm was suspended individually in 10 uL of lysis buffer following the Williams et al. protocol (Williams B D, et. al., (1992) Genetics July; 131(3):609-24, and //github.com/mfitzp/theolb/blob/master/molecular-biology/c-elegans-single-worm-per.rst). The worms were then flash-frozen in an ethanol/dry ice slurry. The frozen worms were heated to 65° C. and then heated to 95° C. The resulting prepared single worm DNA extract was stored at 4° C. until used the qPCR procedure.

qPCR Procedure

qPCR is performed on an bio-Rad qPCR cfx384 instrument with the intensity threshold for Cq set at 0.2. The same program is used for all qPCR experiments: 95° C. for 3 minutes followed by 40 cycles of (95° C. for 10 seconds and 55° C. for 1 minute). qPCRs are performed using the KAPA Sybr Fast qPCR kit following manufacturer's instructions. For the 384-well plate format we used, each qPCR reaction was made up of 5 μL of Kapa Master Mix, 0.2 μL of each 10 μM primer, 2.6 μL of distilled water, and 2 μL of genomic DNA extract from each of the single worms. For positive and negative controls, the worm genomic DNA extract was replaced with either plasmid vectors diluted to the appropriate concentration or water as described below.

For positive controls, 2.42 and 4.84 zeptomoles of positive control vectors were added to row ‘A’ of a 384 well plate to represent homo and heterozygosity. The remaining wells were filled with single worm DNA extract from the F1 generation. Thus, each 96-well plate represented a random sampling of progeny from two individual parents. It was expected that a ˜1 cycle difference in time to 0.2 fluorescence intensity on qPCR between heterozygotes and homozygotes of the daisy drive cassettes. It was expected that most, if not all of the fluorescing worms of the “control” group to be heterozygotes. It was expected that most, if not all, of the fluorescing worms of the “daisy” group to be homozygotes.

The Daisy Element ‘A’, or the ultimate link of the prepared daisy chain, was expected to exhibit the behavior described above only if the daisy drive system was working as designed.

TABLE 3
Mean ‘Cq’ values of the data groups.

| Sample ID | Mean Cq | StdDev | n |
|---|---|---|---|
| Daisy ‘A’ | 24.20525 | 0.300189 | 168 |
| Control ‘A’ | 24.966 | 0.456865 | 130 |
| Positive control ‘A’ | 21.40508 | 0.428471 | 48 |
| Daisy ‘B’ | 13.50748 | 0.145702 | 168 |
| Control ‘B’ | 14.32553 | 0.288308 | 144 |
| Positive control ‘B’ | 13.4535 | 0.16743 | 48 |
| Daisy ‘C’ | 24.43035 | 0.463744 | 136 |
| Control ‘C’ | 24.40731 | 0.484118 | 156 |
| Positive control ‘C’ | 23.81716 | 4.157946 | 48 |

Note:
‘n’ value for Control ‘A’ is lower than 168 because a number of samples failed to run and were excluded from the mean calculation. Data points from both “daisy” and “control” groups that overlap with negative controls were removed from data table.

### Results

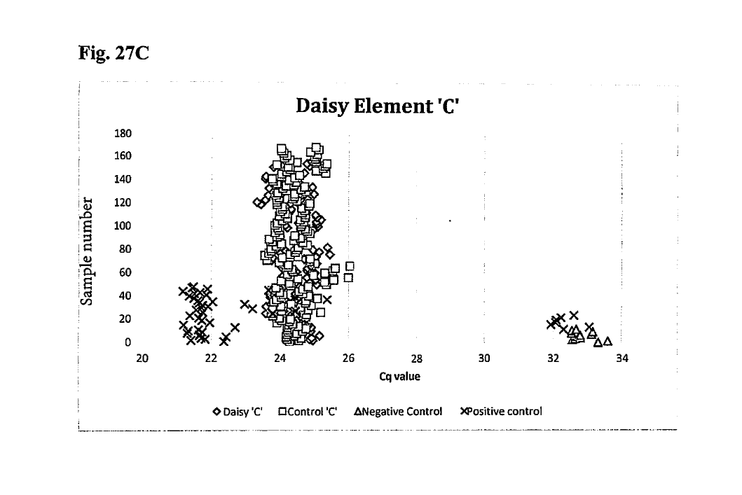

Results of the qPCR are shown in FIGS. 27a-C and in Table 3, and demonstrated that the daisy drive system as described was working as designed. The data showed that almost all of the fluorescing “control” group worms were heterozygotes and almost all of the fluorescing worms of the “daisy” group were homozygotes.

### Example 14

### Methods for Enhanced Daisy Drive Precision

In an ideal world, it would be possible to constrain the effects of a gene drive system to the legal side of a political area or boundary inside of which it is desired to contain the activity of the drive system. In the real world, daisy drives may keep alterations local, but they may not be as precise as is desirable in certain circumstances due to mixed populations of wild-types and heterozygotes at the boundaries of the desired area, some organisms with genetic changes will inevitably end up on the other side unless there is a very large buffer zone within the consenting community, which in turn will not enjoy the desired benefits of the gene drive system.

It has now been determined that enhanced precision daisy drive systems can be prepared in a manner that includes underdominance activity in order to keep population-genetic boundaries clear and distinct, enabling the daisy drive system activity to closely conform to community-determined regions, areas, and boundaries. Such methods of regional constraint ensures that hybridization between wild-type and engineered organisms results in fewer progeny and will select against whichever version is currently less common in the population, thereby keeping the engineered and wild-type populations pure. By reducing the fitness of altered individuals within wild-type populations and wild-type individuals within altered populations, the boundary between these populations becomes sharper. This increased sharpness permits the boundary to be adjusted to closely conform to political areas and boundaries by targeted releases of appropriate wild-type or daisy drive organisms.

Studies show that an important aspect to combining daisy drive with underdominance is to ensure that the underdominance effect is triggered when the daisy drive activity ceases, and not before. This is important because daisy drive organisms are always rare relative to wild-type when released; therefore, if underdominance took effect immediately, the daisy drive organisms would be strongly selected against.

Experiments and tests are performed that comprise designing, constructing, and using enhanced precision gene drive systems that result in increased specificity of a daisy chain gene drive system of the invention respect to geographic areas, regions, and boundaries—as compared to a gene drive system that lacks the enhanced precision elements. Tests are performed to assess the efficacy of such methods to constrain the effects of a gene drive system within a region and/or boundary. Enhanced daisy chain gene drives of the invention are prepared and are used to produce regionally localized changes in organisms and populations with regional precision of the released daisy chain gene drive in a community or other political region.

In one example, it is undesirable to have a released daisy chain gene drive present or active in an area other than the area for which the release is intended. An area determined or selected to not include the released daisy chain gene drive or its direct effects may be adjacent to, or in close physical proximity to an area for which a release of the daisy chain gene drive is intended. In one such situation, a buffer zone is used to reduce and/or prevent the presence of the released daisy chain gene drive in the unintended region or area. Such buffer areas are included within the area in which release is intended, but the daisy chain gene drive system is not released in the buffer zone. A community desires to utilize release of a daisy chain gene drive system in a first area, but limits entry of the system into a second area, for example in an adjacent community that does not consent to the presence of the daisy chain gene drive system. A buffer region is determined in the first area and the daisy chain gene drive system released into the first region, except in the buffer region portion of the first region.

In a second study, a strategy to increase precision of localization of a daisy chain gene drive system is constructed and tested. A precision containment daisy chain gene drive system is constructed and tested in which the daisy drive system includes underdominance components. The precision daisy chain gene drive system keeps population-genetic boundaries clear and distinct, enabling them to closely conform to regional and area boundaries. Precision containment methods of the invention that ensure that hybridization between wild-type and engineered organisms results in fewer progeny—select against whichever version of organism is currently less common in the population, thereby keeping the engineered and wild-type populations pure. Methods of the invention are used to reduce the fitness of altered individuals within wild-type populations and wild-type individuals within altered populations, resulting in the boundary between these populations being sharper and more distinct than in the absence of the precision aspects of the daisy chain gene drive system, permitting the boundary to be adjusted to closely conform to one or more geographic, community, and desirable areas and boundaries by targeted releases of wild-type or daisy drive organisms.

Studies are performed to assess accomplishing underdominance, and precision gene drives, by creating a chromosomal rearrangement that swaps the positions of two or more essential genes. Normal chromosomal segregation during meiosis therefore assures that matings between heterozygotes and wild-types result in only 50% progeny survival (FIG. 17A). The same effect occurs when heterozygotes mate with homozygous altered organisms or even with other heterozygotes. Experiments in which the locations of essential genes are swapped thereby resulting in an underdominance effect in a daisy drive system.

Studies are also performed to assess an underdominance effect in a daisy drive system of the invention, without directly replacing one gene with another; by inserting guide RNAs that eliminate the other gene and a re-coded copy that will rescue individuals that inherit it. Such methods of the invention result in precision effects and can comprise including the inserted cassettes in different locations in the genome.

CRISPR-based underdominance daisy drive methods of the invention are tested. These methods utilize the fact that a daisy drive payload element normally targets and recodes a gene important for fitness anyway, for example, though not intended to be limiting, a haploinsufficient gene. A diagram of a precision daisy drive method is shown in FIG. 17A-B, which illustrates a situation in which at least two such payload elements are created (for example: A and U in FIG. 17B). Genetic locus A normally has haploinsufficient gene hA; while genetic locus U normally has haploinsufficient gene hU. In the daisy drive version, element A has guide RNAs targeting hU as well as a recoded copy, hU′, in place of the hA. Similarly, element U has guide RNAs targeting hA as well as a recoded copy, hA′, in place of hU. In other words, these elements catalyze the replacement of the wild-type gene at their own locus with a recoded version of the other locus' gene. The genes swap positions. When the drive nuclease is present (element B), drive occurs in both places, thereby replacing hA with hU′ and hU with hA′. All offspring inherit one of each and consequently are guaranteed to be fine. But when there is no drive nuclease, i.e. the daisy drive has run out of genetic fuel (elements), offspring inherit either hA or hU′ and either hU or hA′, meaning half of them lack a working copy of a haploinsufficient gene and consequently are very unfit. In other words, underdominance occurs only when the daisy drive runs out of elements and stops.

Each of the above-described system of the invention, certain embodiments of which are illustrated in FIG. 17A-B, are prepared, introduced into cells and organisms, and are utilized in methods of the invention. Means for designing constructing, integrating, and implementing such systems of the invention as well as preparing organism strains and releasing organisms of such strains, etc. that include such systems of the invention is carried out using the teaching presented herein, and in certain instances in conjunction with methods, components, and/or elements known in the art.

### Example 15

Underdominance is also accomplished using methods of the invention referred to herein interchangeably as: “toxin-antitoxin” and “killer-rescue” systems. FIG. 17C-J provides illustrations of various embodiments of toxin-antitoxin and killer-rescue systems of the invention.

Another assessment of a precision, underdominance daisy drive system and method of the invention is performed with an RNAi-based toxin-antitoxin underdominance daisy drive system. The system is prepared using components described in Akbari et al 2013 Current Biology Volume 23, Issue 8, p 671-677, the content of which is incorporated herein by reference in its entirety. Components, sequences, and methods disclosed by Akbari et al., including but not limited to the uDmel locus, are used in a precision daisy drive underdominance systems and the system tested. In one study, one UDmel locus is incorporated into element A of a daisy drive, and the other locus into element U. In the active daisy drive, all offspring inherit the re-coded copy and are fine; e.g. underdominance does not take place. When the tested daisy drive runs out of elements, Mendelian segregation occurs, meaning not all offspring inherit the protective copy. Males transmit both copies as normal.

Another assessment of a precision underdominance daisy drive system and method of the invention is carried out using an RNAi-based toxin-antitoxin underdominance daisy drive system. The system is prepared such that it includes RNAi-based toxin-antitoxin underdominance without a maternal effect. Systems tested include in a daisy drive system of the invention, a copy of an underdominance cassette that knocks down a haploinsufficient gene via RNAi and provides a recoded copy, in payload element A, and another in payload element U. Assessments are performed using a system prepared using components described in Reeves et al., 2014 PLoS, http://dx.doi.org/10.1371/journal.pone.0097557, the content of which is incorporated herein by reference in its entirety. Components, sequences, and methods disclosed Reeves et al., (for example in FIG. 1, page 1-2, etc.) are used to construct a precision daisy drive underdominance system and the system is tested.

A precision RNAi-based toxin-antitoxin underdominance daisy drive system is prepared that includes at least one copy of a cassette such as that disclosed in Reeves, which knocks down a haploinsufficient gene via RNAi and provides a recoded copy in payload element A, and another in payload element U. As long as all offspring inherit a recoded copy of A and U because the drive is active, the offspring are viable. When the prepared precision underdominance daisy drive is no longer active, any offspring with wild-type that do not inherit a copy of both the A and U elements are not viable. This is consequently more effective than certain other methods, because only ¼ of the offspring will survive.

Another precision toxin-antitoxin underdominance daisy drive system is prepared and tested in the zygote of an organism. The system includes a zygotically active form of CRISPR (e.g. not using the germline-active form employed in the daisy drive). In this system, instead of relying on RNAi to suppress expression of essential or haploinsufficient genes, CRISPR is used as a toxin and much more reliably disrupts the essential or haploinsufficient genes. In some tests, the antitoxin is a re-coded version of the targeted gene that is not disrupted by the CRISPR system.

Another non-limiting example of an underdominance daisy drive method of the invention is RNAi-based toxin-antitoxin underdominance daisy drive methods. For example, Akbari et al 2013 Current Biology Volume 23, Issue 8, p 671-677, the content of which is incorporated herein by reference in its entirety, describes a two-locus UDmel method in which maternal deposition of inhibitory RNAi molecules targeting an essential gene renders progeny nonviable unless they inherit a recoded copy of that gene that is not inhibited. Components, sequences, and methods disclosed by Akbari et al., including but not limited to the uDmel locus, can be used in certain embodiments of daisy drive underdominance systems and methods of the invention. For example, one UDmel locus can be incorporated into element A of a daisy drive, and the other locus into element U. As long as the daisy drive is active, all offspring will inherit the recoded copy and be fine; e.g. underdominance will not take place. Once the daisy drive runs out of elements, Mendelian segregation will occur, meaning not all offspring will inherit the protective copy. Males will transmit both copies as normal.

Another non-limiting example of an RNAi-based toxin-antitoxin underdominance daisy drive method of the invention includes RNAi-based toxin-antitoxin underdominance without a maternal effect. An embodiment of such a method of the invention may include in a daisy drive system of the invention, a copy of an underdominance cassette that knocks down a haploinsufficient gene via RNAi and provides a recoded copy, in payload element A, and another in payload element U. An example of an underdominance cassette that may be used in an RNAi-based toxin-antitoxin underdominance daisy drive method of the invention is set forth in Reeves et al., 2014 PLoS, http://dx.doi.org/10.1371/journal.pone.0097557, the content of which is incorporated herein by reference in its entirety. Components, sequences, and methods disclosed Reeves et al., (for example in FIG. 1, page 1-2, etc.) can be used in certain embodiments of daisy drive underdominance systems and methods of the invention. For example, some embodiments of RNAi-based toxin-antitoxin underdominance daisy drive systems of the invention include at least one copy of a cassette such as that disclosed in Reeves, which will knock down a haploinsufficient gene via RNAi and will provide a recoded copy in payload element A, and another in payload element U. As long as all offspring inherit a recoded copy of A and U because the drive is active, the offspring are viable. When the embodiment of the underdominance daisy drive of the invention is no longer active, any offspring with wild-type that do not inherit a copy of both the A and U elements will not be viable. This is consequently more effective as only ¼ of the offspring will survive.

Another non-limiting example of a toxin-antitoxin underdominance daisy drive method of the invention in the zygote of an organism comprises using a zygotically active form of CRISPR (e.g. not using the germline-active form employed in the daisy drive). In certain embodiments of enhanced precision daisy chain systems and methods of the invention, instead of relying on RNAi to suppress expression of essential or haploinsufficient genes, CRISPR is used as a toxin to much more reliably disrupt the essential or haploinsufficient genes. In some embodiments of such systems and methods, the antitoxin is a recoded version of the targeted gene that is not disrupted by the CRISPR system.

FIG. 17C-J illustrates certain embodiments of the above-described toxin-antitoxin systems. FIG. 17C illustrates a CRISPR-based killer-rescue system, also referred to as: a toxin-antitoxin system, generated by inserting a copy of a haploinsufficient gene next to the payload and disrupting the wild-type copy elsewhere in the genome. Offspring that inherit a disrupted version without the new copy perish. Offspring that inherit more than the normal two copies may or may not be highly unfit due to the extra expression; if they are reasonably fit then the payload will spread to a limited extent. The net effect is a form of underdominance. FIG. 17D illustrates a killer-rescue system generated by a daisy drive system, which encodes the germline-expressed nuclease in the B element, a recoded copy of the haploinsufficient gene along with the payload in the A element, and guide RNAs that disrupt the wild-type copy in the U locus. Daisy drive propagation occurs as normal because all offspring inherit a recoded copy and a broken copy until the nuclease is no longer present. At this point the killer-rescue/toxin-antitoxin system becomes active and selects for homozygosity at A and U. FIG. 17E illustrates a more powerful killer-rescue system for which heterozygotes produce fewer progeny that is generated by encoding two different copies of a haploinsufficient gene next to the payload and disrupting the wild-type copy. Offspring that inherit either disrupted version without the payload perish. Offspring that inherit more than the normal two copies may or may not be highly unfit due to the extra expression; this may cause the payload to spread if they are reasonably fit. The net effect is a stronger form of underdominance. FIG. 17F illustrates that a stronger killer-rescue system can also be generated by a daisy drive system so that it manifests after the drive halts. The stronger underdominance is more effective at selecting for homozygosity at A, U, and V (the locus encoding the second haploinsufficient gene). FIG. 17G-I provides diagrams of family trees demonstrating the underdominance effect and possible limited spread caused by the killer-rescue/toxin-antitoxin system. FIG. 17J illustrates a CRISPR-based toxin-antitoxin system that generates a Medea effect: any offspring that do not inherit the Medea element perish due to lack of a haploinsufficient gene. Because it is expected that Medea elements will be self-sustaining in the event of density-dependent selection, in some embodiments of the invention, they are generated without adding a daisy drive. In certain circumstances, a non-limiting example of which is when a goal is to release very few organisms in order to exceed the threshold level for continued spread, a daisy drive system can be added. Adding a daisy drive system can be done by including another element (B) that encodes guide RNAs that drive the Medea element (not shown).

Each of the above-described system of the invention, certain embodiments of which are illustrated in FIG. 17C-J, are prepared, introduced into cells and organisms, and are utilized in methods of the invention. Means for designing constructing, integrating, and implementing such systems of the invention as well as preparing organism strains and releasing organisms of such strains, etc. that include such systems of the invention is carried out using the teaching presented herein, and in certain instances in conjunction with methods, components, and/or elements known in the art.

### Example 16

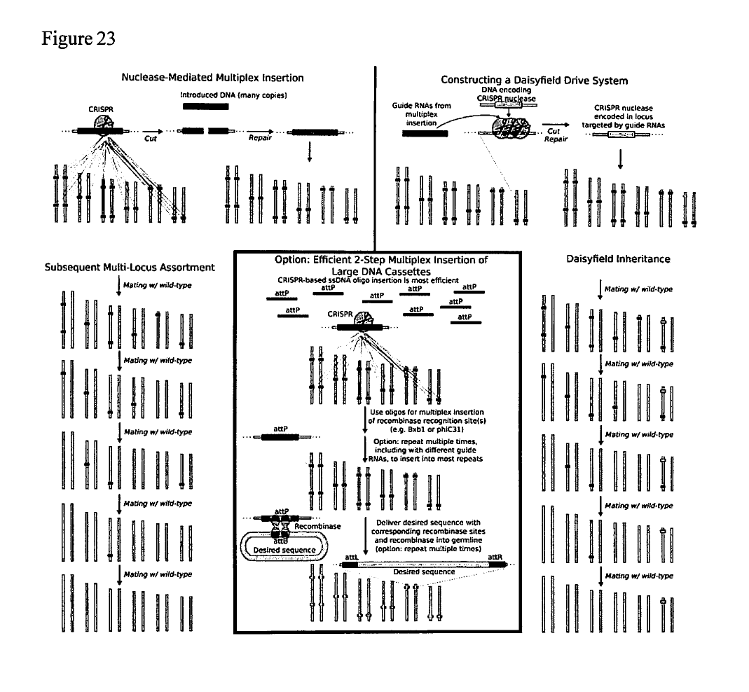

Organisms and strains of organism are prepared with components as illustrated in FIG. 23 using methods described elsewhere herein and standard art-known procedures.

In one study, nuclease-mediated multiplex insertion is performed. An embodiment of a method for this study is shown in FIG. 23, left hand side. A Daisyfield Drive system is constructed using steps illustrated in FIG. 23, right hand side.

An additional procedure is carried out that includes an efficient 2-step multiplex insertion of large DNA cassettes. An example of the process is illustrated in the center of FIG. 23. Organisms prepared using the methods in this example are released into a wild and their effectiveness is tested including survival, reproduction, and impact on numbers of wild type organism of the same species that are in the wild environment.

### Example 17

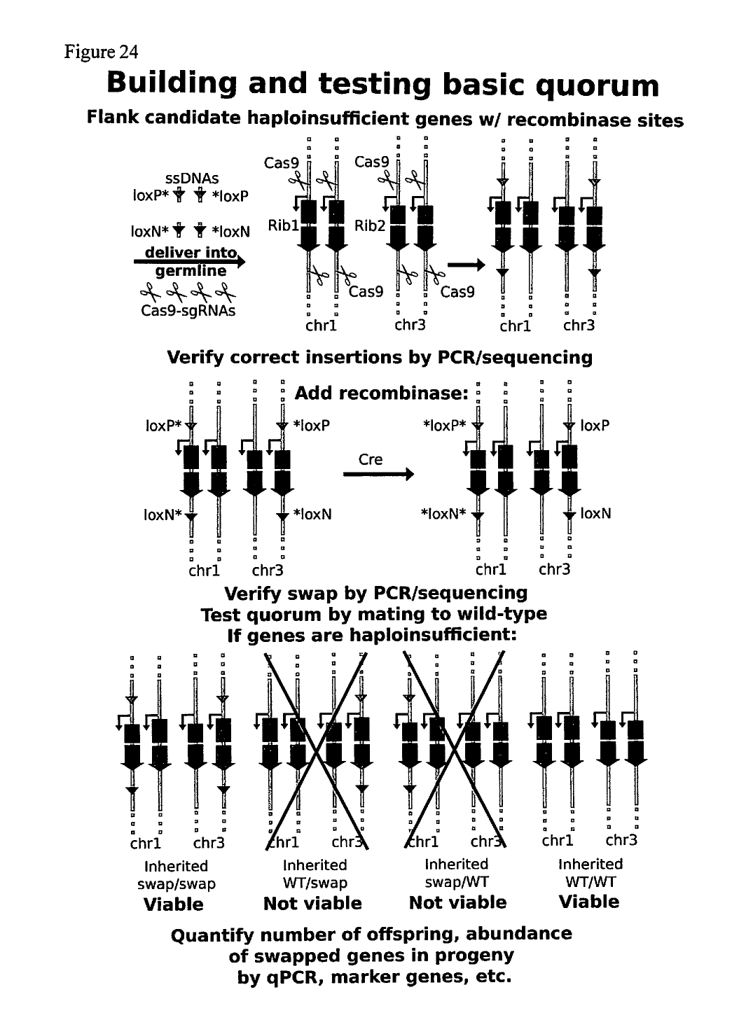

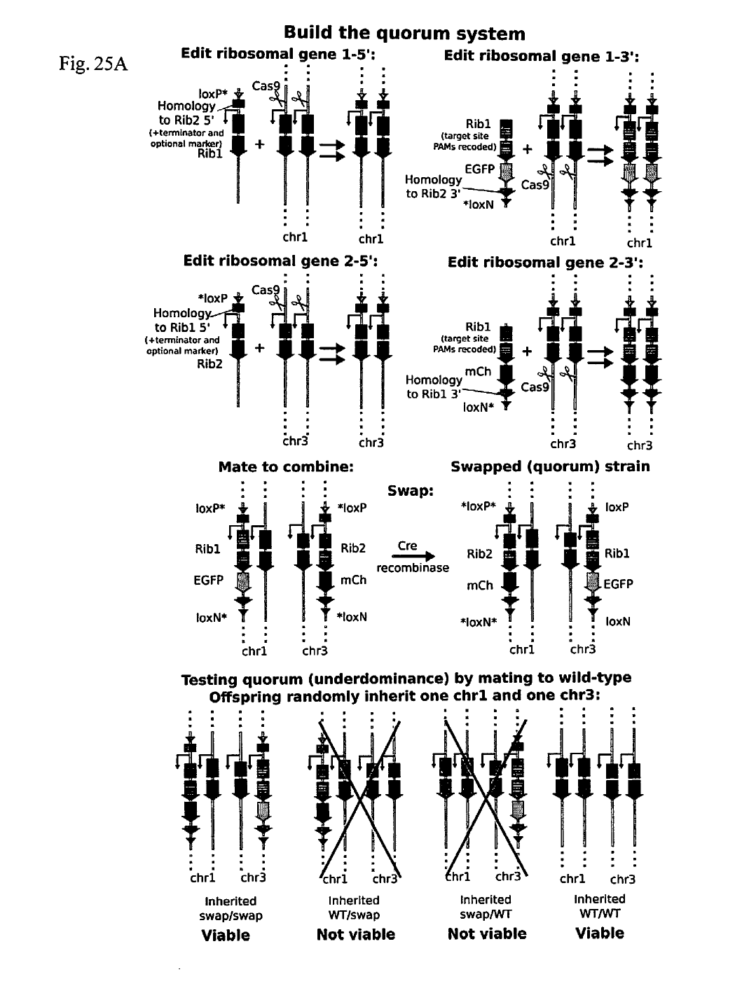

Organisms and strains of organism are prepared with components as illustrated in FIG. 24 using methods described elsewhere herein and standard art-known procedures. An embodiment of a procedure is shown in FIG. 24, which provides a schematic diagram of steps used to build and test this basic quorum. The diagram shows how selected candidate haploinsufficient genes are flanked with recombinase sites. FIG. 24 indicates that the location and presence of correct insertions can be assessed and verified using standard methods such as amplification methods (for example, PCR) and sequencing). FIG. 24 also illustrates the effect of adding a recombinase, which results in swapping of the genes. The completion of the expected swap can be verified using standard methods such s amplification and sequencing methods. FIG. 24 illustrates crossing of a prepared engineered organism with a wild-type version of the organism and the expected results from such a cross. FIG. 24 indicates various types of assay methods that can be performed to determine the efficacy of the basic quorum. Organisms are prepared using the methods in this example and are released into the wild. Their impact on one or more wild populations is with assessment of factors including, but not limited to: survival, reproduction, and impact on numbers of wild type organism of the same species that are in the wild environment.

### Example 18

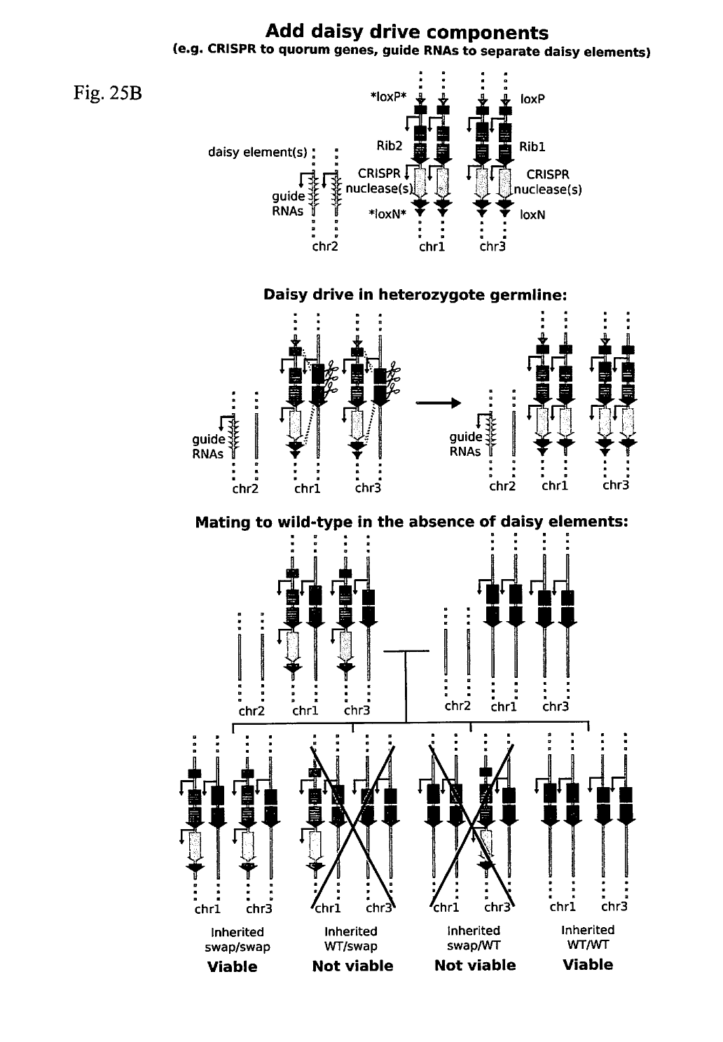

Organisms and strains of organism are prepared with components as illustrated in FIGS. 25a-B using methods described elsewhere herein and standard art-known procedures. FIG. 25A-B provides a schematic diagram of methods of building an embodiment of a quorum system of the invention and also including daisy drive components in the quorum genes. FIG. 25A illustrates studies that include editing ribosomal genes, mating the organisms that include the edited genes, swapping (exchanging) the introduced DNA and testing quorum underdominance by mating the engineered organism to wildtype and assessing viability of their offspring. FIG. 25B illustrates procedures in which daisy drive components are added into the system, for example, CRISPR is added to quorum genes along with guide RNAs to separate daisy elements. FIG. 25B shows results of inclusion of the daisy drive in heterozygote germline, and results of mating in the absence of daisy elements. Organisms are prepared using the methods in this example and are released into the wild. Their impact on one or more wild populations is with assessment of factors including, but not limited to: survival, reproduction, and impact on numbers of wild type organism of the same species that are in the wild environment.

### Statement for all Examples

Means for designing constructing, integrating, and implementing such systems of the invention as well as preparing organism strains and releasing organisms of such strains, etc. that include such systems of the invention is carried out using the teaching presented herein, and in certain instances in conjunction with methods, components, and/or elements known in the art.

### EQUIVALENTS

Although several embodiments of the present invention have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present invention. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present invention is/are used. Those skilled in the art will recognize, or be able to ascertain using no more than routine experimentation, many equivalents to the specific embodiments of the invention described herein. It is, therefore, to be understood that the foregoing embodiments are presented by way of example only and that, within the scope of the appended claims and equivalents thereto; the invention may be practiced otherwise than as specifically described and claimed. The present invention is directed to each individual feature, system, article, material, and/or method described herein. In addition, any combination of two or more such features, systems, articles, materials, and/or methods, if such features, systems, articles, materials, and/or methods are not mutually inconsistent, is included within the scope of the present invention.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.” The phrase “and/or,” as used herein in the specification and in the claims, should be understood to mean “either or both” of the elements so conjoined, i.e., elements that are conjunctively present in some cases and disjunctively present in other cases. Other elements may optionally be present other than the elements specifically identified by the “and/or” clause, whether related or unrelated to those elements specifically identified, unless clearly indicated to the contrary.

All references, patents and patent applications and publications that are cited or referred to in this application are incorporated herein in their entirety herein by reference.

What is claimed:

1. A method of preparing an engineered organism, the method comprising: inserting one or more DNA cassettes each comprising an independently preselected DNA sequence into a plurality of repeated regions in the genome of an organism of a strain to prepare a first engineered organism, wherein a means of inserting the preselected DNA sequence comprises at least one of:
a) using transposons to pseudo-randomly incorporate a plurality of copies of the preselected DNA sequence into the genome of the organism; and
b) using a nuclease-class enzyme to cut one or more strands of a predetermined natural sequence repeat in the genome of the organism and inducing homologous recombination of the preselected DNA sequence with the predetermined natural sequence.

2. The method of claim 1, wherein the DNA cassette is a site DNA cassette comprising DNA of one or more small recombinase sites.

3. The method of claim 2, wherein the DNA cassette is an insertion DNA cassette, and the method further comprises inserting one or more of the insertion DNA cassettes into the site DNA cassettes harboring the one or more small recombinase sites using one or more of an appropriate recombinase enzyme.

4. The method of claim 1, wherein the DNA cassette is an insertion DNA cassette and comprises a DNA sequence encoding a desired organism trait, and the method further comprises:
c) preparing a plurality of the engineered organism comprising a plurality of one or more inserted DNA sequences conferring the desired organism trait;
d) releasing the plurality of the prepared engineered organisms, wherein the release introduces the desired trait into a local population of the organism of the strain.

5. The method of claim 1, wherein, the DNA cassette is an insertion DNA cassette, and two or more of the insertion DNA cassettes are inserted, wherein: (i) a first insertion DNA cassette comprises one or more CRISPR components and a plurality of the first insertion DNA cassettes is inserted into the plurality of repeated regions in the genome of the organism; (ii) a second insertion DNA cassette is inserted into a single site in the genome of the organism; wherein the second insertion DNA cassette comprises DNA encoding: (a) one or more cargo genes, and optionally encoding (b) an independently selected CRISPR component that differs from that in the first insertion DNA cassette.

6. The method of claim 5, wherein the CRISPR components comprise a nuclease and the method further comprises the nuclease inducing conversion of one or more of a germline cell of the organism that are heterozygous for the second insertion DNA cassette into homozygotes by nuclease-mediated cutting and repair by homologous recombination, thereby copying the second insertion DNA cassette.

7. The method of claim 6, wherein the first insertion cassette comprises a DNA encoding one or more guide RNAs and a plurality of the first insertion cassette is inserted throughout the genome of the organism, and the second insertion cassette comprises a DNA encoding the nuclease gene(s) and one or more cargo genes, and the second insertion cassette is copied in the presence of at least one guide RNA cassette.

8. The method of claim 6, wherein the first insertion cassette comprises a DNA encoding the nuclease gene and a plurality of the first insertion cassette is inserted throughout the genome of the organism, and the second insertion cassette comprises one or more guide RNAs and one or more cargo genes, and the second insertion cassette is copied in the presence of at least one copy of the nuclease gene.

9. The method of claim 6, wherein the first insertion cassette comprises a DNA encoding one or more guide RNAs and one or more corresponding nuclease enzymes and a plurality of the first insertion cassette is inserted throughout the genome of the organism, and the second insertion cassette comprises a DNA encoding one or more cargo genes, and the second insertion cassette is copied in the presence of at least one copy of the first insertion cassette.

10. The method of claim 5, further comprising generating a transgenic strain of the engineered organism wherein the genome of the organism comprises a plurality of copies of first insertion cassette comprising the CRISPR components and one copy of the second insertion DNA cassette comprising one or more cargo genes.

11. The method of claim 10, further comprising, releasing a plurality of organisms of the transgenic strain, wherein the release efficiently introduces copies of the second insertion DNA cassette into a local population of organisms of the strain.

12. The method of claim 1, wherein the nuclease-class enzyme is a nickase or a nuclease.

13. The method of claim 1, wherein a plurality is: 2 or more, 3 or more, 4 or more, 5 or more, or 6 or more.

14. A method of generating a threshold-dependent gene drive system by engineered underdominance in a population of organisms, comprising exchanging in one or more organisms in the population, positions of a first haploinsufficient gene on a first chromosome in a cell of the organism with a second haploinsufficient gene in an unlinked locus, such as on a second chromosome in the cell of the organism.

15. The method of claim 14, wherein the first and second haploinsufficient genes are ribosomal genes, neither the first nor the second haploinsufficient genes are ribosomal genes, or only one of the first and the second haploinsufficient genes is a ribosomal gene.

16-29. (canceled)

30. A method of generating a toxin-antitoxin gene drive system, comprising
(a) inserting into a genome of an organism one or more DNA cassettes encoding a toxin in the form of one or more preselected CRISPR nuclease genes, one or more corresponding guide RNAs, and appropriate expression signals, wherein when expressed, the preselected CRISPR genes cut and disrupt a target gene required for viability or fertility of the organism, and
(b) inserting into the genome of the organism one or more DNA cassettes encoding one or more cargo genes and an antitoxin comprising at least one copy of one or more recoded versions of the target gene, wherein the recoded versions of the target gene comprise one or more sequence modifications in the nucleic acid sequence of the target gene wherein the one or more modifications prevent cutting of the recoded gene by the nuclease and do not alter the amino acid sequence of the expressed recoded target gene from that of the expressed target gene, and wherein expressing the one or more recoded target genes is sufficient to rescue viability or fertility.

31-41. (canceled)

42. A method of constructing a gene drive system that combines nuclease-induced copying with threshold-dependence, the method comprising:
(a) inserting into a genome one or more first DNA cassettes, wherein the first DNA cassettes comprises sequences encoding one or more components of a threshold-dependent gene drive system, and
(b) inserting into the genome one or more second DNA cassettes, wherein the second DNA cassettes comprises sequences encoding one or more components of a nuclease-based gene drive system, wherein the nuclease-based drive system is designed to cut one or more target DNA sequences in at least one germline cell of a heterozygote organism resulting in copying of the one or more first DNA cassettes, and wherein the first DNA cassettes optionally further comprise sequences encoding one or more cargo genes.

43-94. (canceled)

95. The method of claim 1, wherein the organism is a vertebrate, optionally is a mammal, and optionally is a rodent.

96. The method of claim 1, wherein the organism is an invertebrate.

97. The method of claim 1, wherein the organism is of a strain of: Rattus rattus, Aedes aegypti, Culex quinquefasciatus, or Anopheles gambiae.


SEQUENCE LISTING (46 sequences)

SEQ ID NO: 1 - protein, 1368 aa, S. pyogenes [sequence omitted]

SEQ ID NO: 2 - DNA, 76 bp, artificial sequence
  synthetic oligonucleotide
gttttagagctagaaatagcaagttaaaataaggctagtccgttatcaacttgaaaaagtggcaccgagtcggtgc

SEQ ID NO: 3 - DNA, 76 bp, artificial sequence
  synthetic oligonucleotide
gttctagagagcgggagctcaagttagaataaggctagtccgtattcagtgcgggagcacggcaccgattcggtgc

SEQ ID NO: 4 - DNA, 90 bp, artificial sequence
  synthetic oligonucleotide
gttcgagaggacgagagtccaagttcgaataaggcagtgatttttaatccagtccgttatcacagggagacctgggcaccgagtcggtgc

SEQ ID NO: 5 - DNA, 90 bp, artificial sequence
  synthetic oligonucleotide
gttccagagtcgggaacgacaagttggaataaggcagtgtattatataccagtccgttatcatgccggaaggcaggcaccgattcggtgc

SEQ ID NO: 6 - DNA, 92 bp, artificial sequence
  synthetic oligonucleotide
gttgtagagcgtagaaatacgcaagttacaataaggcagtgaattaaattccagtccgttatcaagcggaaacgctggcaccgagtcggtgc

SEQ ID NO: 7 - DNA, 94 bp, artificial sequence
  synthetic oligonucleotide
gttgcagagacacgggagtgtcaagttgcaataaggcagtgtttattaaaccagtccgttatcagacgtgggaacgtcggcaccgattcggtgc

SEQ ID NO: 8 - DNA, 80 bp, artificial sequence
  synthetic oligonucleotide
gttggagaggcatgagaatgccaagttccaataaggctagtccgtacacacctaggagactaggggcaccgagtcggtgc

SEQ ID NO: 9 - DNA, 80 bp, artificial sequence
  synthetic oligonucleotide
gtcttagagtgtgggaacacacaagttaagataaggctagtccgttatcatcaggggaacctgaggcaccgattcggtgc

SEQ ID NO: 10 - DNA, 80 bp, artificial sequence
  synthetic oligonucleotide
gtcctagagccatgaaaatggcaagttaggataaggctagtccgtattcaacgctgaaaagcgtggcaccgagtcggtgc

SEQ ID NO: 11 - DNA, 94 bp, artificial sequence
  synthetic oligonucleotide
gtccgagagatccgggaggatcaagttcggataaggcagtcatttttaatgcagtccgttatcagctcagggatgagcggcaccgattcggtgc

SEQ ID NO: 12 - DNA, 94 bp, artificial sequence
  synthetic oligonucleotide
gtcccagaggttcgagagaaccaagttgggataaggcagtctattatatagcagtccgttatcacgaacgagagttcgggcaccgagtcggtgc

SEQ ID NO: 13 - DNA, 94 bp, artificial sequence
  synthetic oligonucleotide
gtcgtagagttggggaaccaacaagttacgataaggcagtcaattaaattgcagtccgttatcatctcgggaacgagaggcaccgattcggtgc

SEQ ID NO: 14 - DNA, 96 bp, artificial sequence
  synthetic oligonucleotide
gtcgcagagcatctgaaaagatgcaagttgcgataaggcagtctttattaaagcagtccgttatcaagctcgggagagctggcaccgagtcggtgc

SEQ ID NO: 15 - DNA, 82 bp, artificial sequence
  synthetic oligonucleotide
gtcggagagaacaggggactgttcaagttccgataaggctagtccgtacacagtcgtgagaacgacggcaccgattcggtgc

SEQ ID NO: 16 - DNA, 82 bp, artificial sequence
  synthetic oligonucleotide
gtgttagagggatagagatatcccaagttaacataaggctagtccgttatcactgcaggaatgcagggcaccgagtcggtgc

SEQ ID NO: 17 - DNA, 82 bp, artificial sequence
  synthetic oligonucleotide
gtgctagagtacgtggaaacgtacaagttagcataaggctagtccgtattcatgtcggaaacgacaggcaccgattcggtgc

SEQ ID NO: 18 - DNA, 96 bp, artificial sequence
  synthetic oligonucleotide
gtgcgagagcttacgaaagtaagcaagttcgcataaggcagtgatattttatccagtccgttatcaacagcgggagctgtggcaccgagtcggtgc

SEQ ID NO: 19 - DNA, 96 bp, artificial sequence
  synthetic oligonucleotide
gtgccagagagtaggggactactcaagttggcataaggcagtgtaatatttaccagtccgttatcaggtcagagatgaccggcaccgattcggtgc

SEQ ID NO: 20 - DNA, 96 bp, artificial sequence
  synthetic oligonucleotide
gtggtagaggacttgagaaagtccaagttaccataaggcagtgaaattatttccagtccgttatcacacagggaactgtgggcaccgagtcggtgc

SEQ ID NO: 21 - DNA, 96 bp, artificial sequence
  synthetic oligonucleotide
gtggcagagtcatcggaagatgacaagttcccataaggcagtgttaatttaaccagtccgttatcatccgagaaatcggaggcaccgattcggtgc

SEQ ID NO: 22 - DNA, 82 bp, artificial sequence
  synthetic oligonucleotide
gtgggagagccaaagaaatttggcaagttcccataaggctagtccgtacacaaggtcgggagacctggcaccgagtcggtgc

SEQ ID NO: 23 - DNA, 76 bp, artificial sequence
  synthetic oligonucleotide
gttcgagaggacgagagtccaagttcgaataaggccagtccgttatcacagggagacctgggcaccgagtcggtgc

SEQ ID NO: 24 - DNA, 76 bp, artificial sequence
  synthetic oligonucleotide
gttccagagtcgggaacgacaagttggaataaggcaagtccgttatcatgccggaaggcaggcaccgattcggtgc

SEQ ID NO: 25 - DNA, 78 bp, artificial sequence
  synthetic oligonucleotide
gttgtagagcgtagaaatacgcaagttacaataaggcgagtccgttatcaagcggaaacgctggcaccgagtcggtgc

SEQ ID NO: 26 - DNA, 80 bp, artificial sequence
  synthetic oligonucleotide
gttgcagagacacgggagtgtcaagttgcaataaggccagtccgttatcagacgtgggaacgtcggcaccgattcggtgc

SEQ ID NO: 27 - DNA, 80 bp, artificial sequence
  synthetic oligonucleotide
gtccgagagatccgggaggatcaagttcggataaggcaagtccgttatcagctcagggatgagcggcaccgattcggtgc

SEQ ID NO: 28 - DNA, 80 bp, artificial sequence
  synthetic oligonucleotide
gtcccagaggttcgagagaaccaagttgggataaggcgagtccgttatcacgaacgagagttcgggcaccgagtcggtgc

SEQ ID NO: 29 - DNA, 80 bp, artificial sequence
  synthetic oligonucleotide
gtcgtagagttggggaaccaacaagttacgataaggccagtccgttatcatctcgggaacgagaggcaccgattcggtgc

SEQ ID NO: 30 - DNA, 82 bp, artificial sequence
  synthetic oligonucleotide
gtcgcagagcatctgaaaagatgcaagttgcgataaggcaagtccgttatcaagctcgggagagctggcaccgagtcggtgc

SEQ ID NO: 31 - DNA, 82 bp, artificial sequence
  synthetic oligonucleotide
gtgcgagagcttacgaaagtaagcaagttcgcataaggcgagtccgttatcaacagcgggagctgtggcaccgagtcggtgc

SEQ ID NO: 32 - DNA, 82 bp, artificial sequence
  synthetic oligonucleotide
gtgccagagagtaggggactactcaagttggcataaggccagtccgttatcaggtcagagatgaccggcaccgattcggtgc

SEQ ID NO: 33 - DNA, 82 bp, artificial sequence
  synthetic oligonucleotide
gtggtagaggacttgagaaagtccaagttaccataaggcaagtccgttatcacacagggaactgtgggcaccgagtcggtgc

SEQ ID NO: 34 - DNA, 82 bp, artificial sequence
  synthetic oligonucleotide
gtggcagagtcatcggaagatgacaagttcccataaggcgagtccgttatcatccgagaaatcggaggcaccgattcggtgc

SEQ ID NO: 35 - DNA, 19 bp, artificial sequence
  synthetic oligonucleotide
aatttctactcttgtagat

SEQ ID NO: 36 - DNA, 19 bp, artificial sequence
  synthetic oligonucleotide
aatttctgctcttgcagat

SEQ ID NO: 37 - DNA, 19 bp, artificial sequence
  synthetic oligonucleotide
aatttccactcttgtggat

SEQ ID NO: 38 - DNA, 20 bp, artificial sequence
  synthetic oligonucleotide
aatttctactcgttgtagat

SEQ ID NO: 39 - DNA, 20 bp, artificial sequence
  synthetic oligonucleotide
aatttctactctttgtagat

SEQ ID NO: 40 - DNA, 20 bp, artificial sequence
  synthetic oligonucleotide
taccgtaacccgttctcgtt

SEQ ID NO: 41 - DNA, 20 bp, artificial sequence
  synthetic oligonucleotide
ttcccagaattcggcagcat

SEQ ID NO: 42 - DNA, 20 bp, artificial sequence
  synthetic oligonucleotide
ccaaagacgagaggcgtatc

SEQ ID NO: 43 - DNA, 20 bp, artificial sequence
  synthetic oligonucleotide
tggttgaccaatatcggacg

SEQ ID NO: 44 - DNA, 20 bp, artificial sequence
  synthetic oligonucleotide
atcattctctgacatgccaa

SEQ ID NO: 45 - DNA, 20 bp, artificial sequence
  synthetic oligonucleotide
tcctggcttcaagtatgtta

SEQ ID NO: 46 - DNA, 80 bp, artificial sequence
  synthetic oligonucleotide
guuuuagagcuagaaauagcaaguuaaaauaaggcuaguccguuaucaacuugaaaaaguggcaccgagucggugcuuuu